(19) United States
(12) Patent Application Publication
(10) Pub. No.: US 2021/0179577 A1
(43) Pub. Date: Jun. 17, 2021

(54) AROMATIC SULFONAMIDE DERIVATIVES FOR THE TREATMENT OF ISCHEMIC STROKE

(71) Applicant: Bayer Aktiengesellschaft, Leverkusen (DE)

(72) Inventors: Peter HAUFF, Berlin (DE); Stefan WERNER, Berlin (DE)

(73) Assignee: Bayer Aktiengesellschaft, Leverkusen (DE)

(21) Appl. No.: 16/759,970

(22) PCT Filed: Oct. 24, 2018
(86) PCT No.: PCT/EP2018/079145

§ 371 Date: Apr. 28, 2020

(30) Foreign Application Priority Data
Oct. 29, 2017 (EP) 17199070.8

(51) Int. Cl. C07D 401/04; C07D 231/12; C07D 213/61; C07D 231/16; C07D 249/10; A61P 9/10

(52) U.S. Cl. CPC C07D 401/04; C07D 231/12; A61P 9/10; C07D 231/16; C07D 249/10; C07D 213/61

(57) ABSTRACT

A compound of formula (I) or an N-oxide, a salt, a hydrate, a solvate, a tautomer or a stereoisomer of said compound, or a salt of said N-oxide, tautomer or stereoisomer for use in the treatment or prophylaxis of brain ischemia, ischemic brain injury, Ischemic Stroke (IS), haemorrhagic stroke, traumatic brain injury, spinal cord injury.

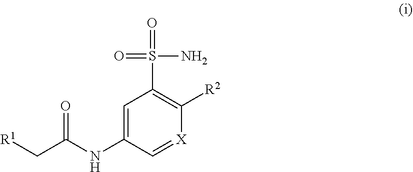

### FIELD OF APPLICATION OF THE INVENTION

The invention relates to substituted aromatic sulfonamides of formula (I) as described and defined herein, pharmaceutical compositions and combinations comprising said compounds and to the use of said compounds for manufacturing a pharmaceutical composition for the treatment or prophylaxis of brain ischemia, ischemic brain injury, Ischemic Stroke (IS), haemorrhagic stroke, traumatic brain injury, spinal cord injury. The present invention, as described and defined herein, relates to pharmaceutical compositions and combinations comprising an active ingredient which is an antagonist or a negative allosteric modulator of P2X4 for the treatment or prophylaxis of brain ischemia, ischemic brain injury, Ischemic Stroke (IS), haemorrhagic stroke, traumatic brain injury, spinal cord injury. The use of such compounds for manufacturing a pharmaceutical composition for the treatment or prophylaxis of a disease, in particular in mammals, such as but not limited to diseases associated with neuronal damage and inflammation in the brain or spinal cord, spinal cord or ischemic brain injury as such, as a sole agent or in combination with other active ingredients.

### BACKGROUND OF THE INVENTION

Adenosine triphosphate ATP is widely recognized as an important neurotransmitter implicated in various physiological and pathophysiological roles by acting through different subtypes of purinergic receptors (Burnstock 1993, Drug Dev Res 28:196-206; Burnstock 2011, Prog Neurobiol 95:229-274). To date, seven members of the P2X family have been cloned, comprising P2X1-7 (Burnstock 2013, Front Cell Neurosci 7:227). The P2X4 receptor is a ligand-gated ion channel that is expressed on a variety of cell types largely known to be involved in inflammatory/immune processes specifically including monocytes, macrophages, mast cells and microglia cells (Wang et al., 2004, BMC Immunol 5:16; Brone et al., 2007 Immunol Lett 113:83-89). Activation of P2X4 by extracellular ATP is known, amongst other things, to lead to release of pro-inflammatory cytokines and prostaglandins (PGE2) (Bo et al., 2003 Cell Tissue Res 313:159-165; Ulmann et al., 2010, EMBO Journal 29:2290-2300; de Ribero Vaccari et al., 2012, J Neurosci 32:3058-3066).

The involvement of selected P2X receptors for extracellular ATP in the onset of neuronal cell death caused by glucose/oxygen deprivation has been investigated. The in vitro studies of organotypic cultures from hippocampus evidenced that P2X2 and P2X4 were up-regulated by glucose/oxygen deprivation. Moreover, it has been shown that ischemic conditions induced specific neuronal loss not only in hippocampal, but also in cortical and striatal organotypic cultures and the P2 receptor antagonists basilen blue and suramin prevented these detrimental effects. Furthermore, hypoxia induced conditions confirmed the induction of P2X receptors in the hippocampus of gerbils in an in vivo experiment which were subjected to bilateral common carotid occlusion. In particular, P2X2 and P2X4 proteins became significantly up-regulated, although to different extent and in different cellular phenotypes. The induction was confined to the pyramidal cell layer of the CA1 subfield and to the transition zone of the CA2 subfield and it was coincident with the area of neuronal damage. P2X2 was expressed in neuronal cell bodies and fibers in the CA1 pyramidal cell layer and in the strata oriens and radiatum. Intense P2X4 immunofluorescence was localized to microglia cells. (F. Cavaliere et al., Neuroscience 120 (2003) 85-98).

In a preterm hypoxia-ischemia model in the post-natal day 3 rat, it has been characterized how the expression of purine ionotropic P2X4 receptors change in the brain post-insult. After hypoxia-ischemia, P2X4 receptor expression increased significantly and was associated with a late increase in ionised calcium binding adapter molecule-1 protein expression indicative of microglia cell activation. Minocycline, a potent inhibitor of microglia, attenuated the hypoxia-ischemia-induced increase in P2X4 receptor expression. (Julie A. Wixey et al., Journal of Neuroimmunology 212 (2009) 35-43) An overview of what is known about P2X4 expression in the CNS and evidence for pathophysiological roles in neuroinflammation and neuropathic pain is reviewed in “P2X4 Receptor Function in the Nervous System and Current Breakthroughs in Pharmacology” (L. Stokes et al., Frontiers in Pharmacology, May 2017|Volume 8|Article 291)

WO2015/088564 and WO2015/088565 provide P2X4 receptor modulating compounds, methods of their synthesis, pharmaceutical compositions comprising the compounds, and methods of their use. Said P2X4 receptor modulating compounds are useful for the treatment, prevention, and/or management of various disorders, including but not limited to, chronic pain, neuropathy, inflammatory diseases and central nervous system disorders.

There is no reference in the state of the art about substituted aromatic sulfonamides of general formula (I) as described and defined herein to be used for manufacturing a pharmaceutical composition for the treatment or prophylaxis) of brain ischemia, ischemic brain injury, Ischemic Stroke (IS), haemorrhagic stroke, traumatic brain injury, spinal cord injury, as a sole agent or in combination with other active ingredients.

Therefore, the inhibitors of P2X4 of the current invention represent valuable compounds that should complement therapeutic options either as single agents or in combination with other drugs.

### DESCRIPTION OF THE INVENTION

The present invention relates to compounds of formula (I)

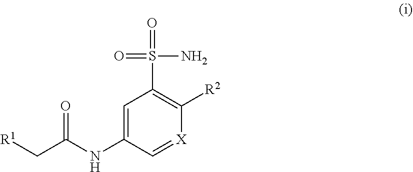

X is C or N
R¹ represents

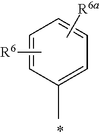

wherein * indicates the point of attachment of said group with the rest of the molecule and R⁶, R^{6a} represents independently from each other a fluorine, a chlorine, a methoxy or a hydrogen;
R² represents

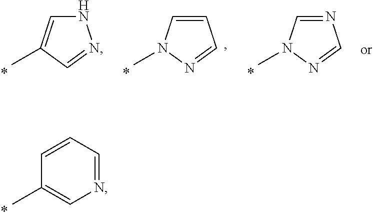

wherein * indicates the point of attachment of said group with the rest of the molecule and said group is optionally substituted one to two times with R¹¹, being, independently from each other, the same or different;
R¹¹ represents, independently from each other, halogen, cyano, C₁-C₄-alkyl, C₁-C₄-haloalkyl, C₁-C₄-hydroxyalkyl, C₁-C₄-alkoxy, C₁-C₄-haloalkoxy, (C₁-C₃-alkoxy)-ethyl-, methoxy-ethyl-, C₃-C₆-cycloalkyl;

or an N-oxide, a salt, a hydrate, a solvate, a tautomer or a stereoisomer of said compound, or a salt of said N-oxide, tautomer or stereoisomer for use in the treatment or prophylaxis of brain ischemia, ischemic brain injury, Ischemic Stroke (IS), haemorrhagic stroke, traumatic brain injury, spinal cord injury.

One aspect of the invention is the use of a compound of general formula I, or a stereoisomer, a tautomer, an N oxide, a hydrate, a solvate, or a salt thereof, particularly a pharmaceutically acceptable salt thereof, or a mixture of same for the prophylaxis or treatment of brain ischemia, ischemic brain injury, Ischemic Stroke (IS), haemorrhagic stroke, traumatic brain injury, spinal cord injury.

Another aspect of the invention refers to the use of a compound of general formula I, or a stereoisomer, a tautomer, an N oxide, a hydrate, a solvate, or a salt thereof, particularly a pharmaceutically acceptable salt thereof, or a mixture of same for the preparation of a medicament for the prophylaxis or treatment of brain ischemia, ischemic brain injury, Ischemic Stroke (IS), haemorrhagic stroke, traumatic brain injury, spinal cord injury.

Brain Ischemia may occur by a non-acquired brain injury such as part of a genetic or congenital disorder such as fetal alcohol syndrome, perinatal illness or perinatal hypoxia; these kinds of Brain Ischemia usually results in a general brain ischemia which affects usually the whole brain.

Further examples of a general brain ischemia are those that may occur by an acquired brain injury, an injury which occurs after birth, caused by different events such neonatal hypoxia; hypoxia induced e.g. due to severe lung or heart diseases; hypoxia induced due to accidents e.g. oxygen loss during diving; infectious diseases of the brain (viral, bacterial, parasitic) which can cause strong brain edema and strong immune reactions within the brain; autoimmune reactions; brain edema of different reasons such as e.g. altitude sickness, opioid drug abuse, intoxications, malignant hypertension, local blockages in interstitial fluid pathways, or by obstruction of cerebro-spinal fluid flow (e.g. obstructive hydrocephalus).

Brain Ischemia may derive also by an acquired brain injury and result in a focal brain ischemia, in which the ischemic event is localized in a specific area of the brain; Ischemic Stroke, Hemorrhagic Stroke and Traumatic Brain Injury are acquired brain injuries commonly resulting in a focal brain ischemia.

In particular, Ischemic Stroke is a focal ischemia of the brain which is associated with one or more focal brain infarctions as a result of total or partial interruption of cerebral arterial blood supply generally due to atherosclerotic lesions or embolic events, which leads to oxygen and glucose deprivation of the tissue (ischemia). Cerebral ischaemic stroke is defined according to International Classification of Diseases (ICD) as acute focal neurological dysfunction caused by focal infarction at single or multiple sites of the brain.

Evidence of acute infarction may come either from a) symptom duration lasting more than 24 hours, or b) neuroimaging or other technique in the clinically relevant area of the brain. (WHO-ICD11:https://icd.who.int/browse11/l-m/en#/http%3a%2f%2fid.who.int%2ficd%2fentity%2f636274910) Hemorrhagic stroke is due to an intracerebral or subarachnoid ruptured brain aneurysm or a weakened blood vessel leak that suddenly and leads to a focal ischemia with brain's function interferences. Blood spills into or around a defined brain area and creates swelling, pressure and ischemia, damaging cells and brain tissue.

Finally Traumatic brain injury (TBI) is a further disease which leads mostly to a focal ischemia that occurs when an external force injures the brain. TBI can be classified based on severity (mild, moderate and severe) and mechanism (closed or penetrating head injury). Mild and moderate TBIs lead mainly to brain contusions of different degrees causing edema associated brain ischemia. Moderate and severe TBIs (closed and skull penetrating) lead rather to polytraumatic injuries (e.g. vessel destruction, intracranial bleeding, brain tissue destruction) which are all close associated with ischemic conditions in the affect brain regions.

One aspect of the invention are compounds of formula (I), as described in the examples, as characterized by their names in the title and their structures as well as the subcombinations of all residues specifically disclosed in the compounds of the examples.

A further aspect of the invention refers in particular to the use of 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a salt thereof, particularly a pharmaceutically acceptable salt thereof, or a mixture of same for the preparation of a medicament for the prophylaxis or treatment of brain ischemia, ischemic brain injury, Ischemic Stroke (IS), haemorrhagic stroke, traumatic brain injury, spinal cord injury.

A further aspect of the invention are compounds of formula (I), which are present as their salts, particularly as pharmaceutical acceptable salts.

A further aspect of the present invention refers to the a parenteral formulation of a compound of general formula I, or a stereoisomer, a tautomer, an N oxide, a hydrate, a solvate, or a salt thereof, particularly a pharmaceutically acceptable salt thereof, or a mixture of same. More particularly the present invention refers to a parenteral formulation of 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a salt thereof, particularly a pharmaceutically acceptable salt thereof.

According to the present invention a parenteral formulation of a compound of general formula I, and more particularly of 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a salt thereof, particularly a pharmaceutically acceptable salt thereof, or a mixture of same is a parenteral formulation for intravenous administration.

It is to be understood that the present invention relates to any sub-combination within any embodiment or aspect of the present invention of compounds of general formula (I) supra.

Another embodiment of the invention are compounds according to the claims as disclosed in the Claims section wherein the definitions are limited according to the preferred or more preferred definitions as disclosed below or specifically disclosed residues of the exemplified compounds and subcombinations thereof.

### Definitions

Constituents which are optionally substituted as stated herein, may be substituted, unless otherwise noted, one or more times, independently from one another at any possible position. When any variable occurs more than one time in any constituent, each definition is independent. For example, when R¹, R², R⁶, R^{6a}, R¹¹, and/or X occur more than one time in any compound of formula (I) each definition of R¹, R², R⁶, R^{6a}, R¹¹, and X is independent.

Should a constituent be composed of more than one part, e.g. C₁-C₄-alkoxy-C₁-C₄-alkyl-, the position of a possible substituent can be at any of these parts at any suitable position.

A hyphen at the beginning of the constituent marks the point of attachment to the rest of the molecule. Should a ring be substituted the substituent could be at any suitable position of the ring, also on a ring nitrogen atom if suitable.

Furthermore, a constituent composed of more than one part and comprising several chemical residues, e.g. C₁-C₄-alkoxy-C₁-C₄-alkyl or phenyl-C₁-C₄-alkyl, should be read from left to right with the point of attachment to the rest of the molecule on the last part (in the example mentioned previously on the C₁-C₄-alkyl residue) The term “comprising” when used in the specification includes “consisting of”.

If it is referred to “as mentioned above” or “mentioned above” within the description it is referred to any of the disclosures made within the specification in any of the preceding pages.

The term “suitable” within the sense of the invention means chemically possible to be made by methods within the knowledge of a skilled person.

The terms as mentioned in the present text have preferably the following meanings: The term “halogen”, “halogen atom”, “halo-” or “Hal-” is to be understood as meaning a fluorine, chlorine, bromine or iodine atom, preferably a fluorine or chlorine atom.

The term “C₁-C₄-alkyl” is to be understood as preferably meaning a linear or branched, saturated, monovalent hydrocarbon group having 1, 2, 3 or 4 carbon atoms, e.g. a methyl, ethyl, propyl, butyl, iso-propyl, iso-butyl, sec-butyl, tert-butyl group, particularly 1, 2 or 3 carbon atoms (“C₁-C₃-alkyl”), e.g. a methyl, ethyl, n-propyl- or iso-propyl group.

The term “C₁-C₄-haloalkyl” is to be understood as preferably meaning a linear or branched, saturated, monovalent hydrocarbon group in which the term “C₁-C₄-alkyl” is defined supra, and in which one or more hydrogen atoms is replaced by a halogen atom, in identically or differently, i.e. one halogen atom being independent from another.

Particularly, said halogen atom is F. Said C₁-C₄-haloalkyl group is, for example, —CF₃, —CHF₂, —CH₂F, —CF₂CF₃, or —CH₂CF₃.

The term “C₁-C₄-alkoxy” is to be understood as preferably meaning a linear or branched, saturated, monovalent, hydrocarbon group of formula —O-alkyl, in which the term “alkyl” is defined supra, e.g. a methoxy, ethoxy, n-propoxy, iso-propoxy, n-butoxy, iso-butoxy, tert-butoxy or sec-butoxy group, or an isomer thereof.

The term “C₁-C₄-haloalkoxy” is to be understood as preferably meaning a linear or branched, saturated, monovalent C₁-C₄-alkoxy group, as defined supra, in which one or more of the hydrogen atoms is replaced, in identically or differently, by a halogen atom.

Particularly, said halogen atom is F. Said C₁-C₄-haloalkoxy group is, for example, —OCF₃, —OCHF₂, —OCH₂F, —OCF₂CF₃, or —OCH₂CF₃.

The term “C₁-C₄-hydroxyalkyl” is to be understood as meaning a linear or branched, saturated, monovalent hydrocarbon group in which the term “C₁-C₄-alkyl” is defined supra, and in which one or more hydrogen atoms is replaced by a hydroxy group, e.g. a hydroxymethyl, 1-hydroxyethyl, 2-hydroxyethyl, 1,2-dihydroxyethyl, 3-hydroxypropyl, 2-hydroxypropyl, 2,3-dihydroxypropyl, 1,3-dihydroxypropan-2-yl, 3-hydroxy-2-methyl-propyl, 2-hydroxy-2-methyl-propyl, 1-hydroxy-2-methyl-propyl group.

The term “C₃-C₆-cycloalkyl” is to be understood as meaning a saturated, monovalent, mono-, or bicyclic hydrocarbon ring which contains 3, 4, 5 or 6 carbon atoms (“C₃-C₆-cycloalkyl”). Said C₃-C₆-cycloalkyl group is for example, a monocyclic hydrocarbon ring, e.g. a cyclopropyl, cyclobutyl, cyclopentyl or cyclohexyl, or a bicyclic hydrocarbon ring.

The term “C₁-C₄”, as used throughout this text, e.g. in the context of the definition of “C₁-C₄-alkyl”, “C₁-C₄-haloalkyl”, “C₁-C₄-alkoxy”, or “C₁-C₄-haloalkoxy” is to be understood as meaning an alkyl group having a finite number of carbon atoms of 1 to 4, i.e. 1, 2, 3 or 4 carbon atoms. It is to be understood further that said term “C₁-C₄” is to be interpreted as any sub-range comprised therein, e.g. C₁-C₄, C₂-C₄, C₃-C₄, C₁-C₂, C₁-C₃, particularly C₁-C₂, C₁-C₃, C₁-C₄, in the case of “C₁-C₆-haloalkyl” or “C₁-C₄-haloalkoxy” even more particularly C₁-C₂.

Further, as used herein, the term “C₃-C₆”, as used throughout this text, e.g. in the context of the definition of “C₃-C₆-cycloalkyl”, is to be understood as meaning a cycloalkyl group having a finite number of carbon atoms of 3 to 6, i.e. 3, 4, 5 or 6 carbon atoms. It is to be understood further that said term “C₃-C₆” is to be interpreted as any sub-range comprised therein, e.g. C₃-C₆, C₄-C₅, C₃-C₅, C₃-C₄, C₄-C₆, C₅-C₆; particularly C₃-C₆.

The term “substituted” means that one or more hydrogens on the designated atom is replaced with a selection from the indicated group, provided that the designated atom's normal valency under the existing circumstances is not exceeded, and that the substitution results in a stable compound. Combinations of substituents and/or variables are permissible only if such combinations result in stable compounds.

The term “optionally substituted” means optional substitution with the specified groups, radicals or moieties.

Ring system substituent means a substituent attached to an aromatic or nonaromatic ring system which, for example, replaces an available hydrogen on the ring system.

As used herein, the term “one or more”, e.g. in the definition of the substituents of the compounds of the general formulae of the present invention, is understood as meaning “one, two, three, four or five, particularly one, two, three or four, more particularly one, two or three, even more particularly one or two”.

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶C, ⁸²Br, ¹²³I, ¹²⁴I, ¹²⁵I, ¹²⁹I, and ¹³¹I, respectively. Certain isotopic variations of a compound of the invention, for example, those in which one or more radioactive isotopes such as 3H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of this invention may contain one or more asymmetric centre, depending upon the location and nature of the various substituents desired. Asymmetric carbon atoms may be present in the (R) or (S) configuration, resulting in racemic mixtures in the case of a single asymmetric centre, and diastereomeric mixtures in the case of multiple asymmetric centres. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations (including enantiomers and diastereomers), are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid.

Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g.

R- or S-isomers, or E- or Z-isomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, namely:

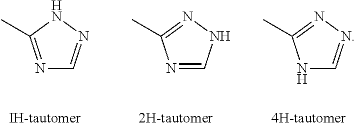

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another suitably pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol. Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and strearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae such as “hydrochloride”, “trifluoroacetate”, “sodium salt”, or “x HCl”, “x CF3COOH”, “x Na+”, for example, are to be understood as not a stoichiometric specification, but solely as a salt form.

This applies analogously to cases in which synthesis intermediates or example compounds or salts thereof have been obtained, by the preparation and/or purification processes described, as solvates, such as hydrates with (if defined) unknown stoichiometric composition.

The salts include water-insoluble and, particularly, water-soluble salts.

Furthermore, derivatives of the compounds of formula (I) and the salts thereof which are converted into a compound of formula (I) or a salt thereof in a biological system (bioprecursors or pro-drugs) are covered by the invention. Said biological system is e.g. a mammalian organism, particularly a human subject. The bioprecursor is, for example, converted into the compound of formula (I) or a salt thereof by metabolic processes.

Furthermore, the present invention includes all possible crystalline forms, or polymorphs, of the compounds of the present invention, either as single polymorphs, or as a mixture of more than one polymorphs, in any ratio.

In the context of the properties of the compounds of the present invention the term “pharmacokinetic profile” means one single parameter or a combination thereof including permeability, bioavailability, exposure, and pharmacodynamic parameters such as duration, or magnitude of pharmacological effect, as measured in a suitable experiment. Compounds with improved pharmacokinetic profiles can, for example, be used in lower doses to achieve the same effect, may achieve a longer duration of action, or a may achieve a combination of both effects.

It has now been found, and this constitutes the basis of the present invention, that said compounds of the present invention have surprising and advantageous properties.

In particular, compounds according to the present invention have surprisingly been found to effectively be active as an antagonist or a negative allosteric modulator of P2X4 in the treatment of ischemic stroke.

An allosteric modulator is a substance which indirectly influences (modulates) the effects of an agonist or inverse agonist at a target protein, for example a receptor. Allosteric modulators bind to a site distinct from that of the orthosteric agonist binding site. Usually they induce a conformational change within the protein structure. A negative modulator (NAM) reduces the effects of the orthosteric ligand, but is inactive in the absence of the orthosteric ligand.

Commercial Utility and Medical Indications
As mentioned supra, the compounds of the present invention have surprisingly been found to effectively be active as an antagonist or a negative allosteric modulator of P2X4.
A compound of general formula (I), or an N-oxide, a salt, a tautomer or a stereoisomer of said compound, or a salt of said N-oxide, tautomer or stereoisomer particularly a pharmaceutically acceptable salt thereof, or a mixture of same, as described and defined herein, is suitable for use in the treatment or prophylaxis of brain ischemia, ischemic brain injury, Ischemic Stroke (IS), haemorrhagic stroke, traumatic brain injury, or spinal cord injury.

The present invention further relates to a method for using the compounds of general formula (I) or an N-oxide, a salt, a tautomer or a stereoisomer of said compound, or a salt of said N-oxide, tautomer or stereoisomer particularly a pharmaceutically acceptable salt thereof, or a mixture of same, to treat pain- and inflammation-associated mammalian disorders and diseases.

The term “treating” or “treatment” as stated throughout this document is used conventionally, e.g., the management or care of a subject for the purpose of combating, alleviating, reducing, relieving, improving the condition of, etc., of a disease or disorder.

Preferably, the method of treating the diseases mentioned above is not limited to the treatment of said disease but also includes the treatment of pain and inflammation related to or associated with said diseases.

Pharmaceutical Compositions of the Compounds of the Invention

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease.

Therefore, the present invention includes pharmaceutical compositions that are comprised of a pharmaceutically acceptable carrier or auxiliary and a pharmaceutically effective amount of a compound, or salt thereof, of the present invention.

Another aspect of the invention is a pharmaceutical composition comprising a pharmaceutically effective amount of a compound of formula (I) and a pharmaceutically acceptable auxiliary for the treatment of a disease mentioned supra.

A pharmaceutically acceptable carrier or auxiliary is preferably a carrier that is non-toxic and innocuous to a patient at concentrations consistent with effective activity of the active ingredient so that any side effects ascribable to the carrier do not vitiate the beneficial effects of the active ingredient. Carriers and auxiliaries are all kinds of additives assisting to the composition to be suitable for administration.

A pharmaceutically effective amount of compound is preferably that amount which produces a result or exerts the intended influence on the particular condition being treated.

The compounds of the present invention can be administered with pharmaceutically-acceptable carriers or auxiliaries well known in the art using any effective conventional dosage unit forms, including immediate, slow and timed release preparations, orally, parenterally, topically, nasally, sublingually, rectally, and the like.

For oral administration, the compounds can be formulated into solid or liquid preparations such as capsules, pills, tablets, troches, lozenges, melts, powders, solutions, suspensions, or emulsions, and may be prepared according to methods known to the art for the manufacture of pharmaceutical compositions. The solid unit dosage forms can be a capsule that can be of the ordinary hard- or soft-shelled gelatine type containing auxiliaries, for example, surfactants, lubricants, and inert fillers such as lactose, sucrose, calcium phosphate, and corn starch.

In another embodiment, the compounds of this invention may be tableted with conventional tablet bases such as lactose, sucrose and cornstarch in combination with binders such as acacia, corn starch or gelatine, disintegrating agents intended to assist the break-up and dissolution of the tablet following administration such as potato starch, alginic acid, corn starch, and guar gum, gum tragacanth, acacia, lubricants intended to improve the flow of tablet granulation and to prevent the adhesion of tablet material to the surfaces of the tablet dies and punches, for example talc, stearic acid, or magnesium, calcium or zinc stearate, dyes, colouring agents, and flavouring agents such as peppermint, oil of wintergreen, or cherry flavouring, intended to enhance the aesthetic qualities of the tablets and make them more acceptable to the patient. Suitable excipients for use in oral liquid dosage forms include dicalcium phosphate and diluents such as water and alcohols, for example, ethanol, benzyl alcohol, and polyethylene alcohols, either with or without the addition of a pharmaceutically acceptable surfactant, suspending agent or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above.

Additional excipients, for example those sweetening, flavouring and colouring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived form fatty acids and hexitol anhydrides, for example, sorbitan monooleate, (4) condensation products of said partial esters with ethylene oxide, for example, polyoxyethylene sorbitan monooleate. The emulsions may also contain sweetening and flavouring agents.

Oily suspensions may be formulated by suspending the active ingredient in a vegetable oil such as, for example, arachis oil, olive oil, sesame oil or coconut oil, or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more colouring agents; one or more flavouring agents; and one or more sweetening agents such as sucrose or saccharin.

Syrups and elixirs may be formulated with sweetening agents such as, for example, glycerol, propylene glycol, sorbitol or sucrose. Such formulations may also contain a demulcent, and preservative, such as methyl and propyl parabens and flavouring and colouring agents.

The compounds of this invention may also be administered parenterally, that is, for example subcutaneously, intravenously, intraocularly, intrasynovially, intramuscularly, or interperitoneally, as injectable dosages of the compound in preferably a physiologically acceptable diluent with a pharmaceutical carrier which can be a sterile liquid or mixture of liquids such as water, saline, aqueous dextrose and related sugar solutions, an alcohol such as ethanol, isopropanol, or hexadecyl alcohol, glycols such as propylene glycol or polyethylene glycol, glycerol ketals such as 2,2-dimethyl-1,1-dioxolane-4-methanol, ethers such as poly(ethylene glycol) 400, an oil, a fatty acid, a fatty acid ester or, a fatty acid glyceride, or an acetylated fatty acid glyceride, with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include fatty acid alkali metal, ammonium, and triethanolamine salts and suitable detergents include cationic detergents, for example dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; non-ionic detergents, for example, fatty amine oxides, fatty acid alkanolamides, and poly(oxyethylene-oxypropylene)s or ethylene oxide or propylene oxide copolymers; and amphoteric detergents, for example, alkyl-beta-aminopropionates, and 2-alkylimidazoline quarternary ammonium salts, as well as mixtures.

The parenteral compositions of this invention will typically contain from about 0.5% to about 25% by weight of the active ingredient in solution. More particularly, the parenteral compositions of a compound of formula (I) according to the invention will typically contain from about 0.5% to about 20%, or from about 0.5% to about 15%, or from about 1% to about 12%, or from about 3% to about 12%, or from about 5% to about 10% by weight of the active ingredient in solution, said compound being in particular 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a salt thereof.

Preservatives and buffers may also be used advantageously. In order to minimise or eliminate irritation at the site of injection, such compositions may contain a non-ionic surfactant having a hydrophile-lipophile balance (HLB) preferably of from about 12 to about 17. The quantity of surfactant in such formulation preferably ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadeca-ethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, isotonic sodium chloride solutions and isotonic glucose solutions. In addition, sterile fixed oils are conventionally employed as solvents or suspending media. For this purpose, any bland, fixed oil may be employed including synthetic mono- or diglycerides. In addition, fatty acids such as oleic acid can be used in the preparation of injectables.

A composition of the invention may also be administered in the form of suppositories for rectal administration of the drug. These compositions can be prepared by mixing the drug with a suitable non-irritation excipient which is solid at ordinary temperatures but liquid at the rectal temperature and will therefore melt in the rectum to release the drug. Such materials are, for example, cocoa butter and polyethylene glycol.

Controlled release formulations for parenteral administration include liposomal, polymeric microsphere and polymeric gel formulations that are known in the art.

It may be desirable or necessary to introduce the pharmaceutical composition to the patient via a mechanical delivery device. The construction and use of mechanical delivery devices for the delivery of pharmaceutical agents is well known in the art. Direct techniques for administration, for example, administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, issued Apr. 30, 1991.

The compositions of the invention can also contain other conventional pharmaceutically acceptable compounding ingredients, generally referred to as carriers or diluents, as necessary or desired. Conventional procedures for preparing such compositions in appropriate dosage forms can be utilized.

Such ingredients and procedures include those described in the following references, each of which is incorporated herein by reference: Powell, M. F. et al., “Compendium of Excipients for Parenteral Formulations” PDA Journal of Pharmaceutical Science & Technology 1998, 52(5), 238-311; Strickley, R. G “Parenteral Formulations of Small Molecule Therapeutics Marketed in the United States (1999)-Part-1” PDA Journal of Pharmaceutical Science & Technology 1999, 53(6), 324-349; and Nema, S. et al., “Excipients and Their Use in Injectable Products” PDA Journal of Pharmaceutical Science & Technology 1997, 51(4), 166-171.

Commonly used pharmaceutical ingredients that can be used as appropriate to formulate the composition for its intended route of administration include:

acidifying agents (examples include but are not limited to acetic acid, citric acid, fumaric acid, hydrochloric acid, nitric acid);

alkalinizing agents (examples include but are not limited to ammonia solution, ammonium carbonate, diethanolamine, monoethanolamine, potassium hydroxide, sodium borate, sodium carbonate, sodium hydroxide, triethanolamine, trolamine);

adsorbents (examples include but are not limited to powdered cellulose and activated charcoal);

aerosol propellants (examples include but are not limited to carbon dioxide, CCl₂F₂, F₂ClC—CClF₂ and CClF₃)

air displacement agents—examples include but are not limited to nitrogen and argon;

antifungal preservatives (examples include but are not limited to benzoic acid, butylparaben, ethylparaben, methylparaben, propylparaben, sodium benzoate);

antimicrobial preservatives (examples include but are not limited to benzalkonium chloride, benzethonium chloride, benzyl alcohol, cetylpyridinium chloride, chlorobutanol, phenol, phenylethyl alcohol, phenylmercuric nitrate and thimerosal);

antioxidants (examples include but are not limited to ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite);

binding materials (examples include but are not limited to block polymers, natural and synthetic rubber, polyacrylates, polyurethanes, silicones, polysiloxanes and styrene-butadiene copolymers);

buffering agents (examples include but are not limited to potassium metaphosphate, dipotassium phosphate, sodium acetate, sodium citrate anhydrous and sodium citrate dihydrate);

carrying agents (examples include but are not limited to acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection);

chelating agents (examples include but are not limited to edetate disodium and edetic acid);

colourants (examples include but are not limited to FD&C Red No. 3, FD&C Red No. 20, FD&C Yellow No. 6, FD&C Blue No. 2, D&C Green No. 5, D&C Orange No. 5, D&C Red No. 8, caramel and ferric oxide red);

clarifying agents (examples include but are not limited to bentonite);

emulsifying agents (examples include but are not limited to acacia, cetomacrogol, cetyl alcohol, glyceryl monostearate, lecithin, sorbitan monooleate, polyoxyethylene 50 monostearate);

encapsulating agents (examples include but are not limited to gelatin and cellulose acetate phthalate),

flavourants (examples include but are not limited to anise oil, cinnamon oil, cocoa, menthol, orange oil, peppermint oil and vanillin);

humectants (examples include but are not limited to glycerol, propylene glycol and sorbitol);

levigating agents (examples include but are not limited to mineral oil and glycerin);

oils (examples include but are not limited to arachis oil, mineral oil, olive oil, peanut oil, sesame oil and vegetable oil);

ointment bases (examples include but are not limited to lanolin, hydrophilic ointment, polyethylene glycol ointment, petrolatum, hydrophilic petrolatum, white ointment, yellow ointment, and rose water ointment);

penetration enhancers (transdermal delivery) (examples include but are not limited to monohydroxy or polyhydroxy alcohols, mono- or polyvalent alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas),

plasticizers (examples include but are not limited to diethyl phthalate and glycerol);

solvents (examples include but are not limited to ethanol, corn oil, cottonseed oil, glycerol, isopropanol, mineral oil, oleic acid, peanut oil, purified water, water for injection, sterile water for injection and sterile water for irrigation);

stiffening agents (examples include but are not limited to cetyl alcohol, cetyl esters wax, microcrystalline wax, paraffin, stearyl alcohol, white wax and yellow wax);

suppository bases (examples include but are not limited to cocoa butter and polyethylene glycols (mixtures));

surfactants (examples include but are not limited to benzalkonium chloride, nonoxynol 10, oxtoxynol 9, polysorbate 80, sodium lauryl sulfate and sorbitan mono-palmitate);

suspending agents (examples include but are not limited to agar, bentonite, carbomers, carboxymethylcellulose sodium, hydroxyethyl cellulose, hydroxypropyl cellulose, hydroxypropyl methylcellulose, kaolin, methylcellulose, tragacanth and veegum);

sweetening agents (examples include but are not limited to aspartame, dextrose, glycerol, mannitol, propylene glycol, saccharin sodium, sorbitol and sucrose);

tablet anti-adherents (examples include but are not limited to magnesium stearate and talc);

tablet binders (examples include but are not limited to acacia, alginic acid, carboxymethylcellulose sodium, compressible sugar, ethylcellulose, gelatin, liquid glucose, methylcellulose, non-crosslinked polyvinyl pyrrolidone, and pregelatinized starch);

tablet and capsule diluents (examples include but are not limited to dibasic calcium phosphate, kaolin, lactose, mannitol, microcrystalline cellulose, powdered cellulose, precipitated calcium carbonate, sodium carbonate, sodium phosphate, sorbitol and starch);

tablet coating agents (examples include but are not limited to liquid glucose, hydroxyethyl cellulose, hydroxypropyl cellulose, hydroxypropyl methylcellulose, methylcellulose, ethylcellulose, cellulose acetate phthalate and shellac);

tablet direct compression excipients (examples include but are not limited to dibasic calcium phosphate);

tablet disintegrants (examples include but are not limited to alginic acid, carboxymethylcellulose calcium, microcrystalline cellulose, polacrillin potassium, crosslinked polyvinylpyrrolidone, sodium alginate, sodium starch glycollate and starch);

tablet qlidants (examples include but are not limited to colloidal silica, corn starch and talc);

tablet lubricants (examples include but are not limited to calcium stearate, magnesium stearate, mineral oil, stearic acid and zinc stearate);

tablet/capsule opaquants (examples include but are not limited to titanium dioxide);

tablet polishing agents (examples include but are not limited to carnuba wax and white wax);

thickening agents (examples include but are not limited to beeswax, cetyl alcohol and paraffin);

tonicity agents (examples include but are not limited to dextrose and sodium chloride);

viscosity increasing agents (examples include but are not limited to alginic acid, bentonite, carbomers, carboxymethylcellulose sodium, methylcellulose, polyvinyl pyrrolidone, sodium alginate and tragacanth); and

wetting agents (examples include but are not limited to heptadecaethylene oxycetanol, lecithins, sorbitol monooleate, polyoxyethylene sorbitol monooleate, and polyoxyethylene stearate).

Pharmaceutical compositions according to the present invention can be illustrated as follows:

Sterile i.v. solution: A 5 mg/ml solution of the desired compound of this invention can be made using sterile, injectable water, and the pH is adjusted if necessary. The solution is diluted for administration to 1-2 mg/ml with sterile 5% dextrose and is administered as an i.v. infusion over about 60 minutes.

Lyophilised powder for i.v. administration: A sterile preparation can be prepared with (i) 100-1000 mg of the desired compound of this invention as a lyophilised powder, (ii) 32-327 mg/ml sodium citrate, and (iii) 300-3000 mg Dextran 40. The formulation is reconstituted with sterile, injectable saline or dextrose 5% to a concentration of 10 to 20 mg/ml, which is further diluted with saline or dextrose 5% to 0.2-0.4 mg/ml, and is administered either IV bolus or by IV infusion over 15-60 minutes.

Intramuscular suspension: The following solution or suspension can be prepared, for intramuscular injection:

50 mg/ml of the desired, water-insoluble compound of this invention

5 mg/ml sodium carboxymethylcellulose

4 mg/ml TWEEN 80

9 mg/ml sodium chloride

9 mg/ml benzyl alcohol

Hard Shell Capsules: A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules: A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Tablets: A large number of tablets are prepared by conventional procedures so that the dosage unit is 100 mg of active ingredient, 0.2 mg. of colloidal silicon dioxide, 5 mg of magnesium stearate, 275 mg of microcrystalline cellulose, 11 mg. of starch, and 98.8 mg of lactose. Appropriate aqueous and non-aqueous coatings may be applied to increase palatability, improve elegance and stability or delay absorption.

Immediate Release Tablets/Capsules: These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

Dose and Administration

Based upon standard laboratory techniques known to evaluate compounds useful for the treatment of disorders and/or disease, which are influenced by P2X4, by standard toxicity tests and by standard pharmacological assays for the determination of treatment of the conditions identified above in mammals, and by comparison of these results with the results of known medicaments that are used to treat these conditions. The effective dosage of the compounds of this invention can readily be determined for treatment of each desired indication. The amount of the active ingredient to be administered in the treatment of one of these conditions can vary widely according to such considerations as the particular compound and dosage unit employed the mode of administration, the period of treatment, the age and sex of the patient treated, and the nature and extent of the condition treated.

The total amount of a compound of formula I to be administered will generally range from about 0.1 mg/kg to about 50 mg/kg body weight per day, more particularly from about 0.2 mg/kg to about 30 mg/kg body weight per day, more particularly from about 0.5 mg/kg to about 15 mg/kg body weight per day.

Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 5 mg to about 500 mg of active ingredient, particularly about 25 mg to about 150 mg, and can be administered one or more times per day or less than once a day.

According to a particular form of embodiment of the invention, a oral unit dosage for a administration of the compounds of the present invention includes but is not limited to 0.5 mg/kg to about 10 mg/kg body weight one to three times a day to once a week.

The average daily dosage for administration by injection, including intravenous, intramuscular, subcutaneous and parenteral injections, and use of infusion techniques will be according to a particular form of embodiment of the invention from 0.5 to 50 mg/kg of total body weight.

The average daily rectal dosage regimen will preferably be from 0.5 to 50 mg/kg of total body weight.

The average daily topical dosage regimen will preferably be from 0.5 to 50 mg/kg administered between one to four times daily.

The average daily inhalation dosage regimen will preferably be from 0.5 to 30 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

The blood-brain barrier (BBB) is formed by the brain capillary endothelium and works as filter that excludes from the brain ˜100% of large-molecule and more than 98% of all small-molecule intended as neurotherapeutics. During the acute phase of brain ischemia, ischemic brain injury, Ischemic Stroke (IS), haemorrhagic stroke, traumatic brain injury, and spinal cord injury, the BBB is compromised and its junctions executing the excluding function are weakened. In the time windows from the onset of the above identified disorders, from the onset of IS, to the closure of the BBB, the delivering of therapeutic agents to specific regions of the brain is particularly favourable.

A compound of general formula (I), or an N-oxide, a salt, a tautomer or a stereoisomer of said compound, or a salt of said N-oxide, tautomer or stereoisomer particularly a pharmaceutically acceptable salt thereof, or a mixture of same, as described and defined herein is advantageously administered from the onset of the ischemic brain injury, Ischemic Stroke (IS), haemorrhagic stroke, traumatic brain injury, and spinal cord injury, in particular of IS, from the onset of the disease up to the reestablishment of the BBB so that the compound crosses the BBB in adequate amounts.

More particularly a compound of general formula (I), such as 2-(2-Chlorophenyl)-N-[4-(4-cyano-1H-pyrazol-1-yl)-3-sulfamoylphenyl]acetamide, is advantageously administered from the onset of the disease, like for example IS, up to about one month, more particularly up to about three weeks, more particular up to about two weeks, more particularly up to about ten days.

More particularly a compound of general formula (I), such as 2-(2-Chlorophenyl)-N-[4-(4-cyano-1H-pyrazol-1-yl)-3-sulfamoylphenyl]acetamide, is advantageously administered within 6 hours, more particularly within 3 hours from the onset of the disease.

As onset of the disease can be considered not only the exact time in which the ischemic brain injury, Ischemic Stroke (IS), haemorrhagic stroke, traumatic brain injury, or spinal cord injury takes place but also the time in which the symptoms of a such disease have been identified or such disease has been confirmed for example by means of Computer Tomography (CT) or Magnetic Resonance Imaging (MRI).

Combination Therapies

The term “combination” in the present invention is used as known to persons skilled in the art and may be present as a fixed combination, a non-fixed combination or kit of parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit of parts” in the present invention is used as known to persons skilled in the art and is defined as a combination wherein the said first active ingredient and the said second active ingredient are present in more than one unit. One example of a non-fixed combination or kit of parts is a combination wherein the said first active ingredient and the said second active ingredient are present separately. The components of the non-fixed combination or kit of parts may be administered separately, sequentially, simultaneously, concurrently or chronologically staggered.

The compounds of the present invention can be administered as the sole pharmaceutical agent or in combination with one or more other pharmaceutical agents where the combination causes no unacceptable adverse effects. The present invention relates also to such combinations.

Furthermore, the compounds of the present invention can be combined with therapeutic agents or active ingredients, that are already approved or that are still under development for the treatment and/or prophylaxis of diseases which are related to or mediated by P2X4.

For the treatment and/or prophylaxis of brain ischemia, ischemic brain injury, Ischemic Stroke (IS), haemorrhagic stroke, traumatic brain injury, spinal cord injury, the compounds of the present invention can be administered in combination or as co-medication in addition to the respective standard of cares (SOC)=basic intensive care unit therapy including blood pressure stabilization (usually reduction of BP); recanalization (pharmacological intravenous lysis with e.g. rtPA and/or mechanical recanalization by intra-arterial thrombus extraction); brain edema treatment by osmotherapy with e.g. glycerol, mannitol or hypertonic salt solution and/or treatment with glucokortikoids.

For the treatment and/or prophylaxis of brain ischemia, ischemic brain injury, Ischemic Stroke (IS), haemorrhagic stroke, traumatic brain injury, spinal cord injury, the compounds of the present invention can be administered in combination or as co-medication with any substance that can be applied as antithrombotic agents, in particular anticoagulants like glycosaminoglycans for example Heparin, Low-molecular-weight heparins or Danaparoid; direct thrombin inhibitors like for example Argatroban, Antithrombin or Protein C; Antiplatelet agents Aspirin, or Clopidogrel; Glycoprotein IIb/IIa receptor blockers like Abciximab or Eptifibatide (Integrilin), fibrinolytic drugs such as Streptokinase, Anistreplase or Alteplase. A very particular example is the administration or comedicaton of the compound of the invention together with Aspirin.

The compounds of the present invention can be combined with other pharmacological agents and compounds that are intended to treat inflammatory diseases, inflammatory pain or general pain conditions.

Methods of testing for a particular pharmacological or pharmaceutical property are well known to persons skilled in the art.

The example testing experiments described herein serve to illustrate the present invention and the invention is not limited to the examples given.

As will be appreciated by persons skilled in the art, the invention is not limited to the particular embodiments described herein, but covers all modifications of said embodiments that are within the spirit and scope of the invention as defined by the appended claims.

The following examples illustrate the invention in greater detail, without restricting it. Further compounds according to the invention, of which the preparation is not explicitly described, can be prepared in an analogous way.

The compounds, which are mentioned in the examples and the salts thereof represent preferred embodiments of the invention as well as a claim covering all subcombinations of the residues of the compound of formula (I) as disclosed by the specific examples.

The term “according to” within the experimental section is used in the sense that the procedure referred to is to be used “analogously to”.

Synthesis of Compounds

The following schemes and general procedures illustrate general synthetic routes to the compounds of general formula (I) of the invention and are not intended to be limiting. It is obvious to the person skilled in the art that the order of transformations as exemplified in schemes 1 to 5 can be modified in various ways. The order of transformations exemplified in schemes 1 to 5 is therefore not intended to be limiting. In addition, interconversion of substituents, for example of residues R¹, R² or R¹ can be achieved before and/or after the exemplified transformations. These modifications can be such as the introduction of protecting groups, cleavage of protecting groups, reduction or oxidation of functional groups, halogenation, metallation, substitution or other reactions known to the person skilled in the art. These transformations include those which introduce a functionality which allows for further interconversion of substituents. Appropriate protecting groups and their introduction and cleavage are well-known to the person skilled in the art (see for example T. W. Greene and P. G. M. Wuts, Protective Groups in Organic Synthesis, 3rd edition, Wiley 1999).

All reagents used for the preparation of the compounds of the invention are commercially available, known in the literature or can be prepared as described.

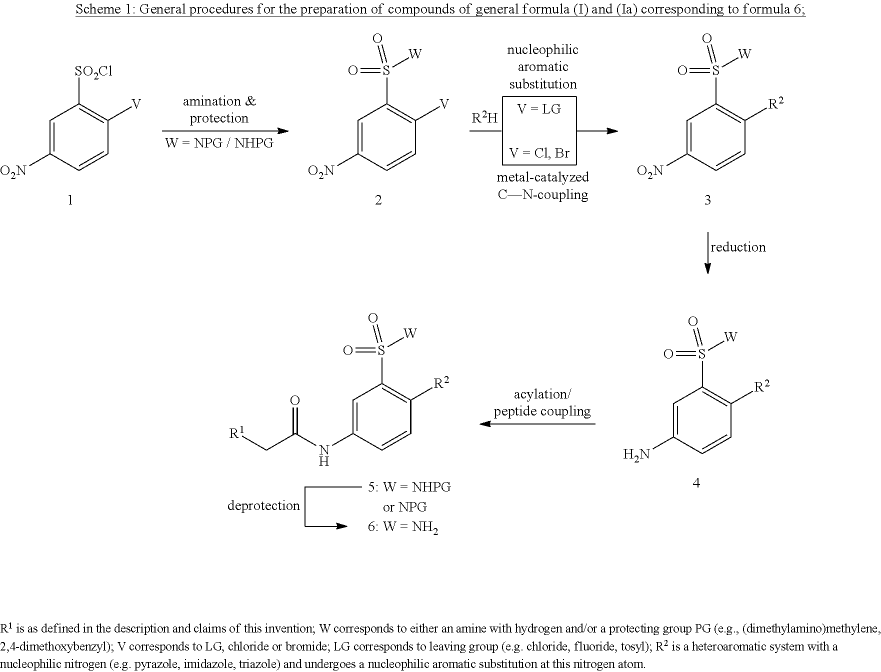

Compounds of general formula 6 can by synthesized as depicted in Scheme 1. The person skilled in the art will be able to convert sulfonyl chlorides 1 to the protected sulfonyl amides 2 and will be able to select a protecting group PG that is suitable for the following steps. Examples for suitable protecting groups PG are 2,4-dimethoxybenzyl or (dimethylamino)methylene. In case V corresponds to a leaving group LG (e.g. fluoride, chloride, tosyl) compounds 2 can be converted in a nucleophilic aromatic substitution reaction in a suitable solvent (e.g. acetonitrile) and in presence of a suitable base (e.g. potassium carbonate, cesium carbonate, . . . ) with a heteroaromatic system R²H that contains a nucleophilic nitrogen (e.g. pyrazole, imidazole, triazole, . . . ) to compounds 3 while forming a new C—N-bond. In case V corresponds to chloride or bromide, compounds 3 can be formed in a metal-catalyzed C—N coupling reaction with a nitrogen-containing heteroaromatic system (e.g. 1,2,3-triazoles) and in the presence of a suitable catalytic system (e.g. tris(dibenzylideneacetone)dipalladium/di-tert-butyl(2′,4′,6′-triisopropyl-3,4,5,6-tetramethyl-[1,1′-biphenyl]-2-yl)phosphine/potassium phoasphate/toluene). In the next step, nitro compounds 3 can be converted to the corresponding anilines 4 by reduction under hydrogenation conditions, in polar solvents such as ethanol, methanol, dioxane or tetrahydrofuran in the presence of for example Pd-, Pt-, Fe- or Sn-based catalysts. Anilines 4 can be converted to the corresponding amides 5 for example by reaction with acyl chlorides or by standard peptide bond formation using all known procedures, such as reaction of the corresponding carboxylic acid in the presence of a coupling reagent e.g. HATU. In the last step, amides 5 are deprotected to the desired sulfonamides 6. Deprotection conditions depend on the used protecting group (e.g. TFA/dichloromethane in case of 2,4-dimethoxybenzyl or aqueous ammonia/methanol in case of (dimethylamino)methylene).

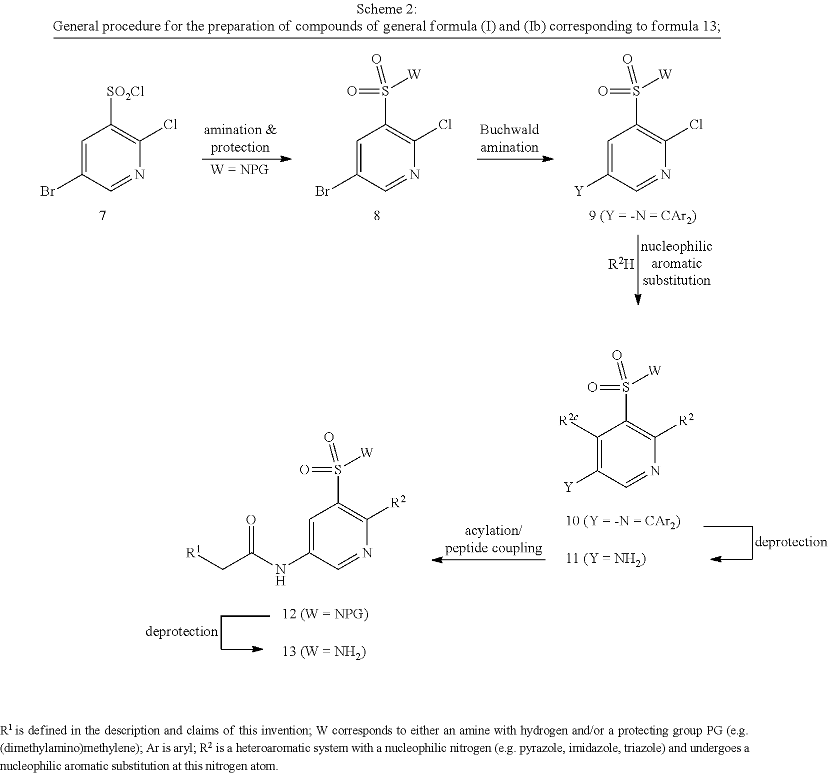

Compounds of general formula 13 can by synthesized as depicted in Scheme 2. The person skilled in the art will be able to convert sulfonyl chlorides 7 to the protected sulfonyl amides 8 and will be able to select a protecting group PG that is suitable for the following steps. Example for a suitable protecting group PG is (dimethylamino)methylene (reaction of sulfonylchlorides 7 with ammonia, then reaction with 1,1-dimethoxy-N,N-dimethylmethanamine in DMF). Using protection and deprotection strategies, Buchwald amination of 8 in the presence of suitable catalysts (see for example WO2011120026A1) leads to intermediates 9. Nucleophilic aromatic substitution reaction in a suitable solvent (e.g. acetonitrile) and in presence of a suitable base (e.g. potassium carbonate, . . . ) with a heteroaromatic system R²H that contains a nucleophilic nitrogen (e.g. pyrazole, imidazole, triazole, . . . ) leads to pyridines 10. Deprotection of 10 (under acidic conditions in case Y=—N═CAr₂) is followed by conversion of the resulting anilines 11 to amides 12 for example by reaction with acyl chlorides or by standard peptide bond formation using all known procedures, such as reaction of the corresponding carboxylic acids in the presence of a coupling reagent e.g. HATU. In the last step, amide 12 is deprotected to the desired sulfonamides 13. Deprotection conditions depend on the used protecting group (e.g. aqueous ammonia/methanol in case of (dimethylamino)methylene).

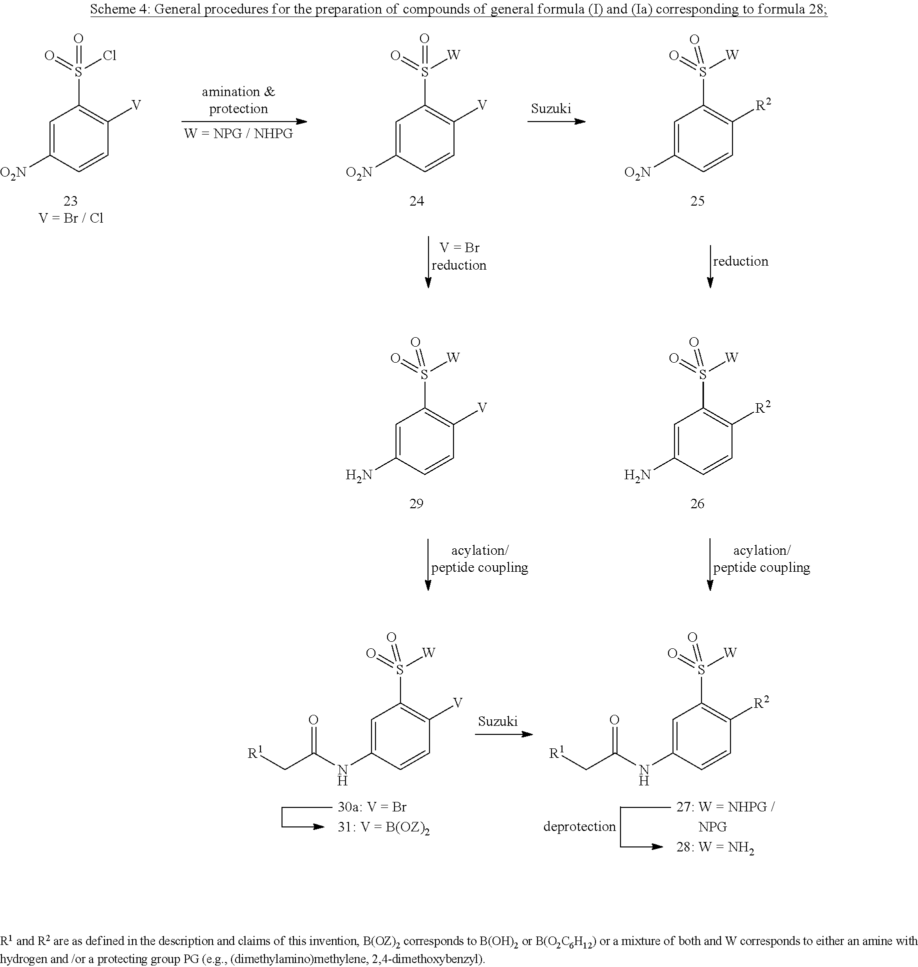

Compounds of general formula 28 can by synthesized as depicted in Scheme 4. Starting from corresponding sulfonyl chlorides 23 (with V being either bromide or chloride) C-connected aryl and heteroaryl derivatives can be prepared via e.g. Suzuki cross-coupling reactions known to the person skilled in the art. Transformation of the protected sulfonamides 24 into aryl/heteroaryl compounds with general formula 25 can be achieved by reaction with the corresponding boronic acid (or ester or a mixture of both) under palladium catalysis in protic (e.g. isopropanol) or aprotic solvents. The corresponding amines 26 can be obtained from intermediates 25 by reduction under hydrogenation conditions, in polar solvents such as ethanol or tetrahydrofuran in the presence of for example Pd-, Pt-, Fe- or Sn-based catalysts. Subsequent acylation to the corresponding amides 27 can be achieved for example by reaction with acyl chlorides or by standard peptide bond formation using all known procedures, such as reaction of the corresponding carboxylic acid in the presence of a coupling reagent e.g. HATU. For W equals a protecting group subsequent deprotection with e.g. trifluoroacetic acid (TFA), results in compounds of general formula 28.

Alternatively, starting from intermediates 24 with V═Br, reduction under hydrogenation conditions, in polar solvents such as ethanol or tetrahydrofuran in the presence of for example Pt-, Fe- or Sn-based catalysts yields amines 29. The corresponding amides 30a can be obtained by reaction with acyl chlorides or by standard peptide bond formation using all known procedures. Subsequent arylation/heteroarylation using e.g. palladium catalyzed cross-couplings gives access to intermediates 27. Alternatively bromides 30a can be converted into the corresponding boronic acid/ester intermediates 31 (B(OZ)₂═B(OH)₂ or B(O₂C₆H₁₂)) and further reacted using e.g. palladium catalysis known to the person skilled in the art to obtain intermediates 27 which after deprotection yield final products with general formula 28.

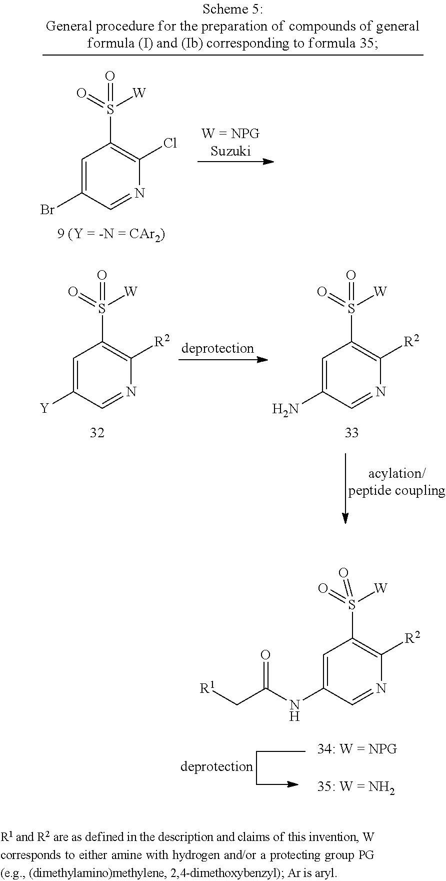

Compounds of general formula 35 can by synthesized as depicted in Scheme 5. Starting from intermediate 9 C-coupled aryl and heteroaryl derivatives 32 can be prepared via e.g. palladium cross-couplings, e.g. Suzuki reactions, known to the person skilled in the art (see for example US 20110281865). Deprotection under e.g. acidic condition yields amines 33. Subsequent acylation to the corresponding amides can be achieved for example by reaction with acyl chlorides or by standard peptide bond formation using all known procedures, such as reaction of the corresponding carboxylic acid in the presence of a coupling reagent e.g. HATU. For W equals a protected amino function subsequent deprotection (with e.g. aqueous ammonia in case of (dimethylamino)methylene as protection group), results in compounds of general formula 35.

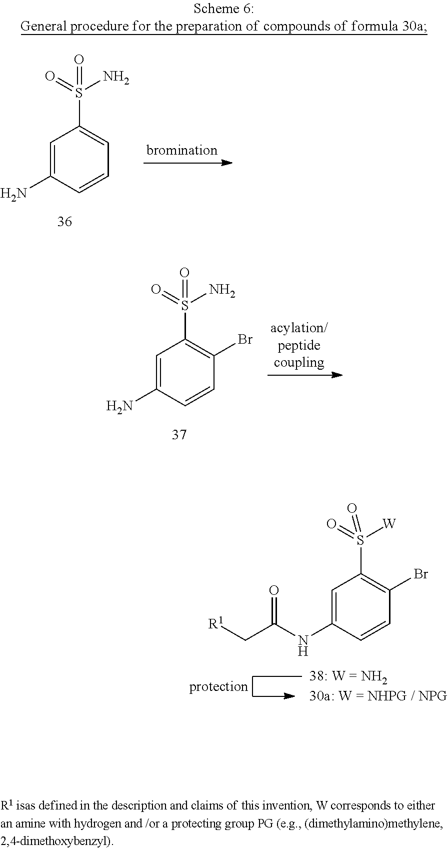

Compounds of general formula 30a can by synthesized as depicted in Scheme 6. Starting from the corresponding aniline 36, bromination (e.g. with NBS in DMF) leads to bromoaniline 37. Subsequent acylation to the corresponding amides 38 can be achieved for example by reaction with acyl chlorides or by standard peptide bond formation using all known procedures, such as reaction of the corresponding carboxylic acid in the presence of a coupling reagent e.g. HATU. Subsequent protection of the sulfonamide moiety (e.g. with 1,1-dimethoxy-N,N-dimethylmethanamine in DMF) leads to protected amides 30a that then can be further transformed e.g. using Suzuki chemistry as described in Scheme 4.

The compounds according to the invention are isolated and purified in a manner known per se, e.g. by distilling off the solvent in vacuo and recrystallizing the residue obtained from a suitable solvent or subjecting it to one of the customary purification methods, such as chromatography on a suitable support material. Furthermore, reverse phase preparative HPLC of compounds of the present invention which possess a sufficiently basic or acidic functionality, may result in the formation of a salt, such as, in the case of a compound of the present invention which is sufficiently basic, a trifluoroacetate or formate salt for example, or, in the case of a compound of the present invention which is sufficiently acidic, an ammonium salt for example. Salts of this type can either be transformed into its free base or free acid form, respectively, by various methods known to the person skilled in the art, or be used as salts in subsequent biological assays. Additionally, the drying process during the isolation of compounds of the present invention may not fully remove traces of cosolvents, especially such as formic acid or trifluoroacetic acid, to give solvates or inclusion complexes. The person skilled in the art will recognise which solvates or inclusion complexes are acceptable to be used in subsequent biological assays. It is to be understood that the specific form (e.g. salt, free base, solvate, inclusion complex) of a compound of the present invention as isolated as described herein is not necessarily the only form in which said compound can be applied to a biological assay in order to quantify the specific biological activity.

Salts of the compounds of formula (I), (Ia) and (Ib) according to the invention can be obtained by dissolving the free compound in a suitable solvent (for example a ketone such as acetone, methylethylketone or methylisobutylketone, an ether such as diethyl ether, tetrahydrofuran or dioxane, a chlorinated hydrocarbon such as methylene chloride or chloroform, or a low molecular weight aliphatic alcohol such as methanol, ethanol or isopropanol) which contains the desired acid or base, or to which the desired acid or base is then added. The acid or base can be employed in salt preparation, depending on whether a mono- or polybasic acid or base is concerned and depending on which salt is desired, in an equimolar quantitative ratio or one differing therefrom. The salts are obtained by filtering, reprecipitating, precipitating with a non-solvent for the salt or by evaporating the solvent. Salts obtained can be converted into the free compounds which, in turn, can be converted into salts. In this manner, pharmaceutically unacceptable salts, which can be obtained, for example, as process products in the manufacturing on an industrial scale, can be converted into pharmaceutically acceptable salts by processes known to the person skilled in the art. Especially preferred are hydrochlorides and the process used in the example section.

Pure diastereomers and pure enantiomers of the compounds and salts according to the invention can be obtained e.g. by asymmetric synthesis, by using chiral starting compounds in synthesis and by splitting up enantiomeric and diasteriomeric mixtures obtained in synthesis.

Enantiomeric and diastereomeric mixtures can be split up into the pure enantiomers and pure diastereomers by methods known to a person skilled in the art. Preferably, diastereomeric mixtures are separated by crystallization, in particular fractional crystallization, or chromatography. Enantiomeric mixtures can be separated e.g. by forming diastereomers with a chiral auxilliary agent, resolving the diastereomers obtained and removing the chiral auxilliary agent. As chiral auxilliary agents, for example, chiral acids can be used to separate enantiomeric bases such as e.g. mandelic acid and chiral bases can be used to separate enantiomeric acids by formation of diastereomeric salts.

Furthermore, diastereomeric derivatives such as diastereomeric esters can be formed from enantiomeric mixtures of alcohols or enantiomeric mixtures of acids, respectively, using chiral acids or chiral alcohols, respectively, as chiral auxilliary agents. Additionally, diastereomeric complexes or diastereomeric clathrates may be used for separating enantiomeric mixtures. Alternatively, enantiomeric mixtures can be split up using chiral separating columns in chromatography. Another suitable method for the isolation of enantiomers is the enzymatic separation.

One preferred aspect of the invention is the process for the preparation of the compounds of general formula (I) (Ia) or (Ib) or an N-oxide, a salt, a tautomer or a stereoisomer of said compound, or a salt of said N-oxide, tautomer or stereoisomer according to the examples, as well as the intermediates used for their preparation.

Optionally, compounds of the formula (I), (Ia) and (Ib) can be converted into their salts, or, optionally, salts of the compounds of the formula (I), (Ia) and (Ib) can be converted into the free compounds. Corresponding processes are customary for the skilled person.

### EXPERIMENTAL PART

### Abbreviations

The following table lists the abbreviations used in this paragraph and in the Intermediate Examples and Examples section as far as they are not explained within the text body.

| Abbreviation | Meaning |
|---|---|
| AcOH | acetic acid (ethanoic acid) |
| aq. | aqueous |
| boc | t-butoxycarbonyl |
| br | broad |
| Cl | chemical ionisation |
| d | doublet |
| DAD | diode array detector |
| DBU | 1,8-Diazabicyclo(5.4.0)undec-7-ene |
| DCM | dichloromethane |
| dd | double-doublet |
| DIPEA | diisopropylethylamine |
| DMF | N,N-dimethylformamide |
| DMSO | dimethyl sulfoxide |
| ELSD | Evaporative Light Scattering Detector |
| EtOAc | ethyl acetate |
| EtOH | ethanol |
| eq. | equivalent |
| ESI | electrospray (ES) ionisation |
| HATU | 1-[Bis(dimethylamino)methylene]-1H-1,2,3-triazolo[4,5- |
| | b]pyridinium 3-oxid hexafluorophosphate |
| HPLC | high performance liquid chromatography |
| LC-MS | liquid chromatography mass spectrometry |
| m | multiplet |
| MeCN | acetonitrile |
| MeOH | methanol |
| MS | mass spectrometry |
| MTBE | methyl tert-butylether |
| NMR | nuclear magnetic resonance spectroscopy: chemical |
| | shifts (δ) are given in ppm. The chemical shifts were |
| | corrected by setting the DMSO signal to 2.50 ppm |
| | unless otherwise stated. |
| PDA | Photo Diode Array |
| PoraPak ™; | a HPLC column obtainable from Waters |
| q | quartet |
| r.t. or rt | room temperature |
| Rt | retention time (as measured either with HPLC or UPLC) |
| | in minutes |
| s | singlet |
| SM | starting material |
| SQD | Single-Quadrupol-Detector |
| t | triplet |
| td | dublett of a triplet |
| dt | triplett of a dublet |
| TEA | triethylamine |
| THF | tetrahydrofuran |
| UPLC | ultra performance liquid chromatography |

Other abbreviations have their meanings customary per se to the skilled person.

The various aspects of the invention described in this application are illustrated by the following examples which are not meant to limit the invention in any way.

Specific Experimental Descriptions

NMR peak forms in the following specific experimental descriptions are stated as they appear in the spectra, possible higher order effects have not been considered. Reactions employing microwave irradiation may be run with a Biotage Initator® microwave oven optionally equipped with a robotic unit. The reported reaction times employing microwave heating are intended to be understood as fixed reaction times after reaching the indicated reaction temperature. The compounds and intermediates produced according to the methods of the invention may require purification. Purification of organic compounds is well known to the person skilled in the art and there may be several ways of purifying the same compound. In some cases, no purification may be necessary. In some cases, the compounds may be purified by crystallization. In some cases, impurities may be stirred out using a suitable solvent. In some cases, the compounds may be purified by chromatography, particularly flash column chromatography, using for example prepacked silica gel cartridges, e.g. from Separtis such as Isolute® Flash silica gel or Isolute® Flash NH₂ silica gel in combination with a Isolera® autopurifier (Biotage) and eluents such as gradients of e.g. hexane/ethyl acetate or DCM/methanol. In some cases, the compounds may be purified by preparative HPLC using for example a Waters autopurifier equipped with a diode array detector and/or on-line electrospray ionization mass spectrometer in combination with a suitable prepacked reverse phase column and eluents such as gradients of water and acetonitrile which may contain additives such as trifluoroacetic acid, formic acid or aqueous ammonia. In some cases, purification methods as described above can provide those compounds of the present invention which possess a sufficiently basic or acidic functionality in the form of a salt, such as, in the case of a compound of the present invention which is sufficiently basic, a trifluoroacetate or formate salt for example, or, in the case of a compound of the present invention which is sufficiently acidic, an ammonium salt for example. A salt of this type can either be transformed into its free base or free acid form, respectively, by various methods known to the person skilled in the art, or be used as salts in subsequent biological assays. It is to be understood that the specific form (e.g. salt, free base etc) of a compound of the present invention as isolated as described herein is not necessarily the only form in which said compound can be applied to a biological assay in order to quantify the specific biological activity.

The percentage yields reported in the following examples are based on the starting component that was used in the lowest molar amount. Most reaction conditions were not optimized for yield. Air and moisture sensitive liquids and solutions were transferred via syringe or cannula, and introduced into reaction vessels through rubber septa.

Commercial grade reagents and solvents were used without further purification. The term “concentrated in vacuo” refers to use of a Buchi rotary evaporator at a minimum pressure of approximately 15 mm of Hg. All temperatures are reported uncorrected in degrees Celsius (° C.).

In order that this invention may be better understood, the following examples are set forth. These examples are for the purpose of illustration only, and are not to be construed as limiting the scope of the invention in any manner. All publications mentioned herein are incorporated by reference in their entirety.

Analytical LC-MS and UPLC-MS Conditions

LC-MS and UPLC-MS data given in the subsequent specific experimental descriptions refer (unless otherwise noted) to the following conditions:

Method A

Instrument: Waters Acquity UPLC-MS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method B

Instrument: Waters Acquity UPLC-MS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Flash Column Chromatography Conditions

“Purification by (flash) column chromatography” as stated in the subsequent specific experimental descriptions refers to the use of a Biotage Isolera purification system. For technical specifications see “Biotage product catalogue” on www.biotage.com.

General Experimental Procedures

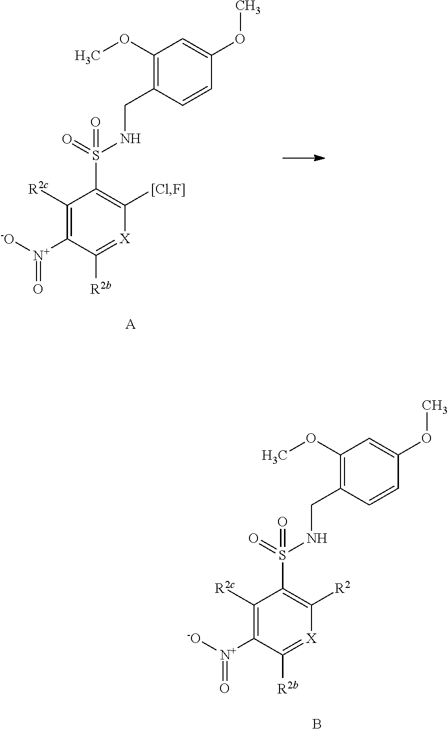

General Procedure GP1.2

Sulfonamide A (e.g. 1.29 mmol) was dissolved in acetonitrile (15 mL in case of 1.29 mmol scale) and finely powdered potassium carbonate (3.0 eq) and the corresponding azole (1.5 eq) were added. Stirring was continued at 100-110° C. until TLC showed consumption of starting material. The solvent was removed under reduced pressure, followed by addition of water and dichloromethane. Afterwards, the phases were separated, the organic phase was dried and it was concentrated in vacuo. The crude was either used without further purification or purified as indicated in the examples.

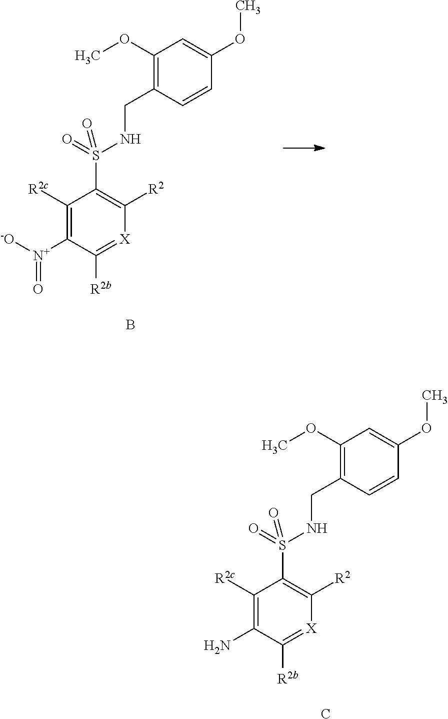

General Procedure GP2.1

Crude nitro compound B (e.g. 1.29 mmol) was dissolved in dioxane (15 mL in case of 1.29 mmol scale) and tin(II)chloride dihydrate (3.0 eq) was added and the reaction mixture was stirred for 2 h at 70° C. After cooling to room temperature the reaction mixture was filtered and concentrated in vacuo. The filtrate was either used without further purification or purified as indicated in the examples.

General Procedure GP2.2

Crude nitro compound B (e.g. 1.29 mmol) was dissolved in dioxane (15 mL in case of 1.29 mmol scale) and tin(II)chloride dihydrate (5.0 eq) was added and the reaction mixture was stirred for 2 h at 70° C. After cooling to room temperature the reaction mixture was filtered and concentrated in vacuo. The filtrate was either used without further purification or purified as indicated in the examples.

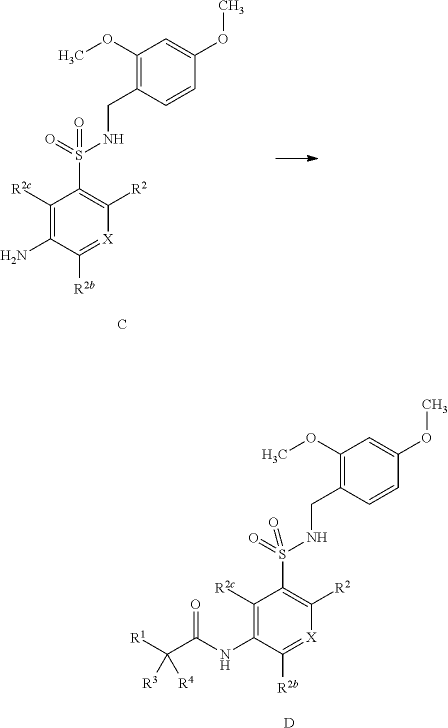

General Procedure GP3.4

Crude substituted aniline C (1.29 mmol) was dissolved in dimethylformamide (10 mL in case of 1.29 mmol scale) followed by the addition of the corresponding acid (amount as indicated in examples), N,N-diisopropylethylamine (2.0 eq based on acid) and HATU (1.0 eq based on acid). The reaction mixture was either stirred overnight at room temperature or heated at 50° C. until TLC showed consumption of starting material. After cooling to room temperature the reaction mixture was concentrated in vacuo. Ethyl acetate and water were added, the organic phase was dried and concentrated in vacuo. The crude was used without further purification.

General Procedure GP3.5

Crude substituted aniline C (1.29 mmol) was dissolved in dimethylformamide (10 mL in case of 1.29 mmol scale) followed by the addition of the corresponding acid (amount as indicated in examples), N,N-diisopropylethylamine (4.0 eq based on acid) and HATU (1.3 eq based on acid). The reaction mixture was either stirred overnight at room temperature or heated at 50° C. until TLC showed consumption of starting material. After cooling to room temperature the reaction mixture was concentrated in vacuo. Ethyl acetate and water were added, the organic phase was dried and concentrated in vacuo. The crude was used without further purification.

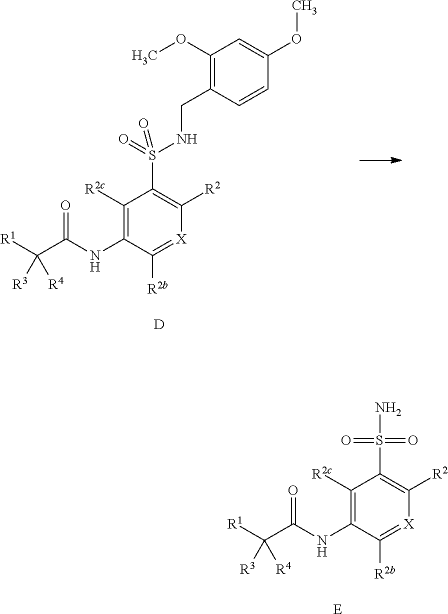

General Procedure GP4.1

Crude amide D (e.g. 1.29 mmol) was dissolved in dichloromethane (5-10 mL in case of 1.29 mmol scale), trifluoroacetic acid (50 eq) was added and the reaction mixture was stirred at room temperature until TLC showed consumption of starting material. The reaction mixture was concentrated in vacuo, ethyl acetate and water were added to the crude and the organic phase was dried and the solvent was removed under reduced pressure. The resulting residue was purified as indicated in the examples. Purification without aqueous extraction was also possible but made the HPLC purification more difficult.

General Procedure GP4.2

Crude amide D (e.g. 1.29 mmol) was dissolved in dichloromethane/trifluoroacetic acid 2/1 (6 mL in case of 1.29 mmol scale) and the reaction mixture was stirred at room temperature until TLC showed consumption of starting material. The reaction mixture was concentrated in vacuo, ethyl acetate and water were added to the crude and the organic phase was dried and the solvent was removed under reduced pressure. The resulting residue was purified as indicated in the examples. Purification without aqueous extraction was also possible but made the HPLC purification more difficult.

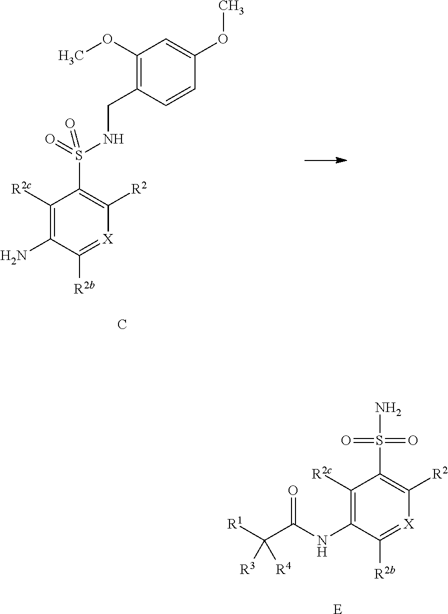

General Procedure GP5.1

Solutions of substituted aniline C (0.20 mmol in 0.4 mL 1-methyl-2-pyrrolidon), the corresponding acid (0.40 mmol in 0.8 mL 1-methyl-2-pyrrolidon), HATU (0.40 mmol in 0.8 mL 1-methyl-2-pyrrolidon), N-methylmorpholine (0.80 mmol in 0.267 mL 1-methyl-2-pyrrolidon, containing 2.5% 4-dimethylaminopyridine) were added and shaken overnight. Then, it was concentrated in vacuo and the residue was redissolved in trifluoroacetic acid/dichloromethane 3/1 (2 mL, containing 5% water). The reaction mixture was again shaken overnight, followed by concentration in vacuo and purification by HPLC.

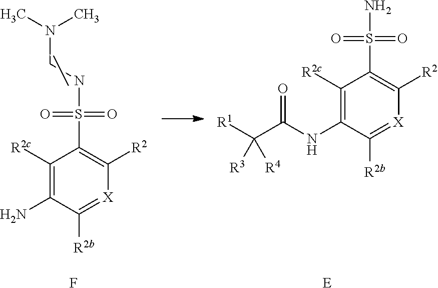

General Procedure GP6.1

Crude substituted aniline F (0.137 mmol) was dissolved in dimethylformamide (2 mL in case of 0.137 mmol scale) followed by the addition of the corresponding acid (amount as indicated in examples), N,N-diisopropylethylamine (2.7 eq based on acid) and HATU (1.0 eq based on acid). The reaction mixture was stirred overnight at room temperature followed by concentration in vacuo. Ethyl acetate and water were added, the organic phase was dried and concentrated in vacuo.

The crude was redissolved in methanol (1 mL), treated with concentrated aqueous ammonia (70 μL) and stirred overnight. The reaction mixture was concentrated in vacuo and purified as indicated in the examples.

Synthesis of Intermediates

### 2-Chloro-N-(2,4-dimethoxybenzyl)-5-nitrobenzenesulfonamide

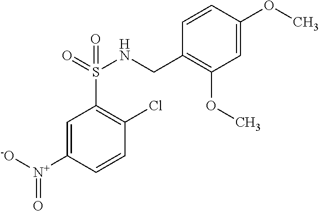

To a solution of 2-chloro-5-nitrobenzenesulfonylchloride (10.8 g, 42.2 mmol) in dichloromethane (108 mL) was added sodium bicarbonate (7.09 g, 84.4 mmol) and 1-(2,4-dimethoxyphenyl)methanamine (7.05 g, 42.2 mmol). The mixture was stirred overnight. The reaction mixture was concentrated in vacuo, followed by addition of water (75 mL) and ethyl acetate (75 mL). After stirring for 10 min the resulting precipitate was separated by filtration and it was dried at 40° C. overnight in vacuo to yield the title compound (14.1 g, 36.5 mmol, 86% yield).

LC-MS (Method A): Rt=1.17 min; MS (ESIneg): m/z=385 [M−H]⁻

¹H-NMR (400 MHz, DMSO-d₆) δ [ppm]: 3.56 (s, 3H), 3.61 (s, 3H), 4.08 (s, 2H), 6.10 (d, 1H), 6.26 (dd, 1H), 7.04 (d, 1H), 7.79 (d, 1H), 8.19 (d, 1H), 8.28 (dd, 1H), 8.45 (s, 1H).

### N-(2,4-Dimethoxybenzyl)-5-nitro-2-[4-(trifluoromethyl)-1H-pyrazol-1-yl]benzenesulfonamide

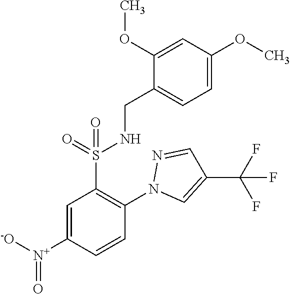

To a solution of 2-chloro-N-(2,4-dimethoxybenzyl)-5-nitrobenzenesulfonamide (5.69 g, 14.7 mmol) in acetonitrile (170 mL) were added 4-(trifluoromethyl)-1H-pyrazole (3.00 g, 22.1 mmol) and powdered potassium carbonate (6.09 g, 44.1 mmol) and it was stirred overnight at 100° C. The reaction mixture was concentrated in vacuo and the residue was extracted with dichloromethane and water. The organic phase was washed with brine and dried over sodium sulfate. Concentration under reduced pressure led to the crude title compound (7.50 g, quant., app. 95% purity) that was used without further purification in the next step.

LC-MS (Method B): Rt=1.31 min; MS (ESIpos): m/z=487 [M+H]⁺

¹H-NMR (400 MHz, DMSO-d₆) δ [ppm]: 3.52 (s, 3H), 3.64 (s, 3H), 4.15 (d, 2H), 6.18 (d, 1H), 6.29 (dd, 1H), 7.08 (d, 1H), 7.93 (d, 1H), 8.03-8.09 (m, 1H), 8.25 (d, 1H), 8.39 (s, 1H), 8.49 (dd, 1H), 8.94 (s, 1H).

### 2-(4-Chloro-1H-pyrazol-1-yl)-N-(2,4-dimethoxybenzyl)-5-nitrobenzenesulfonamide

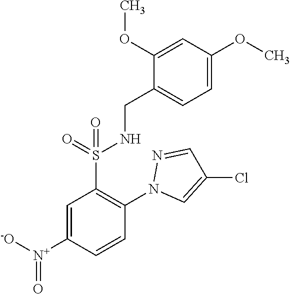

To a solution of 2-chloro-N-(2,4-dimethoxybenzyl)-5-nitrobenzenesulfonamide (5.03 g, 13.0 mmol) in acetonitrile (150 mL) were added 4-chloro-1H-pyrazole (2.00 g, 19.5 mmol) and powdered potassium carbonate (5.39 g, 39.0 mmol) and it was stirred overnight at 100° C. The reaction mixture was concentrated in vacuo and the residue was extracted with dichloromethane and water. The organic phase was washed with brine and dried. Concentration in vacuo led to the crude title compound (6.27 g, quant., app. 95% purity) that was used without further purification in the next step.

LC-MS (Method A): Rt=1.26 min; MS (ESIpos): m/z=453 [M+H]⁺

¹H-NMR (400 MHz, DMSO-d₆) δ [ppm]: 3.48 (s, 3H), 3.62 (s, 3H), 4.15 (s, 2H), 6.14 (d, 1H), 6.27 (dd, 1H), 7.08 (d, 1H), 7.84 (d, 1H), 8.05 (s, 1H), 8.09 (d, 1H), 8.21 (d, 1H), 8.45 (dd, 1H), 8.57 (s, 1H).

### N-(2,4-Dimethoxybenzyl)-2-(4-fluoro-1H-pyrazol-1-yl)-5-nitrobenzenesulfonamide

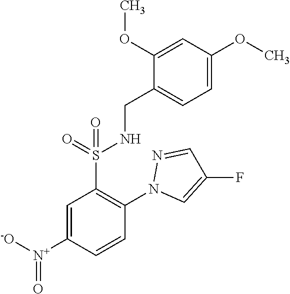

To a solution of 2-chloro-N-(2,4-dimethoxybenzyl)-5-nitrobenzenesulfonamide (5.00 g, 11.6 mmol) in acetonitrile (135 mL) were added 4-fluoro-1H-pyrazole (1.50 g, 17.4 mmol) and powdered potassium carbonate (4.82 g, 34.9 mmol) and it was stirred overnight at 100° C. The reaction mixture was concentrated in vacuo and the residue was extracted with dichloromethane and water. The organic phase was washed with brine and dried over sodium sulfate. Concentration in vacuo led to the crude title compound (5.54 g, quant., app. 85% purity) that was used without further purification in the next step.

LC-MS (Method A): Rt=1.23 min; MS (ESIpos): m/z=437 [M+H]⁺

¹H-NMR (400 MHz, DMSO-d₆) δ [ppm]: 3.48 (s, 3H), 3.62 (s, 3H), 4.13 (s, 2H), 6.15 (d, 1H), 6.28 (dd, 1H), 7.09 (d, 1H), 7.81 (d, 1H), 8.00-8.10 (m, 2H), 8.23 (d, 1H), 8.43 (dd, 1H), 8.59 (s, 1H).

### 2-(4-Bromo-1H-pyrazol-1-yl)-N-(2,4-dimethoxybenzyl)-5-nitrobenzenesulfonamide

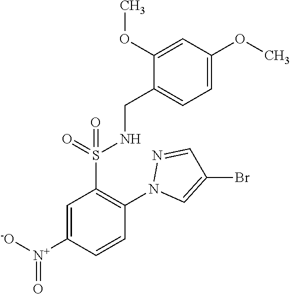

To a solution of 2-chloro-N-(2,4-dimethoxybenzyl)-5-nitrobenzenesulfonamide (1.75 g, 4.54 mmol) in acetonitrile (53 mL) were added 4-bromo-1H-pyrazole (1.00 g, 6.80 mmol) and powdered potassium carbonate (1.88 g, 13.6 mmol) and it was stirred overnight at 100° C. The reaction mixture was concentrated in vacuo and the residue was extracted with dichloromethane and water. The organic phase was washed with brine and dried over sodium sulfate. Concentration in vacuo led to the crude title compound (2.38 g, quant., app. 95% purity) that was used without further purification in the next step.

LC-MS (Method A): Rt=1.29 min; MS (ESIpos): m/z=497 [M+H]⁺

¹H-NMR (400 MHz, DMSO-d₆) δ [ppm]: 3.48 (s, 3H), 3.62 (s, 3H), 4.13 (s, 2H), 6.15 (d, 1H), 6.28 (dd, 1H), 7.09 (d, 1H), 7.84 (d, 1H), 8.00-8.10 (m, 2H), 8.23 (s, 1H), 8.43 (dd, 1H), 8.65 (s, 1H).

### 2-(4-Cyano-1H-pyrazol-1-yl)-N-(2,4-dimethoxybenzyl)-5-nitrobenzenesulfonamide

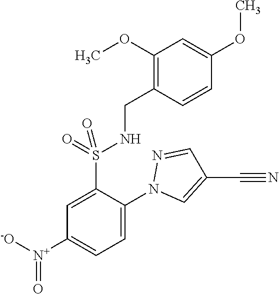

To a solution of 2-chloro-N-(2,4-dimethoxybenzyl)-5-nitrobenzenesulfonamide (15.0 g, 38.8 mmol) in acetonitrile (450 mL) were added 1H-pyrazole-4-carbonitrile (5.41 g, 93.1 mmol) and powdered potassium carbonate (16.1 g, 116 mmol) and it was stirred overnight at 100° C. The reaction mixture was concentrated in vacuo and the residue was extracted with ethyl acetate and water. Pure title compound precipitated and was filtered off (9.09 g 20.5 mmol, 53% yield, 97% purity), The organic phase was washed with brine and dried over sodium sulfate. Concentration in vacuo led to further crude title compound (9.11 g, app. 60% purity).

LC-MS (Method B): Rt=1.17 min; MS (ESIneg): m/z=442 [M−H]⁻

¹H-NMR (400 MHz, DMSO-d₆) δ [ppm]: 3.53 (s, 3H), 3.64 (s, 3H), 4.08 (s, 2H), 6.20 (d, 1H), 6.29 (dd, 1H), 7.07 (d, 1H), 7.89 (d, 1H), 8.12 (br s, 1H), 8.30 (br s, 1H), 8.41-8.54 (m, 2H), 9.17 (br s, 1H).

### 5-Amino-N-(2,4-dimethoxybenzyl)-2-[4-(trifluoromethyl)-1H-pyrazol-1-yl]benzenesulfonamide

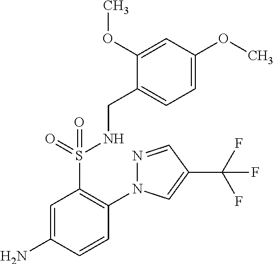 [SMILES: COc1ccc(CNS(=O)(=O)c2cc(N)ccc2-n2cc(C(F)(F)F)cn2)c(OC)c1]

Pd/C (10% loading, 750 mg) was added to a solution of N-(2,4-dimethoxybenzyl)-5-nitro-2-[4-(trifluoromethyl)-1H-pyrazol-1-yl]benzenesulfonamide (7.50 g, 14.7 mmol) in methanol (120 mL) and stirred under a hydrogen atmosphere for 4 h at room temperature.

Some ethyl acetate was added to dissolve precipitated product, followed by filtration, washing and concentration in vacuo to give the crude title compound (6.50 g, quant., app. 95% purity) that was used without further purification in the next step.

LC-MS (Method A): Rt=1.20 min; MS (ESIpos): m/z=457 [M+H]⁺

¹H-NMR (400 MHz, DMSO-d₆) δ [ppm]: 3.70 (s, 3H), 3.73 (s, 3H), 3.94 (d, 2H), 6.01 (s, 2H), 6.41-6.48 (m, 2H), 6.78 (dd, 1H), 7.09-7.14 (m, 2H), 7.18-7.27 (m, 2H), 8.12 (s, 1H), 8.56 (s, 1H).

### 5-Amino-2-(4-chloro-1H-pyrazol-1-yl)-N-(2,4-dimethoxybenzyl)benzenesulfonamide

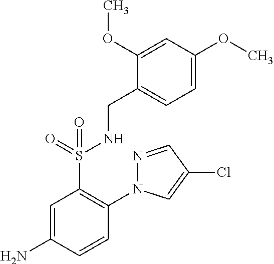 [SMILES: COc1ccc(CNS(=O)(=O)c2cc(N)ccc2-n2cc(Cl)cn2)c(OC)c1]

Pt/C (10% loading, 600 mg) was added to a solution of crude 2-(4-chloro-1H-pyrazol-1-yl)-N-(2,4-dimethoxybenzyl)-5-nitrobenzenesulfonamide (6.27 g, 13.9 mmol) in ethanol (100 mL) and stirred under a hydrogen atmosphere for 24 h at room temperature. The catalyst was filtered off, washed with ethyl acetate and the filtrate was concentrated in vacuo to give the crude title compound (5.99 g, quant., app. 90% purity) that was used without further purification in the next step.

LC-MS (Method A): Rt=1.23 min; MS (ESIpos): m/z=423 [M+H]⁺

¹H-NMR (400 MHz, DMSO-d₆) δ [ppm]: 3.69 (s, 3H), 3.72 (s, 3H), 3.92 (d, 2H), 5.95 (s, 2H), 6.41-6.47 (m, 2H), 6.76 (dd, 1H), 7.08-7.12 (m, 2H), 7.15 (d, 1H), 7.19 (t, 1H), 7.78 (d, 1H), 8.15 (d, 1H).

### 5-Amino-N-(2,4-dimethoxybenzyl)-2-(4-fluoro-1H-pyrazol-1-yl)benzenesulfonamide

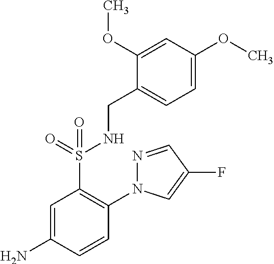 [SMILES: COc1ccc(CNS(=O)(=O)c2cc(N)ccc2-n2cc(F)cn2)c(OC)c1]

Pt/C (10% loading, 1.76 g) was added to a solution of crude N-(2,4-dimethoxybenzyl)-2-(4-fluoro-1H-pyrazol-1-yl)-5-nitrobenzenesulfonamide (5.50 g, 12.6 mmol) in a mixture of ethanol (125 mL) and dioxane (200 mL) and stirred under a hydrogen atmosphere for 8 h at room temperature. The catalyst was filtered off, washed with ethyl acetate and the filtrate was concentrated in vacuo to give the crude title compound (5.07 g, quant., app. 90% purity) that was used without further purification in the next step.

LC-MS (Method A): Rt=1.10 min

MS (ESIpos): m/z=407 [M+H]⁺

¹H-NMR (400 MHz, DMSO-d₆) δ [ppm]: 3.69 (s, 3H), 3.72 (s, 3H), 3.92 (d, 2H), 5.93 (s, 2H), 6.42-6.47 (m, 2H), 6.78 (dd, 1H), 7.08-7.19 (m, 4H), 7.74 (dd, 1H), 8.07 (dd, 1H).

### 5-Amino-2-(4-bromo-1H-pyrazol-1-yl)-N-(2,4-dimethoxybenzyl)benzenesulfonamide

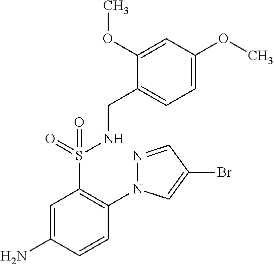 [SMILES: COc1ccc(CNS(=O)(=O)c2cc(N)ccc2-n2cc(Br)cn2)c(OC)c1]

Pt/C (10% loading, 1.76 g) was added to a solution of crude 2-(4-bromo-1H-pyrazol-1-yl)-N-(2,4-dimethoxybenzyl)-5-nitrobenzenesulfonamide (5.60 g, 12.8 mmol) in ethanol (140 mL) and stirred under a hydrogen atmosphere for 14 h at room temperature. The catalyst was filtered off, washed with ethyl acetate and the filtrate was concentrated in vacuo to give the crude title compound (1.87 g, quant., app. 90% purity) that was used without further purification in the next step.

LC-MS (Method A): Rt=1.18 min; MS (ESIpos): m/z=467 (M+H)⁺

¹H-NMR (400 MHz, DMSO-d₆) δ [ppm]: 3.69 (s, 3H), 3.72 (s, 3H), 3.92 (d, 2H), 5.95 (s, 2H), 6.39-6.48 (m, 2H), 6.77 (dd, 1H), 7.08-7.23 (m, 4H), 7.79 (d, 1H), 8.15 (d, 1H).

### 2-Chloro-N-[(dimethylamino)methylene]-5-nitrobenzenesulfonamide

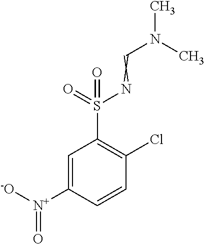

1,1-Dimethoxy-N,N-dimethylmethanamine (3.02 g, 25.4 mmol) was added to a solution of 2-chloro-5-nitrobenzenesulfonamide (3.00 g, 12.7 mmol) in N,N-dimethylformamide (43 mL) and was stirred at room temperature for 2 days. The reaction mixture was concentrated in vacuo and the residue was extracted with dichloromethane/water. The organic phase was washed with brine and dried. Concentration in vacuo gave the crude title compound (4.18 g, quant., app. 90% purity) that was used without further purification in the next step.

LC-MS (Method A): Rt=0.86 min; MS (ESIpos): m/z=292 [M+H]⁺

¹H-NMR (400 MHz, DMSO-d₆) δ [ppm] 2.94-2.96 (m, 3H), 3.20 (s, 3H), 7.91 (d, 1H), 8.31-8.33 (m, 1H), 8.39 (dd, 1H), 8.69 (d, 1H).

### N-[(Dimethylamino)methylene]-5-nitro-2-[5-(trifluoromethyl)pyridin-3-yl]benzenesulfonamide

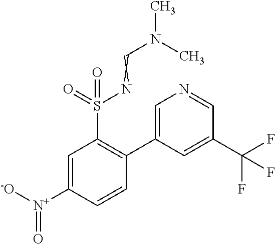

2-Chloro-N-[(dimethylamino)methylene]-5-nitrobenzenesulfonamide (1.10 g, 3.77 mmol) was dissolved in degassed n-propanol (33 mL) and treated with [5-(trifluoromethyl)pyridin-3-yl]boronic acid (1.08 g, 5.68 mmol), bis(triphenylphosphine)palladium(II) dichloride (132 mg, 0.189 mmol) and triphenylphosphine (49.5 mg, 0.189 mmol). Aqueous degassed 2M potassium carbonate solution (5.65 mL) was added, the vial was sealed and stirred for 16 hours at 100° C. After cooling to room temperature water was added and it was extracted three times with ethyl acetate followed by concentration in vacuo.

The partly deprotected target molecule was reprotected as previously described by stirring at room temperature with 1,1-dimethoxy-N,N-dimethylmethanamine in NDMF. The reaction mixture was concentrated in vacuo and the residue was purified by preparative HPLC (Chromatorex C-18 10 μm, 125×30 mm, acetonitrile/water+0.1% aqueous ammonia (32%)) to give the title compound (174 mg, 0.432 mmol, 11% yield, 95% purity).

LC-MS (Method B): Rt=1.08 min; MS (ESIpos): m/z=403 [M+H]⁺

¹H-NMR (400 MHz, DMSO-d₆) δ [ppm] 2.76 (s, 3H), 2.99 (s, 3H), 7.76 (s, 1H), 7.81 (d, 1H), 8.33-8.36 (m, 1H), 8.52 (dd, 1H), 8.76 (d, 1H), 8.88 (d, 1H), 9.09 (dd, 1H).

### 5-Amino-N-[(dimethylamino)methylene]-2-[5-(trifluoromethyl)pyridin-3-yl]benzenesulfonamide

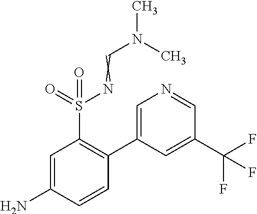 [SMILES: CN(C)C=NS(=O)(=O)c1cc(N)ccc1-c1cncc(C(F)(F)F)c1]

Pd/C (10% loading, 21 mg) was added to a solution of N-[(dimethylamino)methylene]-5-nitro-2-[5-(trifluoromethyl)pyridin-3-yl]benzenesulfonamide (174 mg, 0.39 mmol) in a mixture of methanol (10 mL) and dioxane (10 mL) and stirred under a hydrogen atmosphere overnight at room temperature. The catalyst was filtered off, washed with ethyl acetate and the filtrate was concentrated in vacuo to give the title compound (140 mg, quant., 95% purity) that was used without further purification in the next step.

LC-MS (Method A): Rt=0.90 min; MS (ESIpos): m/z=373 [M+H]⁺

### 2-[1-(Difluoromethyl)-1H-pyrazol-4-yl]-N-[(dimethylamino)methylene]-5-nitrobenzenesulfonamide

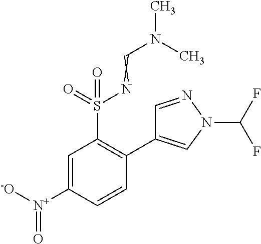

2-Chloro-N-[(dimethylamino)methylene]-5-nitrobenzenesulfonamide (1.00 g, 3.43 mmol) was dissolved in degassed n-propanol (30 mL) and treated with 1-(difluoromethyl)-4-(4,4,5,5-tetramethyl-1,3,2-dioxaborolan-2-yl)-1H-pyrazole (1.25 g, 5.14 mmol), bis(triphenylphosphine)palladium(II) dichloride (121 mg, 0.171 mmol) and triphenylphosphine (45.0 mg, 0.171 mmol). Aqueous degassed 2M potassium carbonate solution (5.14 mL) was added, the vial was sealed and stirred for 16 hours at 100° C. After cooling to room temperature water was added and it was extracted three times with ethyl acetate followed by concentration in vacuo.

The residue was redissolved in a mixture of methanol (25 mL) and n-propanol (25 mL) and concentrated aqueous ammonia (50 mL) was added to completely deprotect the target molecule for easier purification. The reaction mixture was extracted with dichloromethane and ethyl acetate. The organic phases were dried, followed by concentration in vacuo and purification by preparative HPLC (Chromatorex C-18 10 μm, 125×30 mm, acetonitrile/water+0.1% aqueous ammonia (32%)) to give 2-[1-(difluoromethyl)-1H-pyrazol-4-yl]-5-nitrobenzenesulfonamide (383 mg).

Next, the deprotected target molecule was reprotected as previously described by stirring at room temperature with 1,1-dimethoxy-N,N-dimethylmethanamine in DMF. Concentration in vacuo gave the title compound (418 mg) that was used without further purification in the next step.

LC-MS (Method B): Rt=0.98 min; MS (ESIpos): m/z=374 [M+H]⁺

¹H-NMR (400 MHz, DMSO-d₆) δ [ppm] 2.76 (d, 3H), 3.02 (s, 3H), 7.85 (d, 1H), 7.91-7.93 (m, 2H), 7.95 (t, 1H), 8.19-8.21 (m, 1H), 8.43 (dd, 1H), 8.72 (d, 1H), 8.77 (d, 1H).

### 5-Amino-2-[1-(difluoromethyl)-1H-pyrazol-4-yl]-N-[(dimethylamino)methylene]-benzenesulfonamide

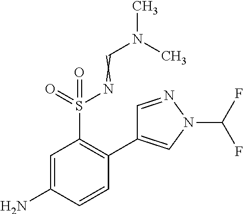 [SMILES: CN(C)C=NS(=O)(=O)c1cc(N)ccc1-c1cnn(C(F)F)c1]

Pd/C (10% loading, 54 mg) was added to a solution of 2-[1-(difluoromethyl)-1H-pyrazol-4-yl]-N-[(dimethylamino)methylene]-5-nitrobenzenesulfonamide (418 mg, 1.01 mmol) in a mixture of methanol (10 mL) and dioxane (10 mL) and stirred under a hydrogen atmosphere overnight at room temperature. The catalyst was filtered off, washed with ethyl acetate and the filtrate was concentrated in vacuo to give the crude title compound (370 mg, quant., 90% purity) that was used without further purification in the next step.

LC-MS (Method A): Rt=0.74 min; MS (ESIpos): m/z=344 [M+H]⁺

### 2-Bromo-5-nitrobenzenesulfonamide

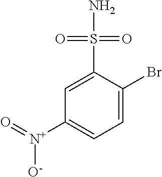 [SMILES: NS(=O)(=O)c1cc([N+](=O)[O-])ccc1Br]

2-Bromo-5-nitrobenzenesulfonyl chloride (20.0 g, 66.6 mmol) was dissolved in 1,4-dioxane (100 ml) and cooled to 0° C. Aqueous ammonia (400 ml, 0.50 M, 200 mmol) was slowly added and stirring was continued at room temperature until completion of the reaction. The solvent was removed under reduced pressure and dichloromethane was added. The organic phase was washed with water three times. The suspension was filtered (solid is product), and the organic phase was washed with brine. The combined organic phases were dried over sodium sulfate and the solvent was removed under reduced pressure. The crude was recrystallized from diethyl ether to yield 16.4 g (93% purity, 88% yield).

LC-MS (Method B): Rt=0.45 min; MS (ESIpos): m/z=281 [M+H]⁺

### 2-Bromo-N-[(dimethylamino)methylidene]-5-nitrobenzenesulfonamide

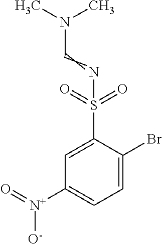 [SMILES: CN(C)C=NS(=O)(=O)c1cc([N+](=O)[O-])ccc1Br]

2-Bromo-5-nitrobenzenesulfonamide (16.4 g, 58.3 mmol) was dissolved in DMF (200 ml) at room temperature and 1,1-dimethoxy-N,N-dimethylmethanamine (15 ml, 120 mmol) was added. Stirring was continued until completion of the reaction. The solvent was removed under reduced pressure and the crude partitioned between dichloromethane and brine. The organic phase was dried over Whatmanfilter and the solvent was removed under reduced pressure. The crude was used in the next step without further purification (19.2 g, 78% purity, 98% yield).

LC-MS (Method A): Rt=0.92 min; MS (ESIpos): m/z=336 [M+H]⁺

### 5-Amino-2-bromo-N-[(dimethylamino)methylidene]benzenesulfonamide

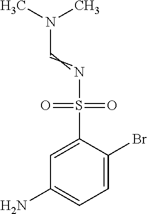 [SMILES: CN(C)C=NS(=O)(=O)c1cc(N)ccc1Br]

2-Bromo-N-[(dimethylamino)methylidene]-5-nitrobenzenesulfonamide (12.7 g, 37.8 mmol) was dissolved in methanol (170 ml) and the flask was flushed with nitrogen. Platinum on charcoal (5% loading, 1.61 g, 8.26 mmol) was added and the flask was evacuated and subsequently flushed with hydrogen (1 bar). Stirring was continued at room temperature until completion of the reaction. The reaction mixture was filtered over Celite and the solvent was removed under reduced pressure. The crude was used without further purification in the next step (5.5 g, 76% purity, 59% yield).

LC-MS (Method A): Rt=0.75 min; MS (ESIpos): m/z=306 [M+H]⁺

### N-(4-Bromo-3-{[(dimethylamino)methylidene]sulfamoyl}phenyl)-2-(2-chlorophenyl)acetamide

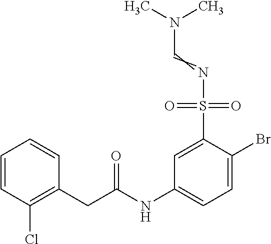 [SMILES: CN(C)C=NS(=O)(=O)c1cc(NC(=O)Cc2ccccc2Cl)ccc1Br]

5-Amino-2-bromo-N-[(dimethylamino)methylidene]benzenesulfonamide (4.85 g, 15.8 mmol) was dissolved in DMF (100 ml) and (2-chlorophenyl)acetic acid (3.24 g, 19.0 mmol) was added followed by the addition of N,N-diisopropylethylamine (13 ml, 79 mmol) and HATU (9.64 g, 25.3 mmol). The reaction mixture was stirred for 3 h at 50° C. The solvent was removed under reduced pressure and ethyl acetate and water were added. The phases were separated and the aqueous phase was extracted with ethyl acetate. The combined organic phases were dried over Whatmanfilter and the solvent was removed under reduced pressure. The crude was suspended in dichloromethane and filtered, the solvent was removed and the crude was used without further purification in the next step (15.7 g).

LC-MS (Method B): Rt=1.08 min; MS (ESIpos): m/z=458 [M+H]⁺

This intermediate can also be used as the HCl salt.

### 5-Amino-2-(1-cyclopropyl-1H-pyrazol-4-yl)-N-[(dimethylamino)methylidene]benzenesulfonamide

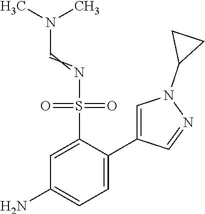 [SMILES: CN(C)C=NS(=O)(=O)c1cc(N)ccc1-c1cnn(C2CC2)c1]

2-Chloro-5-nitrobenzenesulfonamide (674 mg, 2.85 mmol) and 1-cyclopropyl-4-(4,4,5,5-tetramethyl-1,3,2-dioxaborolan-2-yl)-1H-pyrazole (1.00 g, 4.27 mmol) were dissolved in n-propanol (34 ml) and bis(triphenylphosphine)palladium(II) dichloride (CAS 13965-03-2) (100 mg, 142 μmol) and triphenylphosphine (37.3 mg, 142 μmol) were added. The reaction was purged with argon for 5 minutes and aq. potassium carbonate (5.7 ml, 1.0 M, 5.7 mmol) was added. The reaction was heated at 100° C. for 3 h. Afterwards the mixture was filtered over Celite and the solvent was removed under reduced pressure. Ethyl acetate and water were added. The phases were separated and the aqueous phase was extracted with ethyl acetate. The combined organic phases were dried over Whatmanfilter and the solvent was removed under reduced pressure. The crude was used in the next step without further purification.

2-(1-Cyclopropyl-1H-pyrazol-4-yl)-5-nitrobenzenesulfonamide (1.17 g, 3.79 mmol) and 1,1-dimethoxy-N,N-dimethylmethanamine (1.0 ml, 7.6 mmol) were dissolved in DMF (25 ml) and the reaction was stirred at room temperature until completion of the reaction. The solvent was removed under reduced pressure and the crude was used without further purification in the next step.

2-(1-Cyclopropyl-1H-pyrazol-4-yl)-N-[(dimethylamino)methylidene]-5-nitrobenzenesulfonamide (1.84 g, 5.06 mmol) was dissolved in THE (30 ml) and the flask was flushed with nitrogen. Palladium on charcoal (10% loading, 53.9 g, 506 μmol) was added and the flask was evacuated and subsequently flushed with hydrogen (1 bar). Stirring was continued at room temperature until completion of the reaction. The reaction mixture was filtered over Celite and the solvent was removed under reduced pressure.

The crude was used without further purification in the next step (1.3 g, 53% purity, 75% yield over 3 steps).

LC-MS (Method A): Rt=0.70 min; MS (ESIpos): m/z=334 [M+H]⁺

### 5-Amino-2-[1-(difluoromethyl)-1H-pyrazol-4-yl]benzenesulfonamide

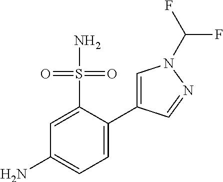 [SMILES: Nc1ccc(-c2cnn(C(F)F)c2)c(S(N)(=O)=O)c1]

2-Bromo-N-[(dimethylamino)methylidene]-5-nitrobenzenesulfonamide (800 mg, 2.3 mmol) and 1-(difluoromethyl)-4-(4,4,5,5-tetramethyl-1,3,2-dioxaborolan-2-yl)-1H-pyrazole (700 mg, 2.87 mmol) were dissolved in n-propanol (15 ml) and bis(triphenyl-phosphine)palladium(II) dichloride (CAS 13965-03-2) (84 mg, 119 μmol) and triphenylphosphine (31 mg, 119 μmol) were added. The solution was purged with argon for 5 minutes and aq. potassium carbonate (3.6 ml, 2.0 M, 7.2 mmol) was added. The reaction was heated at 100° C. for 16 h. Water and ethyl acetate were added. The phases were separated and the aqueous phase was extracted with ethyl acetate. The combined organic phases were dried over Whatmanfilter and the solvent was removed under reduced pressure. The crude was used in the next step without further purification.

2-[1-(Difluoromethyl)-1H-pyrazol-4-yl]-N-[(dimethylamino)methylidene]-5-nitrobenzenesulfonamide (2.16 g, 5.79 mmol) was dissolved in tetrahydrofurane (50 ml) and platinum on charcoal (5% loading, 307 mg, 1.57 mmol) was added. The flask was evacuated three times and flushed with hydrogen (1 bar). The reaction was stirred for 4 h at room temperature. According to UPLC-MS the reaction was not complete and same amounts of platinum on charcoal were added and the reaction was stirred under hydrogen atmosphere for further 16 h. Afterwards, the mixture was filtered over Celite and the solvent was removed under reduced pressure. The crude was taken to the next step without further purification.

5-Amino-2-[1-(difluoromethyl)-1H-pyrazol-4-yl]-N-[(dimethylamino)methylidene]benzenesulfonamide (670 mg, 1.95 mmol) was dissolved in methanol (25 ml) and treated with 25% aqueous ammonia solution (25 ml) at room temperature until completion of the reaction. The solvent was removed under reduced pressure and the crude was purified by chromatography on silica gel (Biotage, gradient dichloromethane/ethyl acetate) and subsequent HPLC purification ((Waters XBrigde C18 5p 100×30 mm, acetonitrile/water+0.1% formic acid) to yield 53 mg (99% purity, 9% yield over 3 steps). The reactions were repeated and the crude was used for the next steps using this intermediate.

LC-MS (Method B): Rt=0.58 min; MS (ESIpos): m/z=289 [M+H]⁺

### 5-Bromo-2-chloro-N-[(dimethylamino)methylidene]pyridine-3-sulfonamide

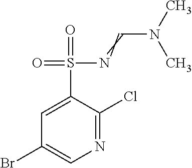 [SMILES: CN(C)C=NS(=O)(=O)c1cc(Br)cnc1Cl]

5-Bromo-2-chloropyridine-3-sulfonamide (3.86 g, 14.2 mmol) and 1,1-dimethoxy-N,N-dimethylmethanamine (3.8 ml, 28 mmol) were dissolved in DMF (40 ml) and stirred for 2 h at room temperature. The solvent was removed under reduced pressure and dichloromethane and brine were added. The phases were separated and the organic phase was washed with water. The combined organic phases were dried over Whatmanfilter and the solvent was removed under reduced pressure. The crude was used in the next step without further purification (5.12 g).

LC-MS (Method B): Rt=0.89 min; MS (ESIpos): m/z=326 [M+H]⁺

### 2-Chloro-N-[(dimethylamino)methylidene]-5-[(diphenylmethylidene)amino]pyridine-3-sulfonamide

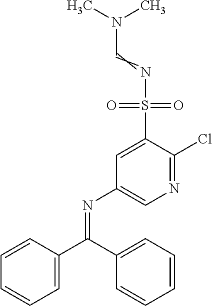 [SMILES: CN(C)C=NS(=O)(=O)c1cc(N=C(c2ccccc2)c2ccccc2)cnc1Cl]

5-Bromo-2-chloro-N-[(dimethylamino)methylidene]pyridine-3-sulfonamide (5.00 g, 15.3 mmol), 1,1-diphenylmethanimine (3.9 ml, 23 mmol), XantPhos (886 mg, 1.53 mmol) and palladium(II) acetate (172 mg, 765 μmol) were dissolved in dioxane (150 ml). The solution was purged with argon for 5 minutes and cesium carbonate (15.0 g, 45.9 mmol) was added. The reaction was heated at 100° C. for 1 h, Afterwards, the solvent was removed under reduced pressure and water and ethyl acetate were added. The phases were separated and the aqueous phase was extracted with ethyl acetate. The combined organic phases were dried over Whatmanfilter and the solvent was removed under reduced pressure. Half of the crude was used without further purification and 3 g were purified by chromatography on ammonia coated silica gel (Biotage, hexane/ethyl acetate) to yield 1.00 g (78% purity, 15% yield based on total amount of starting material) LC-MS (Method B): Rt=1.26 min; MS (ESIpos): m/z=427 [M+H]⁺

### 2-[1-(Difluoromethyl)-1H-pyrazol-4-yl]-N-[(dimethylamino)methylidene]-5-[(diphenyl-methylidene)amino]pyridine-3-sulfonamide

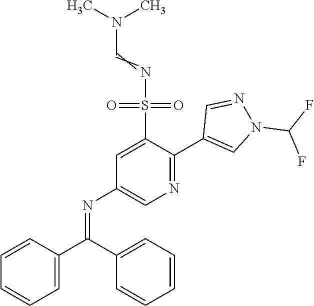 [SMILES: CN(C)C=NS(=O)(=O)c1cc(N=C(c2ccccc2)c2ccccc2)cnc1-c1cnn(C(F)F)c1]

2-Chloro-N-[(dimethylamino)methylidene]-5-[(diphenylmethylidene)amino]pyridine-3-sulfonamide (1.50 g, 3.51 mmol, crude) and 1-(difluoromethyl)-4-(4,4,5,5-tetramethyl-1,3,2-dioxaborolan-2-yl)-1H-pyrazole (1.71 g, 7.03 mmol) were dissolved in n-propanol (30 ml)/DMF (15 ml) and bis(triphenylphosphine)palladium(II) dichloride (CAS 13965-03-2) (371 mg, 527 μmol), triphenylphosphine (225 mg, 0.85 mmol), potassium fluoride (408 mg, 7.03 mmol) and aq. potassium phosphate solution (1.8 ml, 2.0 M, 3.5 mmol) were added. The solution was purged with argon for 5 minutes and the reaction was heated at 100° C. for 1 h in the microwave (1 bar/30 W). The solvent was removed under reduced pressure and water and ethyl acetate were added. The phases were separated and the aqueous phase was extracted with ethyl acetate. The combined organic phases were dried over Whatmanfilter and the solvent was removed under reduced pressure. The crude was purified by chromatography on ammonia coated silica gel (Biotage, hexane/ethyl acetate)(1.54 g, 65% purity, 86% yield).

LC-MS (Method B): Rt=1.26 min; MS (ESIpos): m/z=509 [M+H]⁺

### 5-Amino-2-[1-(difluoromethyl)-1H-pyrazol-4-yl]-N-[(dimethylamino)methylidene]pyridine-3-sulfonamide

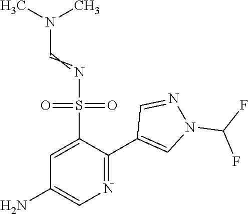 [SMILES: CN(C)C=NS(=O)(=O)c1cc(N)cnc1-c1cnn(C(F)F)c1]

2-[1-(Difluoromethyl)-1H-pyrazol-4-yl]-N-[(dimethylamino)methylidene]-5-[(diphenylmethylidene)amino]pyridine-3-sulfonamide (1.54 g, 3.03 mmol) was dissolved in dioxane (15 ml) and aq. HCl (2.0 ml, 3.0 M, 6.1 mmol) was added. The reaction was stirred for 1 h at room temperature. The solvent was removed under reduced pressure and the crude was used without further purification in the next step (2.45 g).

LC-MS (Method B): Rt=0.66 min; MS (ESIpos): m/z=345 [M+H]⁺

### 5-Amino-2-(4-chloro-1H-pyrazol-1-yl)-N-[(dimethylamino)methylene]pyridine-3-sulfonamide

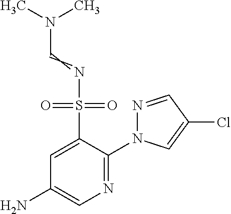 [SMILES: CN(C)C=NS(=O)(=O)c1cc(N)cnc1-n1cc(Cl)cn1]

2-Chloro-N-[(dimethylamino)methylene]-5-[(diphenylmethylene)amino]pyridine-3-sulfonamide (1.00 g, 2.34 mmol) was dissolved in DMSO (18 mL). 4-Chloro-1H-pyrazole (480 mg, 4.69 mmol), potassium iodide (389 mg, 2.34 mmol) and potassium phosphate (746 mg, 3.51 mmol) were added and the reaction mixture was stirred overnight at 100° C. Afterwards it was concentrated in vacuo, extracted with dichloromethane/water and the organic phase was washed with brine and dried over sodium sulfate followed by concentration in vacuo.

Due to partial deprotection, the material was redissolved in DMF (2 mL) and stirred overnight with 1,1-dimethoxy-N,N-dimethylmethanamine (0.5 mL). Stirring overnight resulted in a precipitate that was removed by filtration (229 mg pure 2-(4-chloro-1H-pyrazol-1-yl)-N-[(dimethylamino)methylene]-5-[(diphenylmethylene)amino]pyridine-3-sulfonamide). The filtrate was concentrated in vacuo, extracted with dichloromethane/water and the organic phase was washed with brine and dried over sodium sulfate followed by concentration in vacuo to give crude 2-(4-chloro-1H-pyrazol-1-yl)-N-[(dimethylamino)methylene]-5-[(diphenylmethylene)amino]pyridine-3-sulfonamide (549 mg).

LC-MS (Method A): Rt=1.31 min, MS (ESIpos): m/z=493 [M+H]⁺

¹H-NMR (400 MHz, DMSO-d₆) δ [ppm] 2.80 (s, 3H), 3.09 (s, 3H), 7.27-7.33 (m, 2H), 7.38-7.44 (m, 3H), 7.49-7.56 (m, 2H), 7.58-7.64 (m, 1H), 7.67 (s, 1H), 7.69-7.76 (m, 2H), 7.79-7.83 (m, 2H), 8.17 (d, 1H), 8.32 (d, 1H).

The pure material (229 mg) from the previous step was dissolved in dioxane (2.0 mL) and 2M HCl in dioxane (1.00 mL, 2.00 mmol) was added, followed by stirring overnight. It was concentrated in vacuo and extracted with ethyl acetate/water. The organic phase was washed with brine, dried over sodium sulfate and concentrated in vacuo to yield the crude title compound (200 mg) that was used without further purification in the next steps.

LC-MS (Method A): Rt=0.71 min, MS (ESIpos): m/z=329 [M+H]⁺

### 5-Amino-N-[(dimethylamino)methylidene]-2-[4-(trifluoromethyl)-1H-pyrazol-1-yl]-pyridine-3-sulfonamide

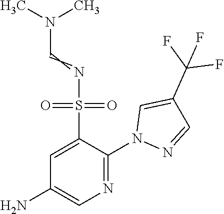 [SMILES: CN(C)C=NS(=O)(=O)c1cc(N)cnc1-n1cc(C(F)(F)F)cn1]

The reaction was carried out on a three times 1 g scale. 2-Chloro-N-[(dimethylamino)methylidene]-5-[(diphenylmethylidene)amino]pyridine-3-sulfonamide (3.00 g, 7.03 mmol) and 4-(trifluoromethyl)-1H-pyrazole (1.43 g, 10.5 mmol) were dissolved in DMSO (110 ml, 1.6 mol) and potassium iodide (583 mg, 3.51 mmol) and potassium phosphate (2.24 g, 10.5 mmol) were added. The reaction was heated for 5 h in the microwave at 100° C. Afterwards, the solid was filtered off and to the filtrate ethyl acetate and water were added. The organic phase was washed with brine and dried over sodium sulfate. The solvent was removed under reduced pressure and the crude was purified by chromatography on silica gel (Biotage, ethyl atecate/hexane) to yield 15.7 g (424% yield).

LC-MS (Method A): Rt=1.40 min; MS (ESIpos): m/z=472 [M+H]⁺

5-[(Diphenylmethylidene)amino]-2-[4-(trifluoromethyl)-1H-pyrazol-1-yl]pyridine-3-sulfonamide (3.50 g, 7.42 mmol) was dissolved in 1,4-dioxane (100 ml) and HCl (4.9 ml, 3.0 M, 15 mmol) was added. The reaction was stirred at room temperature for 2 h. The solvent was removed under reduced pressure and the crude was partitioned between ethyl acetate and water. Afterwards, the organic phase was dried over Whatmanfilter and the solvent was removed under reduced pressure. The crude was dissolved in acetonitrile and water and lyophilized over night.

LC-MS (Method B): Rt=0.56 min; MS (ESIpos): m/z=307 [M+H]⁺

### 2-(4-Cyano-1H-pyrazol-1-yl)-5-nitrobenzenesulfonamide

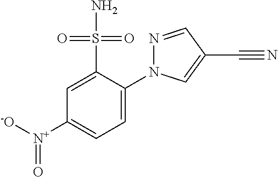

2-Chloro-5-nitrobenzenesulfonamide (250 mg, 1.06 mmol) was dissolved in acetonitrile (10 mL), followed by addition of 1H-pyrazole-4-carbonitrile (148 mg, 1.59 mmol) and finely powdered potassium carbonate (438 mg, 3.17 mmol). The reaction mixture was stirred overnight at 100° C. After cooling to room temperature dichloromethane and water were added and the organic phase was washed with brine solution, dried over sodium sulfate and concentrated in vacuo. Purification by preparative HPLC (Chromatorex C-18 10 μm, 125×30 mm, acetonitrile/water+0.1% formic acid) gave the title compound (128 mg, 0.436 mmol, 41% yield, 70% purity).

LC-MS (Method A): Rt=0.78 min; MS (ESIpos): m/z=294 [M+H]⁺

¹H-NMR (400 MHz, DMSO-d₆) δ [ppm]: 7.94 (br d, 2H), 7.98 (d, 1H), 8.42 (d, 1H), 8.61 (dd, 1H), 8.83 (d, 1H), 9.04 (d, 1H).

### 5-Amino-2-(4-cyano-1H-pyrazol-1-yl)benzenesulfonamide

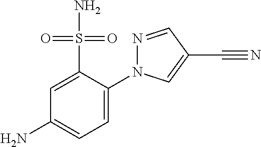 [SMILES: N#Cc1cnn(-c2ccc(N)cc2S(N)(=O)=O)c1]

2-(4-Cyano-1H-pyrazol-1-yl)-5-nitrobenzenesulfonamide (128 mg, 0.44 mmol) was dissolved in methanol (17 mL) and dioxane (3 mL). The flask was evacuated and flushed with nitrogen, followed by the addition of palladium on carbon (13 mg, 10% loading). It was again evacuated and now flushed with hydrogen, followed by stirring under a hydrogen atmosphere for 5 h at room temperature. The hydrogen was removed, the catalyst filtered off and the filtrate was concentrated in vacuo. It was redissolved in dichloromethane and again concentrated in vacuo to give the title compound (81 mg, 0.308 mmol, 70% yield, 79% purity).

LC-MS (Method B): Rt=0.46 min; MS (ESIpos): m/z=264 [M+H]⁺

¹H-NMR (400 MHz, DMSO-d₆) δ [ppm]: 6.06 (s, 2H), 6.77 (dd, 1H), 7.17-7.23 (m, 4H), 8.23 (d, 1H), 8.71 (d, 1H).

### SYNTHESIS OF EXAMPLES

### Example 19

### 2-(2-Chlorophenyl)-N-{3-sulfamoyl-4-[4-(trifluoromethyl)-1H-pyrazol-1-yl]phenyl}acetamide

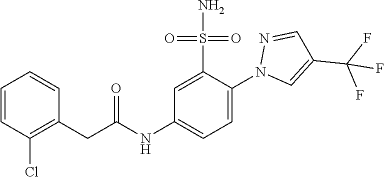 [SMILES: NS(=O)(=O)c1cc(NC(=O)Cc2ccccc2Cl)ccc1-n1cc(C(F)(F)F)cn1]

5-Amino-N-(2,4-dimethoxybenzyl)-2-[4-(trifluoromethyl)-1H-pyrazol-1-yl]benzenesulfonamide (22.3 g, 48.9 mmol) was dissolved in DMF (460 mL) followed by the addition of (2-chlorophenyl)acetic acid (12.5 g, 73.3 mmol), N,N-diisopropylethylamine (25.3 g, 195 mmol) and HATU (27.9 g, 73.3 mmol). The reaction mixture was stirred overnight at room temperature. It was then concentrated in vacuo and extracted with dichloromethane and water. The organic phase was washed with sodium bicarbonate solution, brine and ammonium chloride solution, dried over sodium sulfate and concentrated again in vacuo. The protected product precipitated already partly during washing with ammonium chloride and was removed prior to drying with sodium sulfate.

Both, the residue and the precipitate were dissolved in dichloromethane (150 mL) and treated with trifluoroacetic acid (75 mL), followed by stirring overnight at room temperature.

Again, the product already partly precipitated and was removed. The remaining solution was concentrated in vacuo and extracted with dichloromethane and water. The organic phase was washed with bicarbonate solution and brine, dried over sodium sulfate and was finally concentrated in vacuo. During the aqueous workup, the product partly precipitated again. The combined precipitate fractions plus the concentrated fraction from the organic phase were combined and purified by crystallization from refluxing ethyl acetate to give the title compound (12.3 g, 26.8 mmol, 55% yield over 2 steps, 98% purity).

LC-MS (Method B): Rt=1.06 min; MS (ESIpos): m/z=459 [M+H]⁺

¹H-NMR (400 MHz, DMSO-d₆) δ [ppm] 3.92 (s, 2H), 7.30-7.37 (m, 2H), 7.42-7.48 (m, 4H), 7.60 (d, 1H), 7.98 (dd, 1H), 8.18 (s, 1H), 8.39 (d, 1H), 8.74 (s, 1H), 10.83 (s, 1H).

### Example 20

### 2-(2-Fluorophenyl)-N-{3-sulfamoyl-4-[4-(trifluoromethyl)-1H-pyrazol-1-yl]phenyl}-acetamide

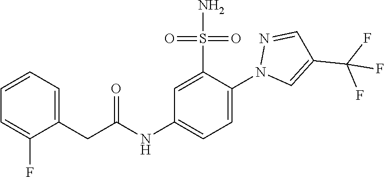 [SMILES: NS(=O)(=O)c1cc(NC(=O)Cc2ccccc2F)ccc1-n1cc(C(F)(F)F)cn1]

5-Amino-N-(2,4-dimethoxybenzyl)-2-[4-(trifluoromethyl)-1H-pyrazol-1-yl]benzenesulfonamide (350 mg, 0.767 mmol) was dissolved in DMF (15 mL) followed by the addition of (2-fluorophenyl)acetic acid (130 mg, 0.843 mmol), N,N-diisopropylethylamine (496 mg, 3.83 mmol) and HATU (466 mg, 1.23 mmol). The reaction mixture was stirred overnight at room temperature. It was then concentrated in vacuo and extracted with dichloromethane and water. The organic phase was washed with brine, dried over sodium sulfate and concentrated again in vacuo.

The residue was dissolved in dichloromethane (10 mL) and treated with trifluoroacetic acid (4.37 g, 38.3 mmol), followed by stirring overnight at room temperature. It was concentrated in vacuo and purified by preparative HPLC (Chromatorex C-18 10 μm, 125×30 mm, acetonitrile/water+0.1% formic acid) %)) to give the title compound (60.5 mg, 0.137 mmol, 18% yield over 2 steps, 98% purity).

LC-MS (Method A): Rt=1.10 min; MS (ESIpos): m/z=443 [M+H]⁺

¹H-NMR (400 MHz, DMSO-d₆) δ [ppm] 3.82 (s, 2H), 7.17-7.23 (m, 2H), 7.31-7.49 (m, 4H), 7.60 (d, 1H), 7.98 (dd, 1H), 8.18 (s, 1H), 8.39 (d, 1H), 8.74 (s, 1H), 10.82 (s, 1H).

### Example 24

### 2-(2-Chlorophenyl)-N-[4-(3-chloro-1H-1,2,4-triazol-1-yl)-3-sulfamoylphenyl]-acetamide

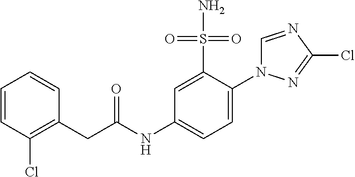 [SMILES: NS(=O)(=O)c1cc(NC(=O)Cc2ccccc2Cl)ccc1-n1cnc(Cl)n1]

According to general procedures GP1.2, GP2.1, GP3.4 and GP4.2, 2-chloro-N-(2,4-dimethoxybenzyl)-5-nitrobenzenesulfonamide (500 mg, 1.29 mmol), 3-chloro-1H-1,2,4-triazole (201 mg, 1.94 mmol) and (2-chlorophenyl)acetic acid (203 mg, 1.19 mmol) were converted without purification of intermediates to the title compound and were purified at the end by preparative HPLC (Waters XBridge C18 5p 100×30 mm, acetonitrile/water+0.2% aqueous ammonia (32%)) (9 mg, 0.0211 mmol, 2% yield over 4 steps, 97% purity).

LC-MS (Method B): Rt=0.70 min; MS (ESIpos): m/z=426 [M+H]⁺

¹H-NMR (600 MHz, DMSO-d₆) δ [ppm]: 3.92 (s, 2H), 7.31-7.35 (m, 2H), 7.43-7.49 (m, 2H), 7.60 (s, 2H), 7.62 (d, 1H), 7.95 (dd, 1H), 8.42 (d, 1H), 8.81 (s, 1H), 10.87 (s, 1H).

### Example 25

### 2-(2-Chlorophenyl)-N-[4-(4-chloro-1H-pyrazol-1-yl)-3-sulfamoylphenyl]acetamide

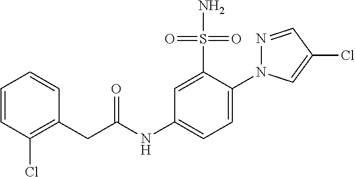 [SMILES: NS(=O)(=O)c1cc(NC(=O)Cc2ccccc2Cl)ccc1-n1cc(Cl)cn1]

According to general procedures GP1.2, GP2.1, GP3.4 and GP4.2, 2-chloro-N-(2,4-dimethoxybenzyl)-5-nitrobenzenesulfonamide (500 mg, 1.29 mmol), 4-chloro-1H-pyrazole (199 mg, 1.94 mmol) and (2-chlorophenyl)acetic acid (313 mg, 1.83 mmol) were converted without purification of intermediates to the title compound and were purified at the end by preparative HPLC (Waters XBridge C18 5p 100×30 mm, acetonitrile/water+0.2% aqueous ammonia (32%)) (55 mg, 0.129 mmol, 10% yield over 4 steps, 99% purity).

LC-MS (Method B): Rt=0.95 min; MS (ESIpos): m/z=425 [M+H]⁺

¹H-NMR (500 MHz, DMSO-d₆) δ [ppm]: 3.91 (s, 2H), 7.31-7.37 (m, 2H), 7.41 (s, 2H), 7.44-7.49 (m, 2H), 7.55 (d, 1H), 7.87 (s, 1H), 7.97 (dd, 1H), 8.35 (d, 1H), 8.38 (d, 1H), 10.81 (s, 1H).

### Example 26

### 2-(2-Chlorophenyl)-N-[4-(4-fluoro-1H-pyrazol-1-yl)-3-sulfamoylphenyl]acetamide

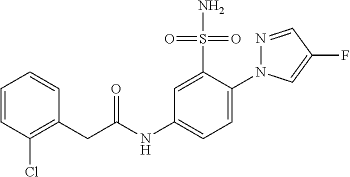 [SMILES: NS(=O)(=O)c1cc(NC(=O)Cc2ccccc2Cl)ccc1-n1cc(F)cn1]

According to general procedures GP1.2, GP2.1, GP3.4 and GP4.2, 2-chloro-N-(2,4-dimethoxybenzyl)-5-nitrobenzenesulfonamide (500 mg, 1.29 mmol), 4-fluoro-1H-pyrazole (167 mg, 1.94 mmol) and (2-chlorophenyl)acetic acid (151 mg, 1.89 mmol) were converted without purification of intermediates to the title compound and were purified at the end by preparative HPLC (Waters XBridge C18 5p 100×30 mm, acetonitrile/water+0.2% aqueous ammonia (32%)) (43 mg, 0.105 mmol, 8% yield over 4 steps, 97% purity).

LC-MS (Method B): Rt=0.88 min; MS (ESIpos): m/z=409 [M+H]⁺

¹H-NMR (500 MHz, DMSO-d₆) δ [ppm]: 3.91 (s, 2H), 7.30-7.37 (m, 2H), 7.39 (s, 2H), 7.43-7.50 (m, 2H), 7.53 (d, 1H), 7.84 (d, 1H), 7.97 (dd, 1H), 8.26 (d, 1H), 8.37 (d, 1H), 10.79 (s, 1H).

### Example 28

### N-[4-(4-Bromo-1H-pyrazol-1-yl)-3-sulfamoylphenyl]-2-(2-chlorophenyl)acetamide

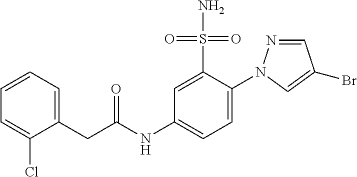 [SMILES: NS(=O)(=O)c1cc(NC(=O)Cc2ccccc2Cl)ccc1-n1cc(Br)cn1]

According to general procedures GP1.2, GP2.2, GP3.5 and GP4.1, 2-chloro-N-(2,4-dimethoxybenzyl)-5-nitrobenzenesulfonamide (400 mg, 1.03 mmol), 4-bromo-1H-pyrazole (228 mg, 1.55 mmol) and (2-chlorophenyl)acetic acid (264 mg, 1.55 mmol) were converted without purification of intermediates to the title compound and were purified at the end by preparative HPLC (Waters XBridge C18 5p 100×30 mm, acetonitrile/water+0.1% formic acid) (27 mg, 0.0575 mmol, 6% yield over 4 steps, 95% purity).

LC-MS (Method A): Rt=1.10 min; MS (ESIpos): m/z=469/471 [M+H]⁺

¹H-NMR (400 MHz, DMSO-d₆) δ [ppm]: 3.90 (s, 2H), 7.29-7.36 (m, 2H), 7.41 (s, 2H), 7.42-7.48 (m, 2H), 7.54 (d, 1H), 7.87 (d, 1H), 7.96 (dd, 1H), 8.34 (d, 1H), 8.37 (d, 1H), 10.80 (s, 1H).

### Example 39

### 2-(2-Chlorophenyl)-N-[4-(4-cyano-1H-pyrazol-1-yl)-3-sulfamoylphenyl]acetamide

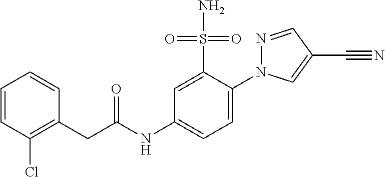 [SMILES: N#Cc1cnn(-c2ccc(NC(=O)Cc3ccccc3Cl)cc2S(N)(=O)=O)c1]

Method 1: Pd/C (10% loading, 350 mg) was added to a solution of 2-(4-cyano-1H-pyrazol-1-yl)-N-(2,4-dimethoxybenzyl)-5-nitrobenzenesulfonamide (9.09 g, 20.5 mmol) in a mixture of methanol (120 mL) and tetrahydrofuran (250 mL) and stirred at room temperature for 3 h under a flow of hydrogen. The catalyst was removed by filtration, followed by washing with tetrahydrofuran and concentration of the filtrate in vacuo. It was extracted with ethyl acetate/water. Sodium carbonate solution was added and it was stirred overnight. The resulting precipitate was removed by filtration and discarded. The organic phase was separated, dried over sodium sulfate and concentrated in vacuo to give crude 5-amino-2-(4-cyano-1H-pyrazol-1-yl)-N-(2,4-dimethoxybenzyl)benzenesulfonamide (6.37 g) that was used without further purification in the next step.

LC-MS (Method B): Rt=1.06 min; MS (ESIpos): m/z=414 [M+H]⁺

¹H-NMR (400 MHz, DMSO-d₆) δ [ppm]: 3.69 (s, 3H), 3.72 (s, 3H), 3.92 (br d, 2H), 6.04 (s, 2H), 6.40-6.48 (m, 2H), 6.78 (dd, 1H), 7.08-7.14 (m, 2H), 7.19 (d, 1H), 7.27 (br t, 1H), 8.25 (s, 1H), 8.70 (s, 1H).

The crude material from the previous step (6.37 g) was dissolved in DMF (87 mL) followed by the addition of (2-chlorophenyl)acetic acid (3.94 g, 23.1 mmol), N,N-diisopropylethylamine (5.97 g, 46.2 mmol) and HATU (8.78 g, 23.1 mmol). The reaction mixture was stirred over the weekend at room temperature. It was then concentrated in vacuo and extracted with ethyl acetate and water. The organic phase was washed with ammonium chloride, sodium bicarbonate solution and brine, dried over sodium sulfate and concentrated again in vacuo to yield crude 2-(2-chlorophenyl)-N-{4-(4-cyano-1H-pyrazol-1-yl)-3-[(2,4-dimethoxybenzyl)-sulfamoyl]phenyl}acetamide (9.77 g) that was used without further purification in the next step.

LC-MS (Method B): Rt=1.27 min; MS (ESIpos): m/z=566 [M+H]⁺

The crude material from the previous step (9.77 g) was dissolved in a mixture of dichloromethane (30 mL) and trifluoroacetic acid (15 mL) and was stirred at room temperature overnight. It was concentrated in vacuo, dissolved in dichloromethane and concentrated in vacuo again to remove remaining trifluoroacetic acid. It was then stirred in a mixture of dichloromethane/water over the weekend. The resulting precipitate was removed by filtration and provided pure title compound (5.40 g, 13.0 mmol, 63% yield over 3 steps, 97% purity). Purity could be further improved by recrystallization form ethyl acetate/hexanes.

LC-MS (Method B): Rt=0.84 min, MS (ESIpos): m/z=416 [M+H]⁺

¹H-NMR (400 MHz, DMSO-d₆) δ [ppm]: 3.91 (s, 2H), 7.29-7.36 (m, 2H), 7.42-7.49 (m, 4H), 7.58 (d, 1H), 7.97 (dd, 1H), 8.31 (d, 1H), 8.39 (d, 1H), 8.86 (d, 1H), 10.84 (br s, 1H).

Method 2: 5-Amino-2-(4-cyano-1H-pyrazol-1-yl)benzenesulfonamide (81 mg, 0.31 mmol) was dissolved in dimethylformamide (1 mL), followed by the addition of N,N-diisopropylethylamine (119 mg, 0.92 mmol), (2-chlorophenyl)acetic acid (63 mg, 0.37 mmol) and HATU (1-[bis(dimethylamino)methylene]-1H-1,2,3-triazolo[4,5-b]pyridinium 3-oxid hexafluorophosphate, 140 mg, 0.37 mmol). The reaction mixture was stirred overnight at room temperature. Then it was concentrated in vacuo, ethyl acetate and water were added and the organic phase was washed with brine, dried over sodium sulfate and was concentrated in vacuo. Purification by preparative HPLC (Chromatorex C-18 10 μm, 125×30 mm, acetonitrile/water+0.1% aqueous ammonia (32%)) gave the title compound (33 mg, 0.0794 mmol, 26% yield, 50% purity).

### Example 170

### N-[4-(4-Chloro-1H-pyrazol-1-yl)-3-sulfamoylphenyl]-2-(4-methoxyphenyl)acetamide

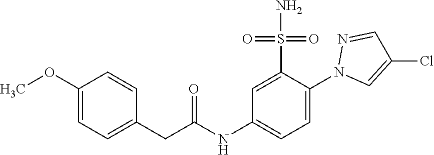 [SMILES: COc1ccc(CC(=O)Nc2ccc(-n3cc(Cl)cn3)c(S(N)(=O)=O)c2)cc1]

According to general procedure GP5.1, 5-amino-2-(4-chloro-1H-pyrazol-1-yl)-N-(2,4-dimethoxybenzyl)benzenesulfonamide (0.20 mmol) and (4-methoxyphenyl)acetic acid (0.40 mmol) were converted to the title compound (12.2 mg, 0.0290 mmol, 14% yield, 100% purity).

LC-MS (Method A): Rt=1.07 min; MS (ESIpos): m/z=421 [M+H]⁺

### Example 321

### N-{6-[1-(Difluoromethyl)-1H-pyrazol-4-yl]-5-sulfamoylpyridin-3-yl}-2-(2-fluorophenyl)acetamide

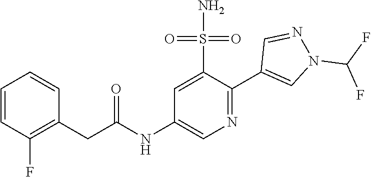 [SMILES: NS(=O)(=O)c1cc(NC(=O)Cc2ccccc2F)cnc1-c1cnn(C(F)F)c1]

5-Amino-2-[1-(difluoromethyl)-1H-pyrazol-4-yl]-N-[(dimethylamino)methylidene]pyridine-3-sulfonamide (400 mg, 1.16 mmol) was dissolved in DMF (10 ml) and (2-fluorophenyl)acetic acid (179 mg, 1.16 mmol) was added followed by the addition of N,N-diisopropylethylamine (1.0 ml, 5.8 mmol) and HATU (530 mg, 1.39 mmol). The reaction was stirred at room temperature for 2 h. The solvent was removed under reduced pressure and ethyl acetate and water were added. The phases were separated and the aqueous phase was washed with ethyl acetate. The combined organic phases were dried over Whatmanfilter and the solvent was removed under reduced pressure. The crude was purified by HPLC (Chromatorex C-18 10 μm, 125×30 mm, acetonitrile/water+0.2% aqueous ammonia (32%)) to yield 30.0 mg (5% yield).

LC-MS (Method B): Rₜ=0.99 min; MS (ESIpos): m/z=481 [M+H]⁺

N-(6-[1-(Difluoromethyl)-1H-pyrazol-4-yl]-5-{[(dimethylamino)methylidene]sulfamoyl}-pyridin-3-yl)-2-(2-fluorophenyl)acetamide (30.0 mg, 62.4 μmol) was dissolved in ammonia in methanol (10 ml, 7 M) and stirred at room temperature. Afterwards the solvent was removed under reduced pressure and the crude was purified by HPLC (Chromatorex C-18 10 μm, 125×30 mm, acetonitrile/water+0.2% aqueous ammonia (32%)) to yield the title compound (10.1 mg, 99% purity, 38% yield).

LC-MS (Method B): Rₜ=0.66 min; MS (ESIpos): m/z=426 [M+H]⁺

¹H-NMR (400 MHz, DMSO-de): δ [ppm]=3.83 (s, 2H), 7.15-7.24 (m, 2H), 7.31-7.38 (m, 1H), 7.42 (td, 1H), 7.71-8.07 (m, 3H), 8.29 (s, 1H), 8.70 (s, 1H), 8.78 (d, 1H), 8.93 (d, 1H), 10.86 (s, 1H).

### Example 326

### 2-(2-Chlorophenyl)-N-{4-[1-(difluoromethyl)-1H-pyrazol-4-yl]-3-sulfamoylphenyl}acetamide

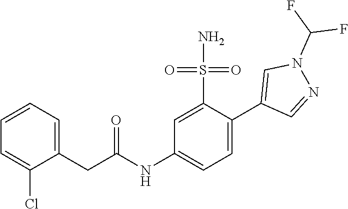 [SMILES: NS(=O)(=O)c1cc(NC(=O)Cc2ccccc2Cl)ccc1-c1cnn(C(F)F)c1]

N-(4-Bromo-3-{[(dimethylamino)methylidene]sulfamoyl}phenyl)-2-(2-chlorophenyl)acetamide (1.50 g, 3.27 mmol), 1-(difluoromethyl)-4-(4,4,5,5-tetramethyl-1,3,2-dioxaborolan-2-yl)-1H-pyrazole (958 mg, 3.92 mmol) and potassium fluoride (418 mg, 7.19 mmol) were dissolved in DMF (36 ml). The mixture was purged with argon for 5 minutes, followed by addition of bis(tri-tert-butylphosphine)palladium(0) (CAS 53199-31-8) (83.5 mg, 163 μmol). The reaction was heated for 1 h at 100° C., filtered over a glass fibre filter and the procedure was repeated. Afterwards, the solvent was removed under reduced pressure and ethyl acetate and water were added. The phases were separated and the aqueous phase was washed with ethyl acetate. The combined organic phases were dried over Whatmanfilter and the solvent was removed under reduced pressure. The crude was used in the next step without further purification (2.78 g).

2-(2-Chlorophenyl)-N-(4-[1-(difluoromethyl)-1H-pyrazol-4-yl]-3-{[(dimethylamino)-methylidene]sulfamoyl}phenyl)acetamide (2.78 g, 5.61 mmol) was dissolved in methanol (90 ml) and treated with 25% aqueous ammonia solution (90 ml) at room temperature until completion of the reaction. The solvent was removed under reduced pressure and the crude was purified by chromatography on silica gel (Biotage, 8% ethanol in dichloromethane) and subsequently by HPLC (Chromatorex C-18 10 μm, 125×30 mm, acetonitrile/water+0.2% aqueous ammonia (32%)) to yield the title compound (1.09 g, 99% purity, 34% over 2 steps).

LC-MS (Method B): Rt=0.94 min; MS (ESIpos): m/z=441 [M+H]⁺

¹H-NMR (400 MHz, DMSO-d): δ [ppm]=3.89 (s, 2H), 7.31-7.35 (m, 2H), 7.41 (s, 2H), 7.43-7.50 (m, 3H), 7.69-8.00 (m, 2H), 8.02 (m, 1H), 8.36 (d, 1H), 8.43 (m, 1H), 10.65 (s, 1H).

### Example 331

### 2-(2-Chlorophenyl)-N-{3-sulfamoyl-4-[5-(trifluoromethyl)pyridin-3-yl]phenyl}-acetamide

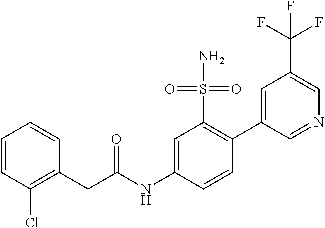 [SMILES: NS(=O)(=O)c1cc(NC(=O)Cc2ccccc2Cl)ccc1-c1cncc(C(F)(F)F)c1]

N-(4-Bromo-3-{[(dimethylamino)methylidene]sulfamoyl}phenyl)-2-(2-chlorophenyl)acetamide (500 mg, 1.09 mmol) and [5-(trifluoromethyl)pyridin-3-yl]boronic acid (520 mg, 2.72 mmol) were dissolved in n-propanol (15 ml) and bis(triphenylphosphine)palladium(II) dichloride (CAS 13965-03-2) (38.4 mg, 54.5 μmol), triphenylphosphine (14.3 mg, 54.5 μmol), potassium fluoride (23.1 mg, 270 μmol) and aq. potassium carbonate solution (1.4 ml, 2.0 M, 2.7 mmol) were added. The reaction was heated at 100° C. for 1 h in the microwave (1 bar/15 W). Afterwards the mixture was filtered over Celite, the solvent was removed under reduced pressure and the crude was co-distilled with THE and used without further purification in the next step.

2-(2-Chlorophenyl)-N-(3-{[(dimethylamino)methylidene]sulfamoyl}-4-[5-(trifluoromethyl)-pyridin-3-yl]phenyl)acetamide (1.50 g, 2.86 mmol) was dissolved in methanol (29 ml) and treated with 32% aqueous sodium hydroxide (1.6 ml) at 80° C. until completion of the reaction. The solvent was removed under reduced pressure, the crude was dissolved in dichloromethane and washed with water. The phases were separated and the combined organic phases were dried over Whatmanfilter and the solvent was removed under reduced pressure. The crude was purified by chromatography on silica gel (Biotage, 40% ethyl acetate in hexane) and subsequently by HPLC (Chromatorex C-18 10 μm, 125×30 mm, acetonitrile/water+0.1% formic acid) to yield the title compound (562 mg, 95% purity, 40% yield over 2 steps).

LC-MS (Method A): Rt=1.13 min; MS (ESIneg): m/z=468 [M−H]⁻

¹H-NMR (400 MHz, DMSO-d): δ [ppm]=3.91 (s, 2H), 7.28-7.37 (m, 2H), 7.39 (d, 1H), 7.42-7.51 (m, 4H), 7.88 (dd, 1H), 8.10-8.16 (m, 1H), 8.40 (d, 1H), 8.81 (d, 1H), 8.96 (d, 1H), 10.73 (s, 1H).

### Example 349

### 2-(2-Chlorophenyl)-N-[4-(1-cyclopropyl-1H-pyrazol-4-yl)-3-sulfamoylphenyl]acetamide

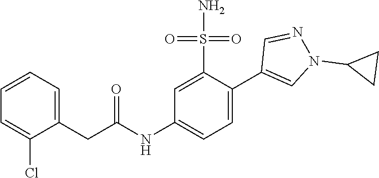 [SMILES: NS(=O)(=O)c1cc(NC(=O)Cc2ccccc2Cl)ccc1-c1cnn(C2CC2)c1]

N-(4-Bromo-3-{[(dimethylamino)methylidene]sulfamoyl}phenyl)-2-(2-chlorophenyl)acetamide (500 mg, 1.09 mmol), 1-cyclopropyl-4-(4,4,5,5-tetramethyl-1,3,2-dioxaborolan-2-yl)-1H-pyrazole (510 mg, 2.18 mmol) and potassium fluoride (139 mg, 2.4 mmol) were dissolved in dry and degased DMF (30 ml) and the solution was purged again with argon for 5 minutes followed by addition of bis(tri-tert-butylphosphine)palladium(0) (CAS 53199-31-8) (28 mg, 54 μmol). The reaction was heated for 2 h at 100° C. Afterwards the mixture was filtered over Celite, the solvent was removed under reduced pressure and the crude was used without further purification in the next step.

2-(2-Chlorophenyl)-N-[4-(1-cyclopropyl-1H-pyrazol-4-yl)-3-{[(dimethylamino)methylidene]-sulfamoyl}phenyl]acetamide (560 mg, 1.15 mmol) was dissolved in methanol (54 ml) and treated with 32% aqueous sodium hydroxide (560 μl) at 80° C. until completion of the reaction. The solvent was removed under reduced pressure and purified by chromatography on silica gel (Biotage, ethyl acetate/hexane) and subsequently by HPLC (Waters XBrigde C18 5p 100×30 mm, acetonitrile/water+0.2% aqueous ammonia (32%)) to yield the title compound (192 mg, 95% purity, 37% yield over 2 steps).

LC-MS (Method B): Rt=0.96 min; MS (ESIneg): m/z=429 [M−H]⁻

¹H-NMR (400 MHz, DMSO-de): δ [ppm]=0.94-1.01 (m, 2H), 1.05-1.10 (m, 2H), 3.67-3.80 (m, 1H), 3.88 (s, 2H), 7.19 (s, 2H), 7.30-7.35 (m, 2H), 7.40-7.48 (m, 3H), 7.67 (d, 1H), 7.81 (dd, 1H), 8.04 (s, 1H), 8.31 (d, 1H), 10.57 (s, 1H).

### Example 360

### 2-(2-Chlorophenyl)-N-[4-(1-methyl-1H-pyrazol-4-yl)-3-sulfamoylphenyl]acetamide

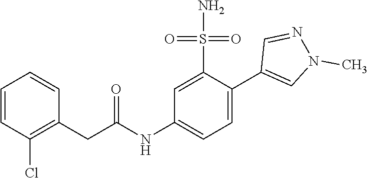 [SMILES: Cn1cc(-c2ccc(NC(=O)Cc3ccccc3Cl)cc2S(N)(=O)=O)cn1]

N-(4-Bromo-3-{[(dimethylamino)methylidene]sulfamoyl}phenyl)-2-(2-chlorophenyl)acetamide (900 mg, 1.96 mmol) and 1-methyl-4-(4,4,5,5-tetramethyl-1,3,2-dioxaborolan-2-yl)-1H-pyrazole (490 mg, 2.35 mmol) were dissolved in DMF (25 ml) followed by addition of potassium fluoride (251 mg, 4.32 mmol). The solution was purged with argon for 5 minutes and bis(tri-tert-butylphosphine)palladium(0) (CAS 53199-31-8) (50.1 mg, 98.1 μmol) was added. The reaction was heated for 1 h at 100° C. The mixture was filtered via a glasfiber filter and the solvent was removed under reduced pressure. The crude was subjected once more to the reaction procedure described above. Afterwards, the solvent was removed under reduced pressure and ethyl acetate and water were added. The phases were separated and the aqueous phase was extracted with ethyl acetate. The combined organic phases were dried over Whatmanfilter and the solvent was removed under reduced pressure. The crude was used in the next step without further purification (2.39 g).

2-(2-Chlorophenyl)-N-[3-{[(dimethylamino)methylidene]sulfamoyl}-4-(1-methyl-1H-pyrazol-4-yl)phenyl]acetamide (2.39 g, 5.20 mmol) was dissolved in methanol (80 ml) and treated with 25% aqueous ammonia solution (80 ml) at room temperature. UPLC indicated incomplete reaction, 25% aqueous ammonia solution (80 ml) was added and stirring was continued until completion of the reaction. The solvent was removed under reduced pressure and the crude was purified by chromatography on silica gel (Biotage, 10% ethanol in dichloromethane) followed by HPLC (Waters XBrigde C18 5p 100×30 mm, acetonitrile/water+0.2% aqueous ammonia (32%)) to yield the title compound (327 mg, 98% purity, 15% yield over 2 steps).

LC-MS (Method B): Rt=0.86 min; MS (ESIneg): m/z=403 [M−H]⁻

¹H-NMR (400 MHz, DMSO-de): δ [ppm]=3.78-3.96 (m, 5H), 7.17 (s, 2H), 7.28-7.37 (m, 2H), 7.38-7.52 (m, 3H), 7.66 (d, 1H), 7.82 (dd, 1H), 7.96 (s, 1H), 8.32 (d, 1H), 10.57 (s, 1H).

### Example 380

### 2-(2-Chlorophenyl)-N-{5-sulfamoyl-6-[4-(trifluoromethyl)-1H-pyrazol-1-yl]pyridin-3-yl}acetamide

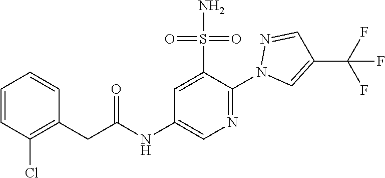 [SMILES: NS(=O)(=O)c1cc(NC(=O)Cc2ccccc2Cl)cnc1-n1cc(C(F)(F)F)cn1]

5-Amino-N-[(dimethylamino)methylidene]-2-[4-(trifluoromethyl)-1H-pyrazol-1-yl]pyridine-3-sulfonamide (250 mg, 690 μmol) and (2-chlorophenyl)acetic acid (177 mg, 1.03 mmol) were dissolved in DMF (10 ml) and N,N-diisopropylethylamine (600 μl, 3.4 mmol) and 2,4,6-tripropyl-1,3,5,2,4,6-trioxatriphosphinane 2,4,6-trioxide (310 μl, 1.0 mmol) were added successively. The reaction was stirred at room temperature over night. Afterwards, the solvent was removed under reduced pressure and ethyl acetate and water were added. The phases were separated and the organic phase was dried over Whatmanfilter. The solvent was removed under reduced pressure and the crude was used without further purification in the next step (400 mg).

2-(2-Chlorophenyl)-N-(5-{[(dimethylamino)methylidene]sulfamoyl}-6-[4-(trifluoromethyl)-1H-pyrazol-1-yl]pyridin-3-yl)acetamide (400 mg, 777 μmol) was dissolved in methanol (37 ml) and treated with 40% aqueous sodium hydroxide solution (24 μl, 1.9 mmol) for 1 h at 50° C. Afterwards, the solvent was removed under reduced pressure and the crude was purified by HPLC chromatography (Chromatorex C-18 10 μm, 125×30 mm, acetonitrile/water+0.2% aqueous ammonia (32%)) to yield 3.8 mg of the title compound (95% purity, 1% yield over 2 steps).

LC-MS (Method B): Rt=0.93 min; MS (ESIpos): m/z=460 [M+H]⁺

¹H-NMR (400 MHz, DMSO-d): [ppm]=3.95 (s, 2H), 7.29-7.39 (m, 2H), 7.43-7.52 (m, 2H), 7.61 (s, 2H), 8.24 (s, 1H), 8.86 (d, 1H), 8.91 (d, 1H), 8.97 (d, 1H), 11.06 (s, 1H).

### Example 381

### 2-(2-Fluorophenyl)-N-{5-sulfamoyl-6-[4-(trifluoromethyl)-1H-pyrazol-1-yl]pyridin-3-yl}acetamide

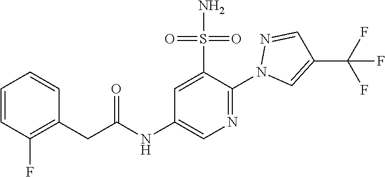 [SMILES: NS(=O)(=O)c1cc(NC(=O)Cc2ccccc2F)cnc1-n1cc(C(F)(F)F)cn1]

The title compound was obtained analoguous to Example 380 starting from 5-amino-N-[(dimethylamino)methylidene]-2-[4-(trifluoromethyl)-1H-pyrazol-1-yl]pyridine-3-sulfonamide (250 mg, 690 μmol) in 2 steps after HPLC purification (Chromatorex C-18 10 μm, 125×30 mm, acetonitrile/water+0.2% aqueous ammonia (32%)) (12.8 mg, 90% purity, 4% yield).

LC-MS (Method B): Rt=0.90 min; MS (ESIpos): m/z=444 [M+H]⁺ 1H-NMR (400 MHz, DMSO-d6): δ [ppm]=3.85 (s, 2H), 7.13-7.26 (m, 2H), 7.29-7.47 (m, 2H), 7.61 (s, 2H), 8.23 (s, 1H), 8.86 (d, 1H), 8.91 (d, 1H), 8.97 (s, 1H), 11.04 (s, 1H).

### Example 387

### 2-(2-Chlorophenyl)-N-[6-(4-chloro-1H-pyrazol-1-yl)-5-sulfamoylpyridin-3-yl]acetamide

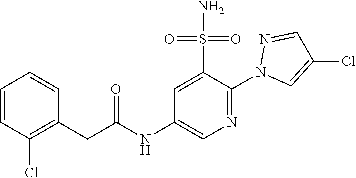 [SMILES: NS(=O)(=O)c1cc(NC(=O)Cc2ccccc2Cl)cnc1-n1cc(Cl)cn1]

According to general procedure GP6.1, crude 5-amino-2-(4-chloro-1H-pyrazol-1-yl)-N-[(dimethylamino)methylene]pyridine-3-sulfonamide (100 mg) and (2-chlorophenyl)acetic acid (77.8 mg, 0.46 mmol) were converted without purification of intermediates to 2 the title compound. The title compound precipitated during the reaction and was obtained by filtration, no further purification was necessary (38 mg, 0.0891 mmol, 27% yield over 5 steps, 99% purity).

LC-MS (Method A): Rt=1.13 min, MS (ESIpos): m/z=426 [M+H]⁺

¹H-NMR (400 MHz, DMSO-d₆) δ [ppm]: 3.94 (s, 2H), 7.30-7.37 (m, 2H), 7.43-7.49 (m, 2H), 7.60 (s, 2H), 7.94 (d, 1H), 8.58 (d, 1H), 8.84 (d, 1H), 8.89 (d, 1H), 11.01 (s, 1H).

Biological Assays

The following assays can be used to illustrate the commercial utility of the compounds according to the present invention.

Examples were tested in selected biological assays one or more times. When tested more than once, data are reported as either average (avg) values or as median values, wherein
the average value, also referred to as the arithmetic mean value, represents the sum of the obtained values divided by the number of values obtained, and
the median value represents the middle number of the group of obtained values when ranked in ascending or descending order. If the number of values in the data set is odd, the median is the middle value. If the number of values in the data set is even, the median is the arithmetic mean of the two middle values.

When no meaningful calculation of average values or median values is possible due to the existence of measurement values falling outside the detection range of the assay (indicated by < or > in the tables below) all individual measurement values are indicated.

Examples were synthesized one or more times. When synthesized more than once, data from biological assays represent average values or median values calculated utilizing data sets obtained from testing of one or more synthetic batch.

In Vitro Studies

Human P2X4 HEK Cell FLIPR Assay

HEK293 cells stably expressing human P2X4 were plated in poly-D-lysine-coated 384-well plates at a seeding density of 30000 cells/well and incubated overnight. P2X4 function was assessed by measuring intracellular calcium changes using the calcium-chelating dye Fluo8-AM (Molecular Devices) on a fluorescent imaging plate reader (FLEX/FLIPR station; Molecular Devices). On the day of the assay, the medium was removed and the cells were incubated for 30 min at 37° C. and 5% CO₂ in 30 μL of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4). Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) were added in a volume of 10 μL and allowed to incubate for 30 min at room temperature. The final assay DMSO concentration was 0.5%. The agonist, Bz-ATP (Tocris), was added in a volume of 10 μL at a concentration representing the EC₈₀ value. The EC₈₀ value of Bz-ATP was determined each assay day prior to compound profiling. The fluorescence was measured for an interval of 120 sec at 2 sec intervals. The excitation and emission wavelengths used to monitor fluorescence were 470-495 nm and 515-575 nm, respectively. The data was analyzed based on the increase in peak relative fluorescence units (RFU) compared to the basal fluorescence and the data was normalized to the agonist control. The compounds were tested in triplicates per plate and mean values were plotted in Excel XL-Fit to determine IC₅₀ values, percentage of maximal inhibition and the Hill coefficients.

| | Human P2X4 HEK | Human P2X4 HEK |
|---|---|---|
| Example | Cells (FLIPR Assay) | Cells (FLIPR Assay) |
| Number | avg IC₅₀ [nM] | avg Efficacy [%] |
| 19 | 47 | 71 |
| 20 | 140 | 67 |
| 24 | 45 | 92 |
| 25 | 26 | 93 |
| 26 | 27 | 85 |
| 28 | 31 | 90 |
| 39 | 32 | 96 |
| 170 | 126 | 70 |
| 321 | 87 | 84 |
| 326 | 17 | 95 |
| 331 | 25 | 66 |
| 349 | 37 | 99 |
| 360 | 132 | 93 |
| 380 | 299 | 69 |
| 381 | 1241 | 77 |
| 387 | 34 | 73 |

FLIPR Methods for h/m/rP2X4 1321 N1 Astrocytoma Cells

1321N1 Astrocytoma cells stably expressing human P2X4 or rat P2X4 or mouse P2X4 were plated in Collagen ITO-treated microplate at a seeding density of 10000 cells/well and incubated overnight. P2X4 function was assessed by measuring intracellular calcium changes using the calcium-chelating dye Fluo8-AM (Molecular Devices) on a fluorescent imaging plate reader (FLEX/FLIPR station; Molecular Devices). On the day of the assay, the medium was removed and the cells were incubated for 30 min at 370 C and 5% CO₂ in 30 μL of dye buffer (Hank's balanced salt solution, 10 mM HEPES, 1.8 mM CaCl₂, 1 mM MgCl₂, 2 mM probenecid, 5 mM D-glucose monohydrate, 5 μM Fluo8-AM, pH=7.4). Compounds diluted in probenecid buffer (Hank's balanced salt solution, 10 mM HEPES, 1.8 mM CaCl₂, 1 mM MgCl₂, 2 mM probenecid, 5 mM D-glucose monohydrate, pH=7.4) were added in a volume of 10 μL and allowed to incubate for 30 min at room temperature. The final assay DMSO concentration was 0.25%. The agonist, Mg-ATP (Sigma), was added in a volume of 10 μL at a concentration representing the EC₈₀ value. EC₈₀ was determined to be 0.5 μM for human and mouse P2X4 and 5 μM for rat P2X4. The fluorescence was measured for an interval of 120 sec at 2 sec intervals. The excitation and emission wavelengths used to monitor fluorescence were 470-495 nm and 515-575 nm, respectively. The data was analyzed based on the increase in peak relative fluorescence units (RFU) compared to the basal fluorescence and the data was normalized to the agonist control. The compounds were tested in triplicates per plate and mean values were plotted in Excel XLFit to determine IC50 values, percentage of maximal inhibition and the Hill coefficients.

| | Human P2X4 1321N1 | Human P2X4 1321N1 |
|---|---|---|
| | Astrocytoma Cells | Astrocytoma Cells |
| Example | (FLIPR Assay) | (FLIPR Assay) |
| Number | avg IC₅₀ [nM] | avg Efficacy [%] |
| 19 | 57 nM | 60% |
| 39 | 64 nM | 83% |
| 326 | 46 nM | 80% |
| | Mouse P2X4 1321N1 | Mouse P2X4 1321N1 |
| | Astrocytoma Cells | Astrocytoma Cells |
| Example | (FLIPR Assay) | (FLIPR Assay) |
| Number | avg IC₅₀ [nM] | avg Efficacy [%] |
| 19 | 43 nM | 69% |
| 39 | 37 nM | 87% |
| 326 | 26 nM | 87% |
| | Rat P2X4 1321N1 | Rat P2X4 1321N1 |
| | Astrocytoma Cells | Astrocytoma Cells |
| Example | (FLIPR Assay) | (FLIPR Assay) |
| Number | avg IC₅₀ [nM] | avg Efficacy [%] |
| 19 | 71 nM | 57% |
| 39 | 308 nM | 87% |
| 326 | 325 nM | 99% |

Human P2X4 HEK Cell Elektophysiology Assay

Electrophysiology Assay A

HEK293 cells stably expressing human P2X4 were seeded in T75 cell culturing flasks at a density of 7*106 cells and incubated overnight. P2X4 function was assayed using the automated patch clamp platform PatchLiner (Nanion) in single hole mode. Composition of extracellular buffer was (in mM) NaCl 145, KCl 4, HEPES 10, CaCl₂) 1, MgCl₂ 0.5, D-glucose monohydrate 10, pH=7.4. The intracellular buffer contained (in mM): CsF 135, EGTA 1, HEPES 10, NaCl 10, pH 7.2. On the day of the assay, cells were harvested using Accumax (Sigma) and were resuspended in extracellular buffer. The ligand agonist, adenosine 5′-trisphosphate (ATP, 5 μM) was added in a volume of 5 μL, directly washed off by extracellular buffer (40 μL). The cells were voltage clamped at −80 mV and ligand was applied every 5 min. for 20 min. Over this period the agonist response was stable and compounds were measured in single concentration per well mode. Compounds diluted in extracellular buffer (final assay DMSO concentration 0.3%) were added in a volume of 40 μL and allowed to incubate for 8 min at room temperature. The data was analyzed based on the decrease in peak current amplitude and normalized to the agonist control. Mean values were plotted in Excel XLFit to determine IC₅₀ values, percentage of maximal inhibition and the Hill coefficients.

| | Human P2X4 HEK Cells |
|---|---|
| Example | (PatchLiner Electrophysiology) |
| Number | avg IC₅₀ [nM] |
| 19 | 111 |
| 39 | 138 |
| 326 | 57 |
| 380 | 320 |

Electrophysiology Assay B

Cell culture conditions: HEK-293 mito-Photina pcDNA3(neo-)/pPURO N/pcDNA3_P2RX4, clone 2a/4 (HEK-293 mito-Photina/hP2RX4) cells were cultured in EMEM Minimum Essential Medium Eagle with Earl's salts Balanced Salt Solution (BioWhittaker cat. BE12-125F) supplemented with 5 mL of 200 mM Ultraglutaminel (BioWhittaker cat. BE17-605E/U1), 5 mL of 100× Penicillin/Streptomycin (BioWhittaker cat. DE17-602E; final concentration 1%), 4 mL of 50 mg/mL G418 (Sigma cat. G8168-100 mL; final concentration 400 μg/mL), 10 μL of 10 mg/mL Puromicin (InvivoGen cat. ant-pr-1; final concentration 0.2 μg/mL) and 50 mL of Fetal Bovine Serum (Sigma cat. F7524; final concentration 10%).

Experimentalprotocol: HEK-293 cell lines are seeded 72 or 96 hours before experiment, at a concentration of 5 or 2.5 million cells, respectively onto a T225 flask. Just before the experiments cells are washed twice with D-PBS w/o Ca2+/Mg2+(Euroclone cat. ECB4004L) and detached from the flask with trypsin-EDTA (Sigma, cat. T4174 diluted 1/10). Cells are then re-suspended in the suspension solution: 25 mL EX-CELL ACF CHO medium (Sigma, cat. C5467); 0.625 mL HEPES (BioWhittaker, cat. BE17-737E); 0.25 mL of 100× Penicillin/Streptomycin (BioWhittaker, cat. DE17-602E), 0.1 mL of Soybean Trypsin Inhibitor 10 mg/mL (Sigma, cat. T6522) and placed on the QPatch 16X.

Compound preparation and storage: Compound stock solutions (10 mM; 100% DMSO; stored at −20° C.) were used. Fresh solutions from stock (1 or 3 mM, 100% DMSO) were prepared just before the experiments (0.1% final DMSO concentration).

DMSO solution was obtained from SIGMA (cat. #D-5879) and stored at room temperature.

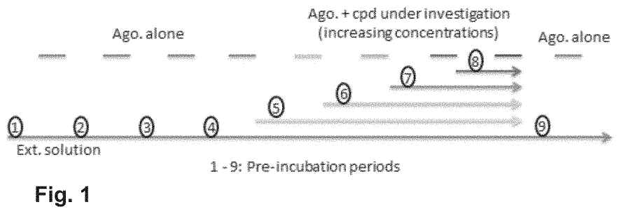

Patch clamp analysis with QPatch16X (FIG. 1): Standard whole-cell voltage clamp experiments are performed at room temperature using the multihole technology.

For the voltage clamp experiments on hP2X4, data are sampled at 2 KHz. After establishment of the seal and the passage in the whole cell configuration, the cells are held at −90 mV and the hP2X4 current is evoked by the agonist in the absence (vehicle period, i.e. 0.1% DMSO) or in the presence of the compound under investigation at increasing concentrations; see the application protocol in FIG. 1.

Output: the maximum inward current induced by the agonist (ATP 5 microM).

The intracellular solution contained (mM) 135 CsF, 10 NaCl, 1 EGTA, 10 HEPES (pH 7.2 with CsOH) whereas the extracellular solution (mM) 145 NaCl, 4 KCl, 0.5 MgCl2, 1 CaCl2, HEPES, 10 Glucose (pH 7.4 with NaOH).

For data collection, the Sophion software was used and the analysis was performed off-line using Excel and GraphPad Prism.

When possible, i.e. when the % of inhibition with the highest concentration tested was more than 50%, the dose-response curves data were fitted with the following equation:

Y=100/(1+10\hat{ }((Log IC50−X)*HillSlope))

[X is log of concentration; Y is normalized response (100% down to 0%, decreasing as X increases); Log IC₅₀ same log units as X; HillSlope is unitless slope factor or hill slope]

| | Human P2X4 HEK Cells | Number |
|---|---|---|
| Example | (QPatch Electrophysiology) | of |
| Number | avg IC₅₀ [nM] | Exp. |
| 19 | 278 | 4 |
| 20 | 195 | 4 |
| 25 | 222 | 5 |
| 26 | 222 | 5 |
| 321 | 227 | 4 |
| 326 | 51 | 6 |
| 331 | 155 | 5 |
| 349 | 117 | 3 |
| 360 | 205 | 3 |
| 380 | 157 | 4 |
| 381 | 2583 | 3 |

Ex Vivo Studies

Human Monocyte P2X4 Assay

The principle of the assay is to measure calcium influx through endogenous P2X4 channels into primary human monocytes, following activation by 2′,3′-O-(4-benzoyl-benzoyl)-ATP (Bz-ATP). Intracellular calcium concentration changes were measured with a Flipr™ (Molecular Devices) device using a calcium sensitive dye (Fluo-8). In primary monocytes P2X4 is located at the lysosome membrane, therefore exocytosis has to be triggered to expose P2X4 at the cellular membrane.

Human peripheral blood mononuclear cells (PBMCs) from anticoagulated blood (blood cells, BC) were isolated via density gradient centrifugation. Whole blood was diluted 1:3 with PBS. Samples of 30 mL were layered carefully on top of 15 mL Biocoll (BIOCHROM) in 50 mL centrifuge tubes (Falcon). Tubes were centrifuged at 914×g for 25 min at RT without brake. The PBMC layer was removed with a 10 mL pipette and transferred into tubes with ice-cold PBS in a total volume of 50 mL. Cells were washed twice by pelleting at 300×g at 4° C., for 10 min and for 5 min respectively. PBMCs were re-suspended in 10 mL medium (X-vivo, Biozym Scientific) and counted in a Neubauer chamber.

Monocytes were isolated by negative selection using the Monocyte isolation kit II from Miltenyi (#130-091-153) according to the instructions. Isolation should be done fast and cells and solutions should be kept on ice at any time. PBMCs in batches of 10exp8 cells were pelleted (300×g, 10 min) and re-suspended with 300 μL MACS buffer in a 50 mL Falcon tube. FcR Blocking reagent (100 μl) and Biotin-Ab (100 μl) were added, mixed and incubated on ice for 10 min. MACS buffer (300 μL) and anti-Biotin Micro-beads (100 μL) were added, mixed and incubated on ice for 15 min. Cells were washed by pelleting (300×g for 10 min) and re-suspended in 500 μL MACS buffer. For each batch one separation column was placed in the MACS separator and rinsed with 3 mL MACS buffer. The cell suspension was added to the column, followed by 3×3 mL MACS buffer for washing, and the eluent containing the monocytes was collected. Cells were pelleted (300×g for 10 min), re-suspended in X-vivo medium and counted. Monocytes were seeded into fibronectin-coated micro-plates (384-well, black, flat transparent bottom; Corning #3848) at a density of 30,000 cells/well in 50 μL, and cultivated over night (37° C., 5% CO₂).

Test substances were dissolved in 100% DMSO at a stock concentration of 10 mM and stored at −20° C. in aliquots. Serial dilutions (2×) were prepared in DMSO and diluted 500× with assay buffer to generate the antagonist plate. In the Flipr measurement, 10 μL per well were transferred (4× dilution) and a final top concentrations of 5 μM and 0.05% DMSO were obtained in the assay. Agonist BzATP was stored at 10 mM in aliquots and diluted to an intermediate concentration of 15 μM to generate the agonist plate. In the Flipr measurement, 10 μL per well were transferred (5× dilution) so that a final assay concentration of 3 μM was obtained.

For the experiment, the medium of the cell plate was discarded manually and 70 μL/well loading buffer was added and incubated for 1 h (37° C., 5% CO₂). Loading buffer contained HBSS (w/o calcium/magnesium), 10 mM Hepes pH 7.4, 5 μM Fluo-8 (AM) (Tebu-bio) and 50 mM methylamine (Sigma) to trigger exocytosis. Loading buffer was discarded manually and 30 μL/well low-calcium assay buffer (5 mM KCl, 145 mM NaCl, 0.5 mM CaCl₂), 13 mM glucose, 10 mM Hepes pH 7.4) was added. The antagonist plate was transferred (10 μL/well) and after 15 min at RT the agonist plate (10 μL/well) was transferred.

Agonist addition was recorded for 240 seconds after a 10 second baseline. For analysis, a baseline correction was applied, and the maximum of the curve was extracted. Data were normalized towards 0% inhibition (signal at 3 μM BzATP) and 100% inhibition (absence of BzATP stimulation) and fitted with a four-parameter sigmoidal inhibition curve using Prism Graph Pad to obtain IC₅₀ values.

| | Human P2X4 Monocytes |
|---|---|
| Example | (FLIPR Assay) |
| Number | IC₅₀ (Efficacy) for different donors |
| 19 | 59 nM (57%), 21 nM (74%), 76 nM (48%), 59 nM (46%), 45 nM (93%) |
| 24 | 141 nM (79%), 34 nM (77%), 91 nM (88%) |
| 25 | 27 nM (87%), 5 nM (82%), 127 nM (70%), 87 nM (70%), 118 nM (70%), |
| | 59 nM (53%), 63 nM (68%), 39 nM (111%) |
| 26 | 290 nM (71%), 182 nM (88%) |
| 28 | 78 nM (64%), 164 nM (90%) |
| 39 | 105 nM (88%), 32 nM (81%), 71 nM (78%) |
| 170 | 303 nM (60%), 183 nM (69%), 110 nM (54%) |
| 321 | 158 nM (49%), 94 nM (49%), 157 nM (60%), 537 nM (60%), 173 nM |
| | (34%), 331 nM (46%), 39 nM (95%) |
| 326 | 407 nM (86%), 167 nM (89%), 149 nM (82%), 45 nM (91%), 49 nM (70%) |
| 380 | 263 nM (70%), 251 nM (70%), 434 nM (70%), 93 nM (43%), 50 nM |
| | (32%), 207 nM (107%) |
| 387 | 122 nM (35%), 104 nM (51%) |

Human Whole Blood P2X4 Assay

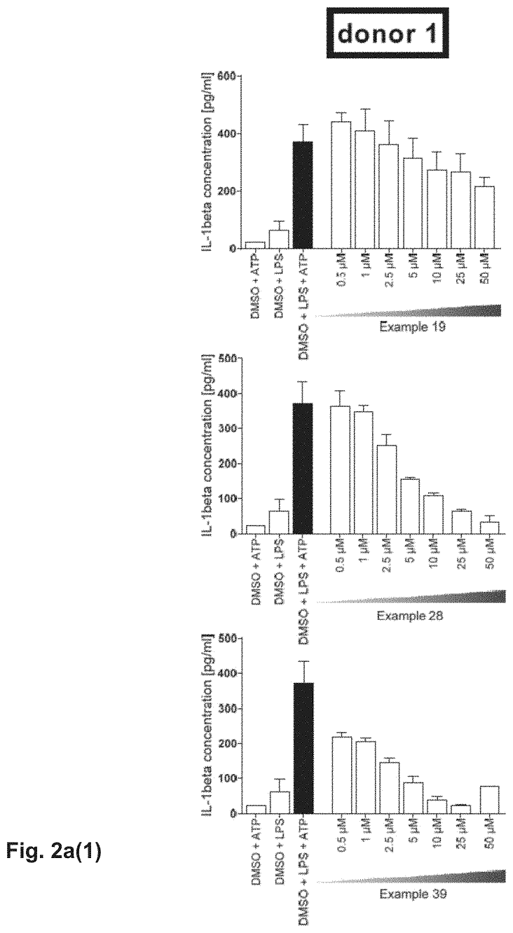

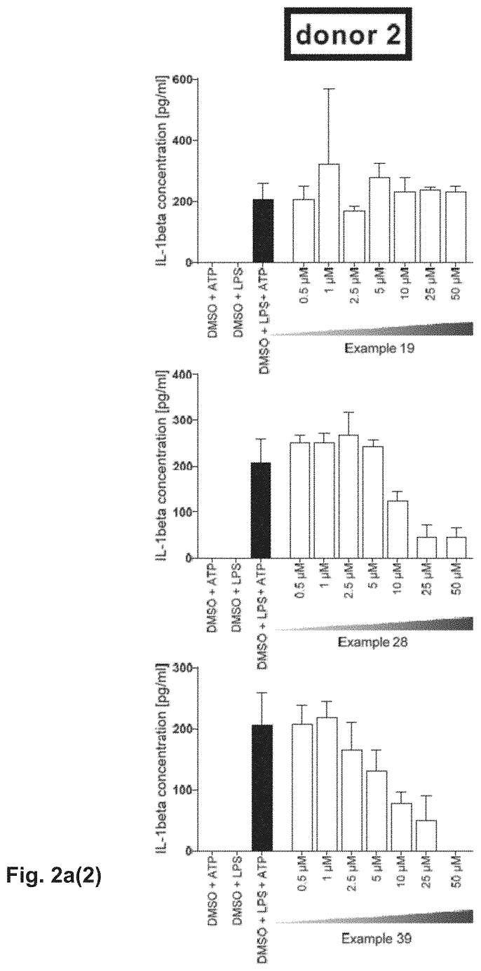

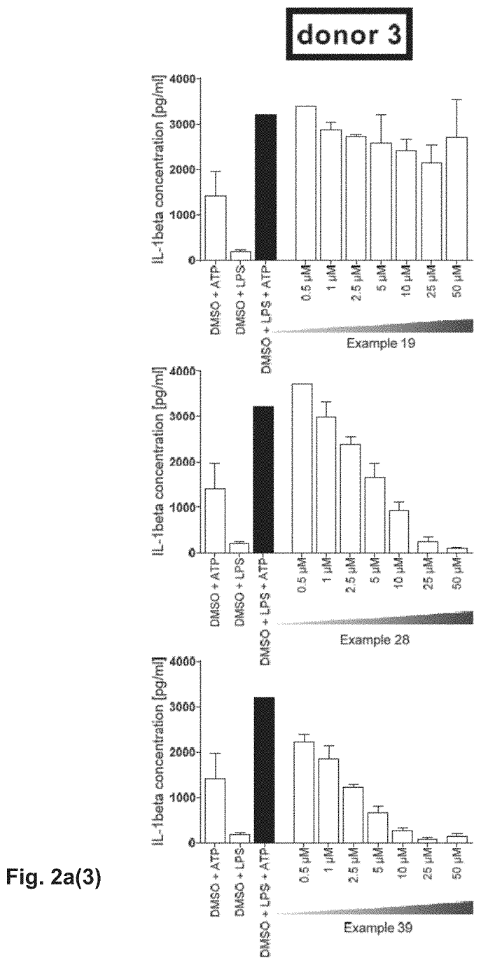

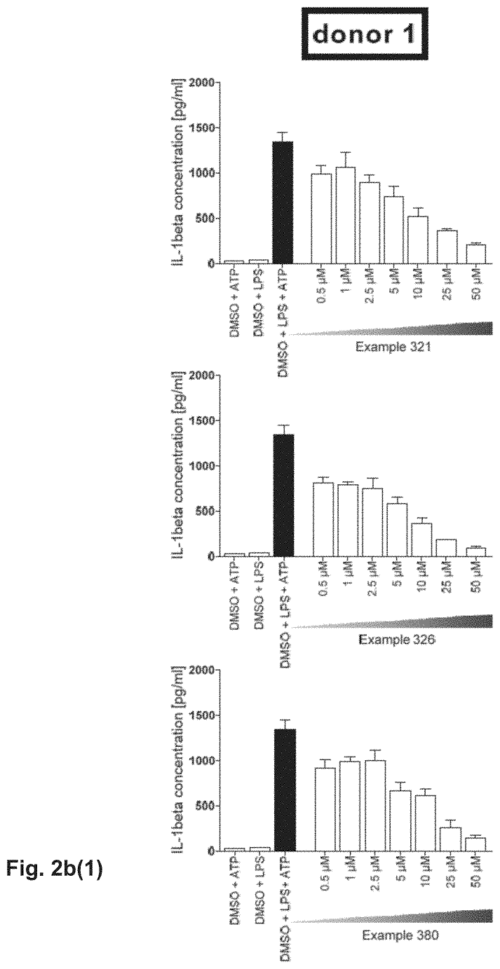

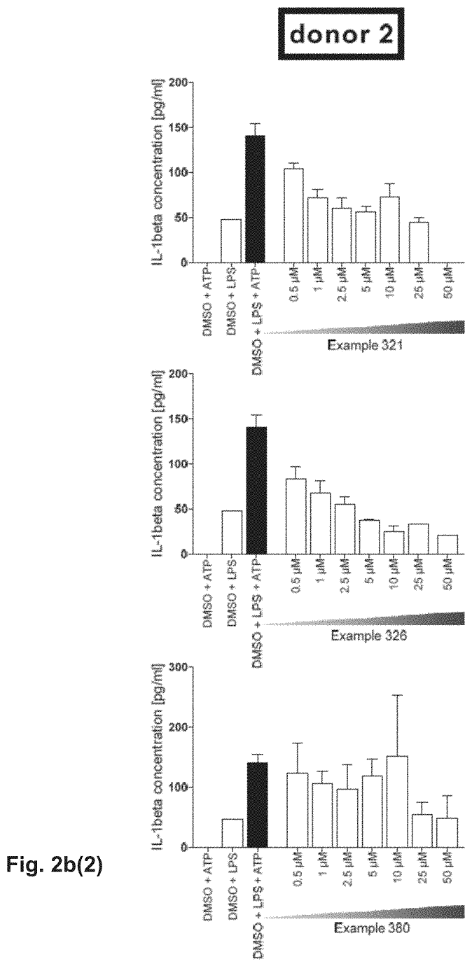

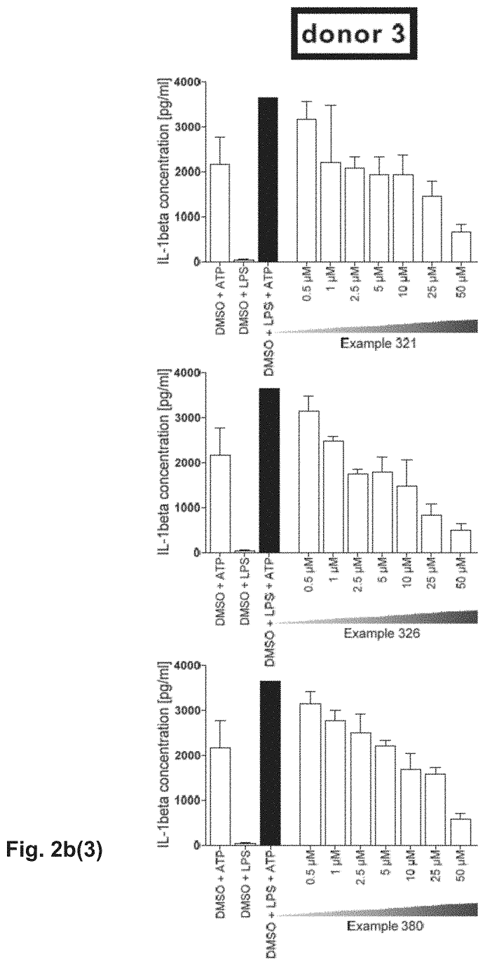

In this assay, ex vivo, the blood of healthy female volunteers is first sensitized with lipopolysacharide (LPS) and then stimulated with ATP to trigger the release of Interleukin 1beta (IL-1β). In this system, the efficacy of P2X4 antagonists on the production of IL-1β in whole blood was tested. The cells were first treated with 100 ng/ml LPS for 2 h and then stimulated with 3 mM ATP and treated in triplicates with examples 19, 28, 39, 321, 326 and 380 at different concentrations. After 1 h incubation, supernatant was taken and following centrifugation IL-1β in the supernatant was assayed using standard ELISA kits. The assay was performed with blood from three different donors (see FIGS. 2a and 2b).

FIGS. 2a and 2b as nonbinding explanatory example of compounds according to the invention represents the effect of the compounds according to examples 19, 28, 39, 321, 326 and 380 on the generation of IL-1β inhuman whole blood after ATP stimulation following priming of the cells with lipopolysacaride for two hours and indicated treatment. Data show the absolute amount of IL-1β in μg/ml in the supernatant of blood from three donors on the y-axis and controltreatments and treatments with different concentrations of examples are indicated on the x-axis. For each bar the average of three technical replicates and SD are shown. The data show inhibition of IL-1β release by several but not all of the tested examples.

In Vivo Studies

tMCAO-Induced Ischemic Stroke Model in Rats—Compound Example 39

Transient middle cerebral artery occlusion (tMCAO) was performed in approximately 3 months old male Sprague Dawley (SD) rats according to the method described by Schmid-Elsaesser et al. [Stroke. 1998; 29(10):2162-2170]. In particular, the right common carotid artery (CCA) was exposed through a midline neck incision and carefully dissected free from surrounding nerves and fascia—from its bifurcation to the base of the skull. The occipital and superior thyroid artery branches of the external carotid artery (ECA) were isolated and these branches were coagulated. The ECA was dissected further distally and coagulated along with the terminal lingual and maxillary artery branches, just before their bifurcation. The internal carotid artery (ICA) was isolated and carefully separated from the adjacent vagus nerve, and the pterygopalatine artery was ligated close to its origin with a 5-0 nylon suture. Thereafter, a 4-0 silk suture was tied loosely around the mobilized ECA stump, and a 4 cm length of Doccol 4-0 monofilament suture (coated with silicone) was inserted through the proximal ECA into the ICA and thence into the circle of Willis, effectively occluded the MCA. The surgical wound was closed and the animals were returned to their cages for recovery from anesthesia. Two hours after occlusion, rats were re-anesthetized and the monofilament was withdrawn to allow reperfusion. The wound was closed again and rats were returned to their cages.

Four groups of rats with 12-15 rats per group were included into the study. Two groups were subjected to tMCAO and 2 groups to sham operated animals (group 1=sham without treatment, group 2=sham with vehicle treatment, group 3=tMCAO with vehicle treatment, group 4=tMCAO with P2X4-antagonist treatment). Groups 2, 3, and 4 were treated for seven days twice a day by per os administration of the vehicle or P2X4-antagonist starting one hour before surgery. The modified neurologic severity score (mNSS) was used to grade and evaluate neurological functions [Li et al., Neurology 2001, 56: 1666-1672]. The mNSS is a composite of motor, sensory, reflex and balance tests and was graded on a scale of 0 to18 (normal score is 0 and maximum deficit score is represented by 18). All rats were subjected to the mNSS test before surgery for including only animals with normal mNSS. Two hours post-tMCAO, only rats with a mNSS equal or more than 10 were included into the study. The mNSS test was also performed on days 1, 2, 8, 15, 22 and 29 after surgery.

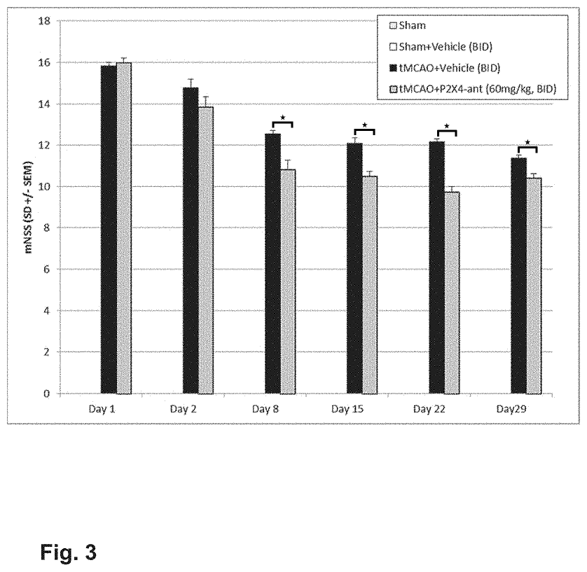

From day 8 on the P2X4-antagonist treated tMCAO-group led to a significant smaller mNSS than the vehicle treated tMCAO-group (p<5%; two-way ANOVA statistical analysis followed by Bonferroni post-hoc comparisons). Both sham-groups showed a mNSS of 0 at each time point (see FIG. 3).

tMCAO-Induced Ischemic Stroke Model in Mice

Transient middle cerebral artery occlusion (tMCAO) was performed in 8-10 weeks old male C57BL/6N mice according to the method described by Hata et al. [J Cereb Blood Flow Metab. 2000; 20(6):937-946]. In particular, the left common carotid artery (CCA) was exposed through a midline neck incision and carefully dissected free from surrounding nerves and fascia and ligated in anaesthetized mice. Then, the left external carotid artery (ECA) on the same side was separated and also ligated. After obtaining good view of the dissected internal carotid artery (ICA) and the pterygopalatine artery (PA), both arteries were clipped. Thereafter, a 8-0 nylon monofilament (Ethilon; Ethicon, Norderstedt, Germany) coated with silicon resin (Xantopren; Bayer Dental, Osaka, Japan) was introduced through a small incision into the common carotid artery and advanced 9 mm distal to the carotid bifurcation for occlusion of the MCA. The tip diameter of the thread (0.15 to 0.20 mm) was selected to match the body weight of the animals. The surgical wound was closed and the animals were returned to their cages for recovery from anesthesia. Fourty-five minutes after occlusion, the mice were re-anesthetized and the monofilament was withdrawn to allow reperfusion. The wound was closed and mice were returned to their cages.

Four groups of mice with 10-15 mice per group were included into the study. Three groups were subjected to tMCAO and 1 group to sham operated animals (group 1=sham without treatment, group 2=tMCAO with reference compound MK-801, group 3=tMCAO with vehicle treatment, group 4=tMCAO with P2X4-antagonist treatment). Group 2 was treated with the reference compound MK-801 only once per animal intraperitoneally 15 minutes before stroke surgery in a dose of 3 mg/kg body weight in a dose volume of 5 ml/kg body weight. Groups 3 and 4 were treated for 14 days twice a day by per os administration of the vehicle or P2X4-antagonist (60 mg/kg body weight) starting one hour before surgery, both in a dose volume of 5 ml/kg body weight.

Four different senso-motoric tests [modified Neurologic Severity Score (mNSS), Adhesive Removal Test (ART), Corner Test (CoT), and Cylinder Test (CT)] were included as read out-parameters for measuring drug treatment effects.

The modified Neurologic Severity Score (mNSS) was performed before surgery and on days 1, 7, 14, 21 and 28 after tMCAO or sham surgery. The mNSS used in this study was modified according the neuroscores published in Orsini et al. [Circulation. 2012; 126(12):1484-1494] and De Simoni et al. [J Cereb Blood Flow Metab. 2003; 23(2):232-239]. The mNSS was used to evaluate the general status and focal neurologic dysfunction after tMCAO. The score ranges from 0 (no deficits) to 39 (representing the poorest performance in all items) and is calculated as the sum of the general and focal deficits. The mNSS results were expressed as a composite neurological score, which included the following general deficits (scores): hair (0 to 2), ears (0 to 2), eyes (0 to 3), posture (0 to 3), spontaneous activity (0 to 3), and the following focal deficits (scores): body symmetry (0 to 2), gait (0 to 4), climbing on a surface inclined at 45 (0 to 3), circling behavior (0 to 3), forelimb symmetry (0 to 4), circling behavior (0 to 3), whisker response to light touch (0 to 4), and gripping test of the forepaws (0 to 3).

All mice were subjected to the mNSS test before surgery for including only animals with normal mNSS. Twenty four hours after tMCAO, only mice with a mNSS equal or more than 8 were included into the study.

The adhesive removal test (ART) was used to measure somatosensory deficits. A piece of adhesive-backed paper dot (approximately 2 mm 0) was used as a tactile stimulus by fixing them on the plantar region of the right forelimb. One week prior to tMCAO surgery, each animal received 3 ART trials per day at two days. If the animals failed to remove the stimulus on the second day of conditioning in a mean time of 60 seconds, an additional conditioning day was added. If the animal failed to remove the stimulus even after the extra added trial day within 60 seconds, then the animal was excluded from the study. The ART was performed before surgery, and on days 7, 14, 21 and 28 after surgery. At each test day, the test was performed 3 times per animal. The time in the three trials required to detect and to remove the adhesive stimuli from the right forelimb was recorded and evaluated.

The corner test (CoT) was used to measure stroke related forelimb akinesia. The corner test system was produced by use of four boards which have been glued together to form two opposite corners with angles of 30° whereby one of these corners contain a small gap to encourage the mice to find this respective corner. This corner test system was placed in a cage with standard bedding. For performing the CoT, the camera was started, the mouse placed in the middle of the rectangle and recorded for 10 min. The mouse tried to reach the corner with the gap. However, mice with a stroke cannot walk straight ahead. Therefore they turned rather to the left or to the right side and the number of turns to the left and right were counted from the records for the evaluation. The CoT was performed before surgery, and on days 14, and 28 after surgery.

The cylinder test (CT) was used to investigate the exploratory behavior by counting the spontaneous forelimb use. To perform this test, mouse was put in a transparent cylinder (12 cm diameter and 20 cm height) for 5 min. A mirror was placed behind the cylinder at an angle to permit recording of forelimb movements whenever the animal will turn away from the observer. The cylinder was high enough to prevent the animal of reaching the top edge by rearing. No habituation to the cylinder prior to observation was allowed. The number of wall contacts performed independently with the left and the right forepaw and the parallel contact with both forepaws was counted per mouse per session. Only supporting contacts were counted, i.e. full appositions of the paws with open digits to the cylinder walls. The CT was performed on days 14, and 28 after surgery.

1: A method for treatment or prophylaxis of a brain ischemia, ischemic brain injury, Ischemic Stroke (IS), haemorrhagic stroke, traumatic brain injury, or spinal cord injury, comprising administering to a patient in need thereof a pharmaceutically effective amount of a compound of formula (I), or an N-oxide, a salt, a hydrate, a solvate, a tautomer or a stereoisomer of said compound, or a salt of said N-oxide, tautomer or stereoisomer; 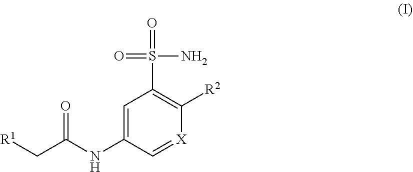
wherein
X is C or N
R¹ is 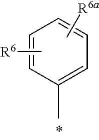
wherein * indicates the point of attachment of said group to the rest of the molecule and R⁶ and R^{6a} are independently from each other a fluorine, a chlorine, a methoxy or a hydrogen;
R² is 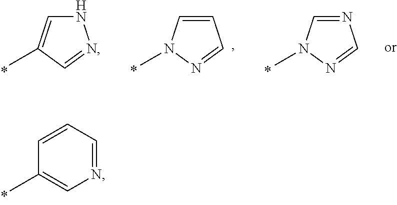
wherein * indicates the point of attachment of said group to the rest of the molecule and said group is optionally independently substituted one to two times with R¹¹;
each R¹¹ is, independently halogen, cyano, C₁-C₄-alkyl, C₁-C₄-haloalkyl, C₁-C₄-hydroxyalkyl, C₁-C₄-alkoxy, C₁-C₄-haloalkoxy, (C₁-C₃-alkoxy)-ethyl-, methoxy-ethyl-, or C₃-C₆-cycloalkyl.

2-3. (canceled)

4: The method according to claim 1, wherein the compound is
2-(2-Chlorophenyl)-N-{3-sulfamoyl-4-[4-(trifluoromethyl)-1H-pyrazol-1-yl]phenyl}acetamide;
2-(2-Fluorophenyl)-N-{3-sulfamoyl-4-[4-(trifluoromethyl)-1H-pyrazol-1-yl]phenyl}-acetamide;
2-(2-Chlorophenyl)-N-[4-(3-chloro-1H-1,2,4-triazol-1-yl)-3-sulfamoylphenyl]¬acetamide;
2-(2-Chlorophenyl)-N-[4-(4-chloro-1H-pyrazol-1-yl)-3-sulfamoylphenyl]acetamide;
2-(2-Chlorophenyl)-N-[4-(4-fluoro-1H-pyrazol-1-yl)-3-sulfamoylphenyl]acetamide;
N-[4-(4-Bromo-1H-pyrazol-1-yl)-3-sulfamoylphenyl]-2-(2-chlorophenyl)acetamide;
2-(2-Chlorophenyl)-N-[4-(4-cyano-1H-pyrazol-1-yl)-3-sulfamoylphenyl]acetamide;
N-[4-(4-Chloro-1H-pyrazol-1-yl)-3-sulfamoylphenyl]-2-(4-methoxyphenyl)acetamide;
N-{6-[1-(Difluoromethyl)-1H-pyrazol-4-yl]-5-sulfamoylpyridin-3-yl}-2-(2-fluorophenyl)acetamide;
2-(2-Chlorophenyl)-N-{4-[1-(difluoromethyl)-1H-pyrazol-4-yl]-3-sulfamoylphenyl}acetamide;
2-(2-Chlorophenyl)-N-{3-sulfamoyl-4-[5-(trifluoromethyl)pyridin-3-yl]phenyl}¬acetamide;
2-(2-Chlorophenyl)-N-[4-(1-cyclopropyl-1H-pyrazol-4-yl)-3-sulfamoylphenyl]acetamide;
2-(2-Chlorophenyl)-N-{5-sulfamoyl-6-[4-(trifluoromethyl)-1H-pyrazol-1-yl]pyridin-3-yl}acetamide;
2-(2-Fluorophenyl)-N-{5-sulfamoyl-6-[4-(trifluoromethyl)-1H-pyrazol-1-yl]pyridin-3-yl}acetamide; or
2-(2-Chlorophenyl)-N-[6-(4-chloro-1H-pyrazol-1-yl)-5-sulfamoylpyridin-3-yl]acetamide;
or an N-oxide, a salt, a hydrate, a solvate, a tautomer or a stereoisomer of said compound, or a salt of said N-oxide, tautomer or stereoisomer.

5: The method according to claim 1, wherein the compound is 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a pharmaceutically acceptable salt thereof, or a mixture of same.

6: The method according to claim 1, comprising administering the compound, or the N-oxide, the salt, the hydrate, the solvate, the tautomer or the stereoisomer of said compound, or the salt of said N-oxide, tautomer or stereoisomer, from the onset of the disease up to about one month, or up to about three weeks, or up to about two weeks, or up to about ten days.

7: The method according to claim 1, comprising administering the compound, or the N-oxide, the salt, the hydrate, the solvate, the tautomer or the stereoisomer of said compound, or the salt of said N-oxide, tautomer or stereoisomer, in combination or as co-medication with a antithrombotic agents

8: The method according to claim 1, comprising administering the compound, or the N-oxide, the salt, the hydrate, the solvate, the tautomer or the stereoisomer of said compound, or the salt of said N-oxide, tautomer or stereoisomer, in combination or as co-medication with Heparin, or Low-molecular-weight heparins or Danaparoid; or Argatroban, or Antithrombin or Protein C; or Aspirin, or Clopidogrel, or Abciximab, or Eptifibatide (Integrilin).

9: A parenteral formulation of a compound of formula I, or a stereoisomer, a tautomer, an N oxide, a hydrate, a solvate, or a salt thereof, particularly a pharmaceutically acceptable salt thereof, or a mixture of same 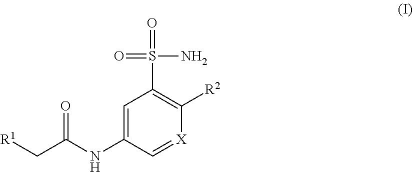
wherein
X is C or N
R¹ is 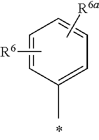
wherein * indicates the point of attachment of said group to the rest of the molecule and R⁶ and R^{6a} are independently from each other a fluorine, a chlorine, a methoxy or a hydrogen:
R² is 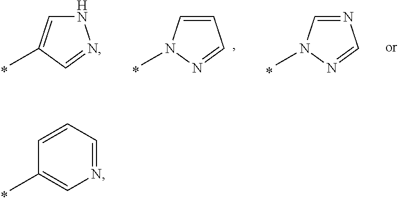
wherein * indicates the point of attachment of said group to the rest of the molecule and said group is optionally independently substituted one to two times with R₁₁;
each R¹¹ is, independently halogen, cyano, C₁-C₄-alkyl, C₁-C₄-haloalkyl, C₁-C₄-hydroxyalkyl, C₁-C₄-alkoxy, C₁-C₄-haloalkoxy, (C₁-C₃-alkoxy)-ethyl-, methoxy-ethyl-, or C₃-C₆-cycloalkyl.

10: A parenteral formulation of 2-(2-Chlorophenyl)-N-[4-(4-cyano-1H-pyrazol-1-yl)-3-sulfamoylphenyl]acetamide or a stereoisomer, a tautomer, an N oxide, a hydrate, a solvate, or a salt thereof, particularly a pharmaceutically acceptable salt thereof.

11: The parenteral formulation according to claim 6 wherein the parenteral formulation is a parenteral formulation for intravenous administration.

12: The parenteral formulation according to claim 7, wherein the parenteral formulation is a parenteral formulation for intravenous administration.
